(19) United States
(12) Patent Application Publication
(10) Pub. No.: US 2024/0392275 A1
(43) Pub. Date: Nov. 28, 2024

(54) LYSOSTAPHIN VARIANTS AND METHODS OF USE THEREOF

(71) Applicant: BiomEdit, LLC, Fishers, IN (US)

(72) Inventor: Yong-Fu LI, Fishers, IN (US)

(21) Appl. No.: 18/694,162

(22) PCT Filed: Sep. 22, 2022
(86) PCT No.: PCT/US2022/044348

§ 371 Date: Mar. 21, 2024

Related U.S. Application Data
(60) Provisional application No. 63/246,964, filed on Sep. 22, 2021.

(51) Int. Cl. C12N 9/52; A61K 38/48; A61K 45/06

(52) U.S. Cl. CPC C12N 9/52; A61K 38/4886; A61K 45/06; C12Y 304/24075

(57) ABSTRACT

The present disclosure provides lysostaphin variants comprising one or more amino acid mutations in the lysostaphin sequence as well as formulations and methods of using the lysostaphin variants which exhibit desirable properties and provide benefits for the treatment of infections in animals.

### CROSS-REFERENCE TO RELATED APPLICATIONS

This application claims priority from U.S. Provisional Patent Application Ser. No. 63/246,964, filed Sep. 22, 2021, which is herein incorporated by reference in its entirety.

### INCORPORATION OF SEQUENCE LISTING

The sequence listing in ST.26 XML format entitled 2948-23_PCT.xml, created on Sep. 21, 2022, comprising 159,421 bytes, prepared according to 37 CFR 1.822 to 1.824, submitted concurrently with the filing of this application, is incorporated herein by reference in its entirety.

### TECHNICAL FIELD

The invention relates to lysostaphin variants comprising a lysostaphin sequence and one or more amino acid mutations. The invention includes compositions, methods, and formulations utilizing the lysostaphin variants for the treatment of infections in animals.

### BACKGROUND

Lysostaphin (UniProt entry P10547) is an antimicrobial agent belonging to a major class of antimicrobial peptides and proteins known as bacteriocins. Bacteriocins are bacterial antimicrobial peptides which generally exhibit bactericidal activity against other bacteria. In particular, lysostaphin is known as a metalloendopeptidase of Staphylococcus simulans and can function as a bacteriocin against various bacteria and is classified as a Class III bacteriocin.

Lysostaphin is encoded by the lss gene and has been cloned and expressed heterologously in Escherichia coli, in a simian kidney cell line, and in Lactococcus lactis. Lysostaphin is a monomeric zinc-containing metallo-enzyme of 246 amino acids, a molecular mass of −27 kDa, a pI of 9.5 and a pH optimum of 7.5.

The lysostaphin protein is encoded by the lss gene of Staphylococcus simulans (GenbankID: L66883.1) and encodes a preproenzyme of 493 amino acids (SEQ ID NO:2) (GenbankID: AAB53783.1), an N-terminal 36 amino acid leader peptide initiates secretion. into the secretory pathway by. Prolysostaphin is 4.5-fold less active than mature lysostaphin and the N-terminal repeats are removed to yield the fully activated lysostaphin molecule. The lysostaphin molecule consists of two distinct domains: (i) an N-terminal peptidase domain responsible for the catalytic activity of the protein (referred to as CatD, SEQ ID NO:4, 142 amino acids) and (ii) a C-terminal targeting domain (CWT; SEQ ID NO:8, 102 amino acids) involved in binding to the peptidoglycan substrate. The CWT domain is a member of the Src Homology 3 domain superfamily; c117036 (CDD:450141) and binds to the peptide cross-bridge (the penta-Glycine) in the gram-positive bacterial cell wall.

Lysostaphin has many attractive features for use as an antimicrobial agent: (i) it has activity against non-dividing as well as dividing cells, (ii) it is digested by intestinal proteinases, having no influence on the gut microbiota, (iii) it has no toxicity, (iv) it is relatively stable when conjugated with polyethylene glycol (PEG), and (v) it maintains its activity in human serum. See Freire Bastos et al., Lysostaphin: A Staphylococcal Bacteriolysin with Potential Clinical Applications,” Pharmaceuticals 3:1139-1161 (2010) and references cited therein. Lysostaphin retains its bacteriolytic activity in vivo, without any undesirable immune reaction, despite the presence of high-neutralizing antibody titer. See Dajcs et al., “Immunity to lysostaphin and its therapeutic value for ocular MRSA infections in the rabbit,” Invest. Opthalmol. Visual Sci. 43:3712-3716 (2002).

Lysostaphin has shown promise in a number of therapeutic, veterinary, and medical applications. Lysostaphin is highly effective against S. aureus strains independent of the potential drug resistance with a minimum inhibitory concentration of ranges between 0.001 and 0.064 μg/ml. Lysostaphin is active against both dividing and non-dividing cells including S. aureus embedded in biofilms. See Jayakumar et al., “Therapeutic applications of lysostaphin,” J. Applied Microbiology 131:1072-1082 (2020). Lysostaphin has been shown to be effective in nasal decolonization, wound infections (skin), burn wounds, eye infections, bone infections, endocarditis and sepsis. See id. at Table 1. In veterinary medicine, lysostaphin has shown effectiveness in treating mastitis. In addition, lysostaphin is effective against S. aureus biofilms but also disrupted the extracellular matrix of S. aureus biofilms in vitro on plastic and glass surfaces at concentrations as low as 1 μg/ml. We et al., Antimicrobial Agents and Chemotherapy, November 2003, p. 3407-3414. Cathetors coated with lysostaphin maintain activity after four days. Shah et al. “Lysostaphin-coated catheters eradicate Staphylococccus aureus challenge and block surface colonization,” Antimicrob Agents Chemother. 48(7):2704-7 (2004).

However, lysostaphin can be problematic as a therapeutic agent and other purposes due to issues with its stability. Class III bacteriocins are known to be heat labile. For instance, degradation of lysostaphin can occur during fermentation. In particular, self degradation of lysostaphin has been observed at room temperature and at higher temperatures, for instance about 37° C. Accordingly, new compositions and formulations for variants of lysostaphin are highly desirable. Ideally, such lysostaphin variants would provide a reduction in degradation that is observed with wild type lysostaphin and would be more stable as therapeutic agents.

### SUMMARY

The present specification comprises, in one form thereof, a lysostaphin variant comprising a lysostaphin sequence of SEQ ID NO:5 and at least one amino acid mutation selected from the group consisting of G25P, G25S, G25T, N27E, N27T, N27D, N27V, N27S, N27A, G29S, G29T, G56S, G62S, T126N, G143S, and S233A.

In another aspect, a lysostaphin variant comprising a lysostaphin sequence of SEQ ID NO:4 and at least one amino acid mutation selected from the group consisting of G25P, G25S, G25T, N27E, N27T, N27D, N27V, N27S, N27A, G29S, G29T, G56S, G62S, and T126N, is provided.

In aspects, the lysostaphin variants can be prepared as a composition comprising the lysostaphin variants of SEQ ID NO:4 and SEQ ID NO:5, either alone or combined with a second active agent.

Another aspect of the present specification provides for pharmaceutical compositions comprising a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A.

In an aspect of the present specification a method of treating an infection in an animal, the method comprising the steps of administering a lysostaphin variant, a composition of any lysostaphin variant, wherein the composition or the veterinary composition reduces one or more symptoms associated with the infection in the animal.

In another aspect, the specification provides for methods of treating an animal comprising the steps of administering the lysostaphin variant, a composition of a lysostaphin variant to an animal, wherein the composition or reduces the levels of Gram-positive bacterium in or on said animal.

In other aspects, the specification provides for recombinant nucleic acid vectors for the expression of lysostaphin variants comprising a transcription promoter, a nucleic acid sequence encoding a lysostaphin polypeptide sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A, and a transcriptional terminator.

In another aspect, the specification provides for methods of producing a lysostaphin variants comprising a lysostaphin sequence of SEQ ID NO:5 comprising at least one amino acid mutation selected from the group consisting of G25P, G25S, G25T, N27E, N27T, N27D, N27V, N27S, N27A, G29S, G29T, G56S, G62S, T126N, G143S, and S233A, said method comprising inducing expression of the lysostaphin polypeptide from the recombinant nucleic acid vector of a lysostaphin variant in a cell.

In an aspect, the present specification provides methods of treating microbial colonization of medical devices comprising applying a lysostaphin polypeptide variant to the surface of a medical device.

### BRIEF DESCRIPTION OF THE DRAWINGS

The present specification is disclosed with reference to the accompanying drawings, wherein:

FIG. 1 presents an exemplary SDS-PAGE analysis lysostaphin showing two bands, one at 27 kiloDalaton (kD) and a second at 17 kD, following fermentation of 28, 31, 47, 50, and 53 hours in an aspect of the present specification.

FIG. 2 presents shows intact mass analysis of the 17 kDa band.

FIG. 3 presents an exemplary SDS-PAGE analysis of a sample of lysostaphin expressed in P. lentis, purified, and in solution in 50 mM Tris-HCl, 0.15 M NaCl pH 7.5.

FIG. 4 presents an SDS-PAGE analysis of the purified samples of FIG. 3, aliquoted stored for 14 days at −80° C., 4° C., RT, and 37° C. At day 14, degradation of the purified protein is observed compared to the samples at day 0.

FIG. 5 presents results from densitometry performed on the daily SDS-PAGE analysis of stored lysostaphin samples stored at −80° C., 4° C., RT, and 37° C. and lytic activity of lysostaphin against S. pseudintermedius. FIG. 5A plots the resulting densities versus time.

FIG. 5B presents the lysostaphin activity against S. pseudintermedius as measured by a turbidity reduction assay of lysostaphin at 50 Bug/ml.

FIG. 6 presents SEC-HPLC of incubated lysostaphin samples showing four bands of lower molecular weights from the SDS-PAGE lysostaphin analysis.

FIG. 7 presents an SDS-Page analysis showing four bands selected for mass spec analysis.

FIG. 8 presents a graphical analysis of identity of bands 1 to 4 (degradation products 1 to 4) of FIG. 7 relative to SEQ ID NO:2 as determined by mass spec analysis.

FIG. 9 presents a summary of the mass spec analysis of the analysis shown in FIG. 8. Major cleavage points are shown with “I”, missing sequence ASGFYVSGT (SEQ ID NO:6) is shown underlined, a deamidation site at N27 is shown in bold, and a cleavage site at N157-K158 identified in fermenters is shown in bold.

FIG. 10 graphically presents the lysostaphin structures showing various features including the Zn²⁺ binding site, the catalytic site (CAT), the CWD, a deamidation site at positions G25 to G29 (SEQ ID NO:32), a cleavage site between amino acids 55 and 56, between amino acids G61 and G63, between amino acids A143 and G144, and between amino acids N157 to K158.

FIG. 11 shows a turbidity reduction assay of lysostaphin at 50 Bug/ml in S. pseudintermedius.

FIG. 12 shows a turbidity assay in S. suis for various lysostaphin variants in 50 mM Tris-HCl pH8.0. The lytic enzyme PlySs2 (from S. suis) is shown as a positive control in both N-terminal histidine tagged (i.e., PlySs2NH) and C-terminal histidine tagged (PlySs2CH) forms.

FIG. 13 presents a graph of an exemplary turbidity assay in S. pseudintermedius (50 mM Tris, pH 8) for various lysostaphin variants. Four groups are presented based on the observed relative activities. Native lysostaphin (natLss), N-terminal histidine tagged lysostaphin (LssNH), N-terminal histidine tagged S. suis PlySs2 (PlySs2NH) and C-terminal histidine tagged S. suis PlySs2 (PlySs2CH) are included as positive controls.

FIG. 14 presents a graph of an exemplary turbidity assay in S. pseudintermedius (PBS) for various lysostaphin variants. Four groups are presented based on the observed relative activities. Native lysostaphin (natLss), N-terminal histidine tagged lysostaphin (LssNH), N-terminal histidine tagged S. suis PlySs2 (PlySs2NH) and C-terminal histidine tagged S. suis PlySs2 (PlySs2CH) are included as positive controls.

FIG. 15 presents a graph of an exemplary turbidity assay in S. aureus (50 mM, Tris, pH 8.0) for various lysostaphin variants. Native lysostaphin (natLss), N-terminal histidine tagged lysostaphin (LssNH), N-terminal histidine tagged S. suis PlySs2 (PlySs2NH) and C-terminal histidine tagged S. suis PlySs2 (PlySs2CH) are included as positive controls.

FIG. 16 presents a graph of an exemplary turbidity assay in S. aureus (in PBS) for various lysostaphin variants. Native lysostaphin (natLss), N-terminal histidine tagged lysostaphin (LssNH), N-terminal histidine tagged S. suis PlySs2 (PlySs2NH) and C-terminal histidine tagged S. suis PlySs2 (PlySs2CH) are included as positive controls.

FIG. 17 presents a graph of an exemplary turbidity assay in S. pseudintermedius (in 50 mM, Tris, pH 8.0) for various lysostaphin variants. Native lysostaphin (natLss), N-terminal histidine tagged lysostaphin (LssNH), N-terminal histidine tagged S. suis PlySs2 (PlySs2NH) and C-terminal histidine tagged S. suis PlySs2 (PlySs2CH) are included as positive controls.

FIG. 18 presents a graph of an exemplary turbidity assay in S. pseudintermedius (in PBS) for various lysostaphin variants. Native lysostaphin (natLss), N-terminal histidine tagged lysostaphin (LssNH), N-terminal histidine tagged S. suis PlySs2 (PlySs2NH) and C-terminal histidine tagged S. suis PlySs2 (PlySs2CH) are included as positive controls.

FIG. 19 presents results of an SDS-PAGE analysis of exemplary stability assays of various lysostaphin variants. Aliquots of 18 samples, diluted to 1 mg/ml with PBS, are stored at −80° C., 4° C., RT (22-25° C.), 37° C. and analyzed by SDS-PAGE.

FIG. 20 presents graphical results of an exemplary turbidity assay of samples stored at 37° C. in PBS for 17 days tested against in S. aureus in 50 mM, Tris, pH 8.0.

FIG. 21 presents graphical results of an exemplary turbidity assay of samples stored at 37° C. in PBS for 17 days tested against in S. aureus in PBS.

FIG. 22 presents graphical results of an exemplary turbidity assay of samples stored at 37° C. in PBS for 17 days tested against in S. pseudintermedius in 50 mM, Tris, pH 8.0.

FIG. 23 presents graphical results of an exemplary turbidity assay of samples stored at 37° C. in PBS for 17 days tested against in S. pseudintermedius in PBS.

FIG. 24 presents graphical results of an exemplary turbidity assay of samples stored at 4° C. in PBS for 17 days tested against in S. aureus in 50 mM, Tris, pH 8.0.

FIG. 25 presents graphical results of an exemplary turbidity assay of samples stored at 4° C. in PBS for 17 days tested against in S. aureus in PBS.

FIG. 26 presents graphical results of an exemplary turbidity assay of samples stored at 4° C. in PBS for 17 days tested against in S. pseudintermedius in 50 mM, Tris, pH 8.0.

FIG. 27 presents graphical results of an exemplary turbidity assay of samples stored at 4° C. in PBS for 17 days tested against in S. pseudintermedius in PBS.

FIG. 28 presents results of an exemplary stability assay of various lysostaphin variants stored at −80° C., 4° C., RT (22-25° C.), 37° C. and analyzed by SDS-PAGE after 32 days of storage.

FIG. 29 presents an exemplary SDS-PAGE analysis performed on samples stored at room temperature after 32 days. In lane 9, N-terminal histidine tagged lysostaphin (LssNH) is shown. In lane 10, Lss-NHal2 is shown. In lane 11, Lss-NHal3 is shown. In lane 12, Lss-NHCatD is shown. As shown in lane 11, degradation of Lss-NHal3 is not observed.

FIG. 30 presents graphical results of an exemplary turbidity assay of samples stored at 37° C. in PBS for 17 days tested against in S. pseudintermedius in PBS.

FIG. 31 presents graphical results of an exemplary turbidity assay of samples stored at 37° C. in PBS for 17 days tested against in S. aureus in 50 mM, Tris, pH 8.0.

FIG. 32 presents graphical results of an exemplary turbidity assay of samples stored at 4° C. in PBS for 17 days tested against in S. aureus in PBS.

FIG. 33 presents graphical results of an exemplary turbidity assay of samples stored at 4° C. in PBS for 17 days tested against in S. aureus in 50 mM, Tris, pH 8.0.

FIG. 34 presents graphical results of an exemplary turbidity assay of samples stored at 37° C. in PBS for 17 days tested against in S. pseudintermedius in PBS.

FIG. 35 presents graphical results of an exemplary turbidity assay of samples stored at 37° C. in PBS for 17 days tested against in S. pseudintermedius in 50 mM, Tris, pH 8.0.

FIG. 36 presents graphical results of an exemplary turbidity assay of samples stored at 4° C. in PBS for 17 days tested against in S. pseudintermedius in PBS.

FIG. 37 presents graphical results of an exemplary turbidity assay of samples stored at 4° C. in PBS for 17 days tested against in S. pseudintermedius in 50 mM, Tris, pH 8.0.

FIG. 38 presents graphical results of an exemplary turbidity assay of samples stored at 4° C. in PBS for 17 days tested against fresh S. aureus cells diluted 1:5 with PBS to 50 μg/ml (based on A280.

FIG. 39 presents graphical results of an exemplary turbidity assay of samples stored at 4° C. in PBS for 17 days tested against fresh S. pseudintermedius cells diluted 1:5 with PBS to 50 μg/ml (based on A280.

FIG. 40 is a graphical presentation of the relationship between SEQ ID NOs:1 to 8, wherein SEQ ID NOs:4 and 8 comprise lysostaphin variant sequences.

Corresponding reference characters indicate corresponding parts throughout the several views. The examples set out herein illustrates several aspects of the inventions but should not be construed as limiting the scope of the inventions in any manner.

### DETAILED DESCRIPTION

The present specification identifies lysostaphin cleavage sites and provides lysostaphin variants comprising one or more amino acid mutations in the lysostaphin sequence as well as formulations and methods of using the lysostaphin variants which exhibit desirable properties and provide associated benefits for the treatment of infections in animals. Various figures are included in the present specification and are identified in the Examples described herein. For turbidity reduction assays, optical density is shown on the y-axis and time on the x-axis.

In an aspect, a lysostaphin variant comprising a lysostaphin sequence and one or more amino acid mutations is provided. The present specification provides various lysostaphin sequences, including SEQ ID NO: 1, SEQ ID NO: 2, and SEQ ID NO: 3 as shown in Table 1:

TABLE 1
Lysostaphin isoforms from Staphylococcus simulans

| Sequence | |
|---|---|
| ID NO. | Sequence |
| SEQ ID | ATHEHSAQWLNNYKKGYGYGPYPLGINGGMHYGVDFFMNIGTPVKAISSG |
| NO: 1 | KIVEAGWSNYGGGNQIGLIENDGVHRQWYMHLSKYNVKVGDYVKAGQII |
| | GWSGSTGYSTAPHLHFQRMVNSFSNSTAQDPMPFLKSAGYGKAGGTVTPT |
| | PNTGWKTNKYGTLYKSESASFTPNTDIITRTTGPFRSMPQSGVLKAGQTIHY |
| | DEVMKQDGHVWVGYTGNSGQRIYLPVRTWNKSTNTLGVLWGTIK |
| SEQ ID | ASGFYVSGTAATHEHSAQWLNNYKKGYGYGPYPLGINGGMHYGVDFFMN |
| NO: 2 | IGTPVKAISSGKIVEAGWSNYGGGNQIGLIENDGVHRQWYMHLSKYNVKV |
| | GDYVKAGQIIGWSGSTGYSTAPHLHFQRMVNSFSNSTAQDPMPFLKSAGY |
| | GKAGGTVTPTPNTGWKTNKYGTLYKSESASFTPNTDIITRTTGPFRSMPQSG |
| | VLKAGQTIHYDEVMKQDGHVWVGYTGNSGQRIYLPVRTWNKSTNTLGVL |
| | WGTIK |
| SEQ ID | MKKTKNNYYTRPLAIGLSTFALASIVYGGIQNETHASEKSNMDVSKKVAEV |
| NO: 3 | ETSKAPVENTAEVETSKAPVENTAEVETSKAPVENTAEVETSKAPVENTAE |
| | VETSKAPVENTAEVETSKAPVENTAEVETSKAPVENTAEVETSKAPVENTA |
| | EVETSKAPVENTAEVETSKAPVENTAEVETSKAPVENTAEVETSKAPVENT |
| | AEVETSKAPVENTAEVETSKAPVENTAEVETSKALVQNRTALRAATHEHSA |
| | QWLNNYKKGYGYGPYPLGINGGMHYGVDFFMNIGTPVKAISSGKIVEAGW |
| | SNYGGGNQIGLIENDGVHRQWYMHLSKYNVKVGDYVKAGQIIGWSGSTG |
| | YSTAPHLHFQRMVNSFSNSTAQDPMPFLKSAGYGKAGGTVTPTPNTGWKT |
| | NKYGTLYKSESASFTPNTDIITRTTGPFRSMPQSGVLKAGQTIHYDEVMKQD |
| | GHVWVGYTGNSGQRIYLPVRTWNKSTNTLGVLWGTIK |

The one-letter codes for amino acids are as provided by IUPAC and WIPO sequence rules.

As described herein, lysostaphin variants comprise one or more amino acid mutations of a lysostaphin sequence. In an aspect, the lysostaphin variants comprise the amino acid sequence of SEQ ID NO:5 having at least one amino acid mutation selected from the group consisting of G25P, G25S, G25T, N27E, N27T, N27D), N27V, N27S, N27A, G29S, G29T, G56S, G62S, T126N, G143S, and S233A. Specific amino acid mutations are identified with amino acid numbering that correspond to the amino acid positions of SEQ ID NO: 1. Specific amino acid mutations of SEQ ID NO: 2, SEQ ID NO: 3, and SEQ ID NO:4 are also included in the present inventions, and are present at the position of SEQ ID NO: 2 and SEQ ID NO: 3 that correspond to the position of the amino acid mutation of SEQ ID NO: 1.

The one or more amino acid mutations of a lysostaphin sequence can be selected from the group consisting of the mutations in Table 2, and any combination thereof:

TABLE 2
Amino acid mutations of Lysostaphin variants

| | Amino Acid | Alternative Names |
|---|---|---|
| Amino Acid | Mutation(s) relative | Described Herein |
| Mutation No. | to SEQ ID NO: 1* | (e.g., see Figures) |
| 1 | G25P | NH1a |
| 2 | G25S | |
| 3 | G25T | NH1b |
| 4 | N27E | NH1c |
| 5 | N27T | NH1d |
| 6 | N27D | NH1f |
| 7 | N27V | NH1i |
| 8 | N27S | NH1g |
| 9 | N27A | NH1h |
| 10 | G29S | NH1e |
| 11 | G29T | |
| 12 | G56S | |
| 13 | G62S | |
| 14 | T126N | |
| 15 | G143S | NH2a |
| 16 | G144Q | NH2b |
| 17 | N157D | NH3b |
| 18 | K158Q | NH3a |
| 19 | S233A | |
| 20 | N27T/G29S/T126N/G143S/ | NHall |
| | N157D/S233A | |
| 21 | G25T/G29S/T126N/G143S/ | NHAl2 |
| | N157D/S233A | |
| 22 | G25T/N27S/G29S/T126N/ | NHAl3 |
| | G143S/N157D/S233A | |
| 23 | G25T/G29S/T126N/G143S/ | NHAl4 |
| | S233A | |
| 24 | G25T/G29S/G56S/T126N/ | NHAl5 |
| | G143S/S233A | |
| 25 | G25T/G28S/G56S/G62S/ | NHAl9 |
| | T126N/G143S/S233A | |

*Specific amino acid mutations of SEQ ID NO: 2, SEQ ID NO: 3, and 4 correspond to the position of the amino acid mutation of SEQ ID NO: 1 unless indicated otherwise.

The specification provides for, and includes, lysostaphin variants comprising a lysostaphin sequence of SEQ ID NO:5 wherein position 25 can be Glycine, Proline, Serine, or Threonine, position 27 can be N, E, F, D, V, S, or A, position 56 can be G or Serine, position 61 can be G or Serine, position 126 can be T or N, position 142 can be A or S, position 143 can be G or S, position 233 can be S or A, and wherein at least at least one amino acid mutation selected from the group consisting of G25P, G25S, G25T, N27E, N27T, N27D, N27V, N27S, N27A, G29S, G29T, G56S, G62S, T126N, G143S, and S233A. As provided herein, the lysostaphin variants exhibit a lower amount of degradation as compared to wild type lysostaphin. In aspects, compared to wild type lysostaphin, the lysostaphin variants retain higher activity after 17 days after incubation at 37° C. and 4° C. See FIGS. 20 to 27.

The present specification provides for, and includes, combinations of mutations in the lysostaphin variants. In aspects, the lysostaphin variants comprise one to eight amino acid changes. In an aspect, the lysostaphin variant comprises two amino acid changes. In another aspect, the lysostaphin variants comprise three amino acid changes. In other aspects, the lysostaphin variants comprise four amino acid changes. In another aspect, the lysostaphin variants comprise five amino acid changes. In a further aspect, the lysostaphin variants comprise six amino acid changes. In yet another aspect, the lysostaphin variants comprise seven amino acid changes. In an aspect, the lysostaphin variant comprises the amino acid mutations N27T, G29S, T126N, G143S, N157D, and S233A. In another aspect, the lysostaphin variant comprises the amino acid mutations G25T, G29S, G143S, and N157D.

Provided for, and included, are peptides comprising SEQ ID NO:5, further comprising a polypeptide having the amino acid sequence of SEQ ID NO:6 joined via a peptide bond between the carboxy terminus of SEQ ID NO:6 and the amino terminus of SEQ ID NO:5, wherein the combined peptides are lysostaphin variants corresponding to SEQ ID NO:2. In an alternative aspect, SEQ ID NO:5 further comprises a polypeptide having the amino acid sequence of SEQ ID NO:7 joined via a peptide bond between the carboxy terminus of SEQ ID NO:7 and the amino terminus of SEQ ID NO:5, wherein the combined peptide corresponds to lysostaphin variants of SEQ ID NO:3. The relationship between SEQ ID NOs:1 to 8 are illustrated in FIG. 40.

In aspects, the lysostaphin variant can a polypeptide having the amino acid sequence of any one of SEQ ID NOs:9 to 108.

Also provided for, and included, are additional mutations known in the art, including those of Blazanovic et al., “Structure-based redesign of lysostaphin yields potent antistaphylococcal enzymes that evade immune cell surveillance,” Molecular Therapy—Methods & Clinical Development 2:15021 (2015). These including the mutations of N12G, N13H, 141E, K46H, L83M, Y93H, K95E, I99Q, R118T, N121G, and S124G. Also included are mutations reported by Shen et al. that reduce glycosylation, N125Q, N232Q, S126P, T127A, and combinations thereof. See Shen et al., “Design and High Expression of Non-glycosylated Lysostaphins in Pichia pastoris and Their Pharmacodynamic Study,” Front. Microbiol. 12:637662 (2021).

In an aspect, the lysostaphin variant comprises the catalytic domain of lysostaphin (i.e., amino acids 1-142 of SEQ ID NO: 1). This particular variant is referred to herein as “CatD”, “Nhcat”, “Cat”, and/or “cat”. The amino acid sequence of the wild type CatD polypeptide variant is as follows:

(SEQ ID NO: 4)
ATHEHSAQWLNNYKKGYGYGPYPLGINGGMHYGVDFFMNIGT
PVKAISSGKIVEAGWSNYGGGNQIGLIENDGVHRQWYMHLSKY
NVKVGDYVKAGQIIGWSGSTGYSTAPHLHFQRMVNSFSNSTAQ
DPMPFLKSAGYGKA

The present specification provides for, and includes a lysostaphin variant comprising a lysostaphin sequence of SEQ ID NO:4 wherein position 25 can be Glycine, Proline, Serine, or Threonine, position 27 can be Asparagine (N), Glutamic Acid (E), Phenylalanine (F), Aspartic Acid (D), Valine (V), Serine (S), or Alanine (A), position 56 can be Glycine or Serine, position 61 can be Glycine or Serine, and position 126 can be Threonine (T) or Asparagine, and comprising at least one amino acid mutation selected from the group consisting of G25P, G25S, G25T, N27E, N27T, N27D, N27V, N27S, N27A, G29S, G29T, G56S, G62S, and T126N. As provided herein, the lysostaphin variants exhibit a lower amount of degradation as compared to wild type lysostaphin. In aspects, compared to wild type lysostaphin, the lysostaphin variants retain higher activity after 17 days after incubation at 37° C. and 4° C. See FIGS. 20 to 27.

In aspects, the lysostaphin variants of SEQ ID NO:4 comprises a polypeptide having the amino acid sequence of any one of SEQ ID NOs:9 to 22. In further aspects, the lysostaphin variants of SEQ ID NO:4 can comprise an addition serine at the carboxy terminal end (e.g., position 143). In aspects, the lysostaphin variants having a serine at position 143 comprise a polypeptide having the amino acid sequence of any one of SEQ ID NOs:28 to 33.

The present specification provides for, and includes, combinations of mutations in the lysostaphin variants of SEQ ID NO:4. In aspects, the lysostaphin variants comprise one to five amino acid changes. In an aspect, the lysostaphin variant comprises two amino acid changes. In another aspect, the lysostaphin variants comprise three amino acid changes. In other aspects, the lysostaphin variants comprise four amino acid changes. In an aspect, the lysostaphin variant comprises the amino acid mutations N27T, G29S, T126N, and G143S. In another aspect, the lysostaphin variant comprises the amino acid mutations G25T, G29S, and G143S.

Provided for, and included, are peptides comprising SEQ ID NO:4 variants, further comprising a polypeptide having the amino acid sequence of SEQ ID NO:6 joined via a peptide bond between the carboxy terminus of SEQ ID NO:6 and the amino terminus of SEQ ID NO:4, wherein the combined peptides are lysostaphin variants corresponding to SEQ ID NO:2. See FIG. 40. In aspects, the lysostaphin variants of the combined amino acid sequences of SEQ ID NO:4 and SEQ ID NO:6 comprise a polypeptide having the amino acid sequence of any one of SEQ ID NOs: 59 to 72.

In an alternative aspect, SEQ ID NO:4 variants further comprises a polypeptide having the amino acid sequence of SEQ ID NO:7 joined via a peptide bond between the carboxy terminus of SEQ ID NO:7 and the amino terminus of SEQ ID NO:4, wherein the combined peptide corresponds to lysostaphin variants of SEQ ID NO:3. See FIG. 40. In aspects lysostaphins variant comprising the SEQ ID NO:4 variants and amino terminal amino acids set forth in SEQ ID NO:7 are variant polypeptides having the amino acid sequence of any one of SEQ ID NOs:84 to 97.

Provided for, and included, are peptides comprising SEQ ID NO:4 variants, further comprising a polypeptide having the amino acid sequence of SEQ ID NO:8 joined via a peptide bond between the amino terminus of SEQ ID NO:8 and the carboxy terminus of SEQ ID NO:4 variants, wherein the combined peptides are lysostaphin variants corresponding to SEQ ID NO:1. See FIG. 40. In further aspects, the polypeptide having the amino acid sequence of SEQ ID NO:8 can comprise one or more amino acid mutations.

further comprising a polypeptide having the amino acid sequence of SEQ ID NO:8 comprising one or more amino acid mutations selected from the group consisting of G1S, G2Q, N15D, K16Q, and S91A (as numbered according to SEQ ID NO:8) joined via a peptide bond between the amino terminus of SEQ ID NO:8 and the carboxy terminus of SEQ ID NO:4. In aspects, the SEQ ID NO:8 variants can comprise polypeptides having the amino acid sequence of SEQ ID NOs: 23 to 27. Also included, are lysostaphin variants of wild type lysostaphin polypeptides. In aspects, the lysostaphin variants the amino acid variants of SEQ ID NO:8 comprise a polypeptide having the amino acid sequence of any one of SEQ ID NOs:73 to 83.

Also provided for, and included, are additional mutations known in the art and applied to the SEQ ID NO:4 variants, including the mutations of N12G, N13H, 141E, K46H, L83M, Y93H, K95E, I99Q, R118T, N121G, and S124G of Blazanovic et al. Also included are mutations reported by Shen et al. that reduce glycosylation, N125Q, N232Q, S126P, T127A, and combinations thereof. See Shen et al., “Design and High Expression of Non-glycosylated Lysostaphins in Pichia pastoris and Their Pharmacodynamic Study,” Front. Microbiol. 12:637662 (2021).

The lysostaphin variants of the present specification exhibit reduced degradation compared to wild type lysostaphin. In aspects, the degradation is self-degradation. In other aspects, the degradation occurs during fermentation. As provided herein, the degradation is reduced during fermentation of lysostaphin variants compared to wild type lysostaphin. In aspects, the degradation is reduced at room temperature compared to wild type lysostaphin. In other aspects, the degradation is reduced at 20° C. compared to wild type lysostaphin. In yet other aspects, wherein the degradation is reduced at 37° C. compared to wild type lysostaphin. In aspects, the comparison is performed after 7 days. In another aspect, the comparison is done after 14 days. In aspects, the stability of the lysostaphin variants are compared to wild type lysostaphin after 17 days. In yet further aspects, the degradation is compared at 32 days or more at 4° C., room temperature (22 to 25° C.), or 37° C. In aspects, degradation is measured as the activity of lysostaphin using a turbidity reduction assay. In other aspects, degradation can be measured directly, for example by SDS-PAGE analysis, chromatography, mass spec analysis, or combinations thereof. Method for comparing stability and degradation are provided below and known in the art.

The present specification provides for an included lysostaphin variants having the SEQ ID numbers as provided in Table 3.

TABLE 3
Lysostaphin mutations and variants

| | | Variants | Variants | Variants | Variants | |
|---|---|---|---|---|---|---|
| AA | | based on | based on | based on | based on | |
| Mutation | | SEQ ID | SEQ ID | SEQ ID | SEQ ID | |
| No. | Mutation(s) | NO: 1 | NO: 2 | NO: 3 | NO: 4 | Alt. ID |
| 1 | G25P | 34 | 59 | 84 | 9 | NH1a |
| 2 | G25S | 35 | 60 | 85 | 10 | |
| 3 | G25T | 36 | 61 | 86 | 11 | NH1b |
| 4 | N27E | 37 | 62 | 87 | 12 | NH1c |
| 5 | N27T | 38 | 63 | 88 | 13 | NH1d |
| 6 | N27D | 39 | 64 | 89 | 14 | NH1f |
| 7 | N27V | 40 | 65 | 90 | 15 | NH1i |
| 8 | N27S | 41 | 66 | 91 | 16 | NH1g |
| 9 | N27A | 42 | 67 | 92 | 17 | NH1h |
| 10 | G29S | 43 | 68 | 93 | 18 | NH1e |
| 11 | G29T | 44 | 69 | 94 | 19 | |
| 12 | G56S | 45 | 70 | 95 | 20 | |
| 13 | G62S | 46 | 71 | 96 | 21 | |
| 14 | T126N | 47 | 72 | 97 | 22 | |
| 15 | G143S | 48 | 73 | 98 | 23* | NH2a |
| 16 | G144Q | 49 | 74 | 99 | 24* | NH2b |
| 17 | N157D | 50 | 75 | 100 | 25* | NH3b |
| 18 | K158Q | 51 | 76 | 101 | 26* | NH3a |
| 19 | S233A | 52 | 77 | 102 | 27* | |
| 20 | N27T/G29S/T126N/ | 53 | 78 | 103 | 28** | NHal1 |
| | G143S/N157D/ | | | | (N27T/G29S/ | |
| | S233A | | | | T126N/G143S) | |
| 21 | G25T/G29S/T126N/ | 54 | 79 | 104 | 29** | NHAl2 |
| | G143S/N157D/ | | | | (G25T/G29S/ | |
| | S233A | | | | T126N/G143S) | |
| 22 | G25T/N27S/G29S/ | 55 | 80 | 105 | 30** | NHAl3 |
| | T126N/G143S/ | | | | (G25T/N27S/ | |
| | N157D/S233A | | | | G29S/T126N/ | |
| | | | | | G143S) | |
| 23 | G25T/G29S/T126N/ | 56 | 81 | 106 | 31** | NHAl4 |
| | G143S/S233A | | | | (G25T/G29S/ | |
| | | | | | T126N/G143S) | |
| 24 | G25T/G29S/G56S/ | 57 | 82 | 107 | 32** | NHAl5 |
| | T126N/G143S/ | | | | (G25T/G28S/ | |
| | S233A | | | | G56S/T126N/ | |
| | | | | | G143S) | |
| 25 | G25T/G28S/G56S/ | 58 | 83 | 108 | 33** | NHAl9 |
| | G62S/T126N/ | | | | (G25T/G29S/ | |
| | G143S/S233A | | | | G56S/G62S/ | |
| | | | | | T126N/G143S) | |

*non-catalytic domain;
**catalytic domain only

The present specification further provides for, and includes, lysostaphin variants that are fusion peptides. Fusion peptides can provide for increased plasma half-life. In an aspect, the lysostaphin variants can be fused with polyethylene glycol (PEG). In another aspect, the lysostaphin variants can be fused with the albumin-binding domain (Lst-ABD) of Streptococcal protein G. In another aspect, the lysostaphin variants can be fused with an antibody. In an aspect, the antibody is the Fc effector segment. In aspects, the fusion with an Fc effector segment to prepare a lysibody and in addition to the antimicrobial effects of the lysostaphin variant, provides for the complement fixation and stimulation of phagocytosis of staphylococcal strains.

The variants of the present specification can include additional mutations. In aspects, the lysostaphin variants disclose herein comprise additional mutations known in the art, including the mutations of N12G, N13H, I41E, K46H, L83M, Y93H, K95E, I99Q, R118T, N121G, and S124G of Blazanovic et al. Also included are mutations reported by Shen et al. that reduce glycosylation, N125Q, N232Q, S126P, T127A, and combinations thereof. See Shen et al., “Design and High Expression of Non-glycosylated Lysostaphins in Pichia pastoris and Their Pharmacodynamic Study,” Front. Microbiol. 12:637662 (2021).

### Compositions

The present specification provides for and includes compositions for use in antimicrobial treatment methods, including for medical and veterinary use. In aspects, lysostaphin variant compositions comprise a lysostaphin variant, a diluent, an excipient, a carrier, and combinations thereof. In an aspect, the composition is a pharmaceutical composition. In aspects, the pharmaceutical composition is a veterinary composition. In aspects, the pharmaceutical composition comprises one or more carriers selected from the group consisting of saline, glucose, alcohols, glycols, esters, amides, polymers, and a combination thereof.

The compositions can comprise any of the variants described above. In aspects, the variants comprise the polypeptides having the amino acid sequences of SEQ ID NOs:4, 5, and 9 to 108. Also provided for, and included in the present specification, are compositions that further comprise a one or more additional anti-microbial agents. Suitable antimicrobial agents for use in combination with the lysostaphin variants of the present specification include antibiotics, antimicrobial peptides (AMPs), biocides, lysins, and antiseptic agents. In aspects, the additional anti-microbial agents are selected to target non-lysostaphin sensitive bacteria that are found in mixed populations.

The agents of the inventions may be adminstered in die form of pharmaceutically acceptable salts. The pharmaceutically acceptable salts of the present specification can be synthesized from the lysostaphin variants that contain a basic and acidic moieties (for example, the amino or carboxy termini and basic or acidic side chains) by conventional chemical methods. Generally, such salts can be prepared by reacting the free acid or base forms of the lysostaphin variants with a stoichiometric amount of the appropriate base or acid. Lists of suitable salts are found in Remington's Pharmaceutical Sciences, 17th ed. Mack Publishing Company. Easton, Pa., US, 1985, p. 1418, the disclosure of which is hereby incorporated by reference: see also Stahl et al. Eds, “Handbook of Pharmaceutical Salts Properties Selection and Use” Verlag Helvetica Chimica Acta and Wiley-VCH. 2002. The phrase “pharmaceutically acceptable” is employed herein to refer to those compounds, materials, compositions, and/or dosage forms which are, within the scope of sound medical judgment, suitable for use in contact with the tissues of human beings or, as the case may be, an animal without excessive toxicity, irritation, allergic response, or other problem or complication, commensurate with a reasonable benefit/risk ratio.

In certain aspects, the present specification includes compositions comprising one or more lysostaphin variants of the present specification and a pharmaceutically acceptable carrier, diluent, or excipient. A pharmaceutically acceptable carrier, diluent or excipient refers to a non-toxic solid, semi-solid or liquid filler, diluent, encapsulating material or formulation auxiliary of any type. Prevention of the action of microorganisms, for example Gram-negative bacteria, may be ensured by the inclusion of various antibacterial and antifungal agents, for example, paraben, chlorobutanol, phenol sorbic acid, and the like. It may also be desirable to include isotonic agents such as sugars, sodium chloride, and the like. In an aspect, the pharmaceutical composition comprising lysostaphin variants comprise one or more salts.

In certain aspects, the compositions are prepared for administration orally, parenterally, intracisternally, intravaginally, intraperitoneally, intrarectally, topically (as by powders, ointments, drops, suppository, or transdermal patch), by inhalation (such as intranasal spray), ocularly (such as intraocularly) or buccally. The term “parenteral” as used herein refers to modes of administration which include intravenous, intramuscular, intraperitoneal, intrasternal, subcutaneous, intradermal, and intraarticular, injection and infusion. Accordingly, in certain aspects, the compositions are formulated for delivery by any of these routes of administration. In aspects, the pharmaceutical compositions comprise a liquid, a soft gel capsule, a capsule, a powder, a gel cap, a chewable tablet, a chewable treat, a suspension, a dispersion, a slurry, a lyophilized solid, or a spray.

In certain aspects, pharmaceutical compositions for parenteral injection comprise pharmaceutically acceptable sterile aqueous or nonaqueous solutions, dispersions, suspensions or emulsions, or sterile powders, for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, β-cyclodextrin, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants. These compositions may also contain adjuvants such as preservative, wetting agents, emulsifying agents, and dispersing agents.

Injectable depot forms include those made by forming microencapsule matrices of the lysostaphin variants in one or more biodegradable polymers such as polylactide-polyglycolide, poly(orthoesters), poly(anhydrides), and (poly)glycols, such as PEG. Depending upon the ratio of lysostaphin variant polypeptide to polymer and the nature of the particular polymer employed, the rate of release of the lysostaphin variants can be controlled. Depot injectable formulations are also prepared by entrapping the lysostaphin variants in liposomes or microemulsions compatible with body tissues.

The injectable formulations may be sterilized, for example, by filtration through a bacterial-retaining filter, or by incorporating sterilizing agents in the form of sterile solid compositions which can be dissolved or dispersed in sterile water or other sterile injectable medium just prior to use.

Topical administration includes administration to the skin or mucosa, including surfaces of the lung and eye. Compositions for topical lung administration, including those for inhalation and intranasal, may involve solutions and suspensions in aqueous and non-aqueous formulations and can be prepared as a dry powder which may be pressurized or non-pressurized. In non-pressurized powder compositions, an active lysostaphin variant in finely divided form may be used in admixture with a larger-sized pharmaceutically acceptable inert carrier comprising particles having a size, for example, of up to 100 micrometers in diameter. Suitable inert carriers include sugars such as lactose.

A further form of topical administration is to the eye. Lysostaphin variants of the invention may be delivered in a pharmaceutically acceptable ophthalmic vehicle, such that the lysostaphin variant is maintained in contact with the ocular surface for a sufficient time period to allow the lysostaphin variant to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material. Alternatively, the lysostaphin variants of the specification may be injected directly into the vitreous and aqueous humour.

Lysostaphin variants of the present specification may also be administered in liposomes or other lipid-based carriers. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present compositions in liposome form can contain, in addition to a lysostaphin variants of the present specification, stabilizers, preservatives, excipients, and the like. In certain embodiments, the lipids comprise phospholipids, including the phosphatidyl cholines (lecithins) and serines, both natural and synthetic. Methods to form liposomes are known in the art.

In aspects, the composition is pharmaceutical composition suitable for veterinary use. In aspect, the pharmaceutical compositions comprise a lysostaphin sequence of SEQ ID NO:5 comprising at least one amino acid mutation selected from the group consisting of G25P, G25S, G25T, N27E, N27T, N27D, N27V, N27S, N27A, G29S, G29T, G56S, G62S, T126N, G143S, and S233A. In aspects, the composition comprises a lysostaphin variant comprising a polypeptide having the amino acid sequence of any one of SEQ ID NOs:9 to 108. In another aspect, the pharmaceutical composition comprises a lysostaphin sequence of SEQ ID NO:4 and comprising at least one amino acid mutation selected from the group consisting of G25P, G25S, G25T, N27E, N27T, N27D, N27V, N27S, N27A, G29S, G29T, G56S, G62S, and T126N. In aspects, the composition comprises a polypeptide having the amino acid sequence of any one of SEQ ID NOs:9 to 22.

### Combinations

The present specification further provides for, and includes, methods and compositions that further comprise a one or more additional anti-microbial agents. Suitable antimicrobial agents for use in combination with the lysostaphin variants of the present specification include antibiotics, antimicrobial peptides (AMPs), biocides, lysins, and antiseptic agents. In aspects, the additional anti-microbial agents are selected to target non-lysostaphin sensitive bacteria that are found in mixed populations. As provided herein, diverse anti-microbial agents may be combined to target various subpopulations in a microbial population.

In aspects, the additional anti-microbial agent is an antibiotic. For example, one or more antibiotic agent(s) may be included in an antimicrobial composition of the present specification, such as, e.g., aminoglycosides, beta lactams, quinolones or fluoroquinolones, macrolides, sulfonamides, sulfamethaxozoles, tetracyclines, streptogramins, oxazolidinones (such as linezolid), clindamycins, lincomycins, rifamycins, glycopeptides, polymxins, lipo-peptide antibiotics, as well as pharmacologically acceptable sodium salts, pharmacologically acceptable calcium salts, pharmacologically acceptable potassium salts, lipid formulations, derivatives and/or analogs of the above. The antibiotic may be a member of the penicillin group of antibiotics such as, e.g., amoxicillin, ampicillin, benzathine penicillin G, carbenicillin, cloxacillin, dicloxacillin, piperacillin, or ticarcillin, etc. Examples of cephalosporins include ceftiofur, ceftiofur sodium, cefazolin, cefaclor, ceftibuten, ceftizoxime, cefoperazone, cefuroxime, cefprozil, ceftazidime, cefotaxime, cefadroxil, cephalexin, cefamandole, cefepime, cefdinir, cefriaxone, cefixime, cefpodoximeproxetil, cephapirin, cefoxitin, cefotetan etc. Examples of beta lactamase inhibitors include clavulanate, sulbactam, or tazobactam. The antibiotic may be a macrolide such as, e.g., erythromycin, azithromycin, or claritbromycin. Examples of quinolones and fluoroquinolones that may be used include nalidixic acid, cinoxacin, trovafloxacin, ofloxacin, levofloxacin, grepafloxacin, trovafloxacin, sparfloxacin, norfloxacin, ciprofloxacin, moxifloxacin and gatifloxacin. Examples of sulphonamides that may be used include mafenide, sulfisoxazole, sulfamethoxazole, and sulfadiazine. The streptogramin class of antibacterial agents is exemplified by quinupristin, dalfopristin or the combination of two streptogramins.

Certain lysostaphin-antibiotic combinations are known to generate synergistic anti-staphylococcal effects. In aspects, the lysostaphin variant compositions further comprise an antibiotic selected from the group consisting of aminoglycosides, ansaycins, carbapenems, cephalosporins, glycopeptides, lincosamides, lipopeptides, macrolides, monobactams, nitrofurans, oxazolidinones, penicillins, polypeptides, quinolones/fluoroquinolone, sulfonamides, tetracyclines, clofazimine, dapsone, capreomycin, cycloserine, ethambutol, ethionamide, isoniazid, pyrazinamide, rifampicin (rifampin), rifabutin, rifapentine, streptomycin, arsphenamine, chloramphenicol, fosfomycin, fusidic acid, metronidazole, mupirocin, platensimycin, quinupristin/dalfopristin, thiamphenicol, tigecycline, tinidazole, and trimethoprim, and combinations thereof, Notably, in vitro synergism has been demonstrated with such antibiotics as cefazolin, clarithromycin, doxycycline, levofloxacin, linezolid, rifampicin, teicoplanin, vancomycin, quinupristin, dalfopristin, bacitracin, polymyxin B, daptomycin, gentamycin, tetracycline and erythromycin, demonstrated in vitro synergism against S. aureus. See Jayakumar et al. In aspects, the combination comprises vancomycin, methicillin, nafcillin, mupirocin, or neomycin.

In other aspects, the lysostaphin variants of the present specification can be combined in compositions with antimicrobial peptides (AMPs), including but not limited to, Dermaseptin-S3, KR-20, Magainin-2, Nigrocin-OG13, Pexiganan, MUC7-12mer, Ranalexin, Gomesin, Melanotropin-a, hLF (1-11), Parasin-I, Odorranain-G1, Nisin, Colistin, Daptomycin, Polymyxin B and Bovine lactoferrin. See Graham and Coote 2007 and Desbois and Coote 2011. The combination of lysostaphin with AMPs is known to provide synergistic effects against staphylococcus.

In aspects, the antimicrobial agent for use in combination with the lysostaphin variant compositions and methods provided herein may further comprise one or more additional antimicrobial agents. The one or more additional antimicrobial agents may be an antibiotic, an antiseptic, chlorhexidine, gendine, gardine, silver, nanosilver, silver sulfadiazine, polyhexamethylene biguanide (PHMB), a chelator, a C₁₋₄ alcohol, a nitric oxide donor, a quaternary ammonium antimicrobial, or mixtures thereof. In some aspects, the lysostaphin variant compositions further comprise one or more nitroprussides, nitrosoglutathione, other nitroso compounds, nitrosothiols, nitrosocystein, nitrosoalbumin, nitro compounds, nitroaspiririns, isosorbides, diazeniumdiolates, nitrates and nitrites, a glyceryl nitrate, such as glyceryl trinitrate, and/or dilute hydrogen peroxide (e.g., less than about 1%, or 0.1, 0.2, 0.3, 0.4, 0.5, 0.6, 0.7, 0.8, 0.9% or any range derivable therein).

The lysostaphin variant compositions and methods provided herein may further comprises an antiseptic agent Several antiseptic agents are: known in the art and these include a taurinamide derivative, a phenol, a quaternary ammonium surfactant, a chlorine-containing agent, a quinaldinium, a lactone, a dye, a thiosemicarbazone, a quinone, a carbamate, urea, salicylamide, carbanilide, a guanide, an amidine, an imidazoline biocide, acetic acid, benzoic acid, sorbic acid, propionic acid, boric acid, dehydroacetic acid, sulfurous acid, vanillic acid, esters of p-hydroxybenzoic acid, isopropanol, propylene glycol, benzyl alcohol, chlorobutanol, phenylethyl alcohol, 2-bromo-2-nitropropan-1,3-diol, formaldehyde, glutaraldehyde, calcium hypochlorite, potassium hypochlorite, sodium hypochlorite, iodine (in various solvents), povidone-iodine, hexamethylenetetramine, noxythiolin 1-(3-chloroallyl)-3,5,7-triazo 1-azoniaadamantane chloride, taurolidine, taurultam, N(5-nitro-2-furfurylidene)-1-amino-hydantoin, 5-nitro-2-furaldehyde semicarbazone, 3,4,4′-trichlorocarbanilide, 3,4′,5 tribromosalicylanilide, 3-trifluoromethyl-4,4′-trichlorocarbanilide, 8-hydroxyquinoline, 1-cyclopropyl-6-fluoro-1,4-dihydro-4-oxo-7-(1-piperazinyl)-3-quinolinecarboxylic acid, 1,4-dihydro-1-ethyl-b6-fluoro-4-oxo-7-(1-piperazinyl)-3-quinolinecarboxylic acid, hydrogen peroxide, peracetic acid, phenol, sodium oxychlorosene, parachlorometaxylenol, 2,4,4′-trichloro-2′-hydroxydiphenol, thymol, chlorhexidine, benzalkonium chloride, cetylpyridinium chloride, silver sulfadiazine, or silver nitrate.

Also included and provided for in the present specification are lysostaphin variant compositions and methods that further comprise an enzyme-based antibiotic (“enzybiotics”) such as bacteriophage lysins. Like lysostaphins, the bacteriophage derived lysins are enzymes that digest the cell wall of their bacterial hosts, releasing viral progeny through hypotonic lysis. A similar outcome results when purified, recombinant lysins are added externally to Gram-positive bacteria. The high lethal activity of lysins against Gram-positive pathogens makes them attractive candidates for development as therapeutics in combination with the lysostaphin variants provided herein. Bacteriophage lysins were initially proposed for eradicating the nasopharyngeal carriage of pathogenic streptococci. See Schuch et al., “Direct Lytic Agents: Novel, Rapidly Acting Potential Antimicrobial Treatment Modalities for Systemic Use in the Era of Rising Antibiotic Resistance,” Front. Microbiol. 13:841905 (2022). Lysins are part of the lytic mechanism used by double stranded DNA (dsDNA) phage to coordinate host lysis with completion of viral assembly (Wang et al., “Holins: the protein clocks of bacteriophage infections,” Ann. Rev. Microbiol. 54:799-825 (2000). Phage encode both holins that open a pore in the bacterial membrane, and peptidoglycan hydrolases called lysins that break bonds in the bacterial wall. Late in infection, lysin translocates into the cell wall matrix where it rapidly hydrolyzes covalent bonds essential for peptidoglycan integrity, causing bacterial lysis and concomitant progeny phage release. Examples of lytic enzymes that cleave these bonds are various amidases such as muramidases, glucosaminidases, endopeptidases, or N-acetyl-muramoyl-L-alanine amidases. Fischetti and co-workers reported that the C1 streptococcal phage lysin enzyme was an amidase. See Nelson et al., “PlyC: A multimeric bacteriophage lysin,” PNAS 103(28)10765-10770 (2006). Garcia et al., “Cloning, purification, and biochemical characterization of the pneumococcal bacteriophage Cp-1 lysin,” J. Virol. 61(8):2573-80 (1987) report that the Cp1 lysin from a S. pneumoniae from a Cp-1 phage was a lysozyme. Caldentey and Bamford report a lytic endopeptidase enzyme from the phi 6 Pseudomonas phage that splits the peptide bridge formed by melo-diaminopimilic acid and D-alanine. See Caldenty and Bamford, “The lytic enzyme of the Pseudomonas phage phi 6. Purification and biochemical characterization,” Biochim. Biophys. Acta. 1159(1)44-50 (1992). The E. coli T1 and T6 phage lytic enzymes are amidases as is the lytic enzyme from Listeria phage (ply) are also suitable for use in combination with the lysostaphin variants of the present application. See Loessner et al., “Modified Listeria bacteriophage lysin genes (ply) allow efficient overexpression and one-step purification of biochemically active fusion proteins,” Appl. Environ Microbiol 62(8):3057-60 (1996). Other lytic enzymes known in the art that are capable of cleaving a bacterial cell wall are suitable for use in lysostaphin variant compositions and methods of the present specification.

### Nucleic Acids and Expression

A person skilled in the art will appreciate that the lysostaphin variant proteins provided herein may be prepared in any of several ways, including, without limitation, by using recombinant methods.

Accordingly, nucleic acid molecules encoding the lysostaphin variants may be incorporated in a known manner into an appropriate expression vector which ensures good expression of the proteins. Possible expression vectors include but are not limited to cosmids, plasmids or modified viruses (e.g. replication defective retroviruses, adenoviruses and adeno-associated viruses), so long as the vector is compatible with the host cell used. The expression vectors are “suitable for transformation of a host cell”, which means that the expression vectors contain a nucleic acid molecule of the application and regulatory sequences selected on the basis of the host cells to be used for expression, which is operatively linked to the nucleic acid molecule. Operatively linked is intended to mean that the nucleic acid is linked to regulatory sequences in a manner that allows expression of the nucleic acid.

The specification therefore contemplates a recombinant expression vector containing a nucleic acid molecule encoding a lysostaphin variant disclosed herein, and the necessary regulatory sequences for the transcription and translation of the inserted protein-sequence. Exemplary nucleic acid sequences encoding lysostaphin variant polypeptides may be selected from the group consisting of SEQ ID NOs: 110 to 127. As is known to persons of skill in the art, modifications of the nucleic acids can be prepared as appropriate based on the intended host to adjust for codon usage and to optimize expression. In a further aspect, at least one lysostaphin variant may be expressed by a vector and the methods or uses provided herein comprise use of or administration of the vector to the animal in need thereof.

Suitable regulatory sequences may be derived from a variety of sources, including bacterial, fungal, viral, mammalian, or insect genes (For example, see the regulatory sequences described in Goeddel, Gene Expression Technology: Methods in Enzymology 185, Academic Press. San Diego. Calif. (1990)). Selection of appropriate regulatory sequences is dependent on the host cell chosen as discussed below, and may be readily accomplished by one of ordinary skill in the art. Examples of such regulatory sequences include: a transcriptional promoter and enhancer or RNA polymerase binding sequence, a ribosomal binding sequence, including a translation initiation signal. Additionally, depending on the host cell chosen and the vector employed, other sequences, such as an origin of replication, additional DNA restriction sites, enhancers, and sequences conferring inducibility of transcription may be incorporated into the expression vector.

The recombinant expression vectors of the specification may also contain a selectable marker gene which facilitates the selection of host cells transformed or transfected with a recombinant molecule of the application.

The recombinant expression vectors may also contain genes which encode a fusion moiety which provides increased expression of the recombinant protein; increased solubility of the recombinant protein; and aid in the purification of the target recombinant protein by acting as a ligand in affinity purification. For example, a proteolytic cleavage site may be added to the target recombinant protein to allow separation of the recombinant protein from the fusion moiety subsequent to purification of the fusion protein. Typical fusion expression vectors include pGEX (Amrad Corp. Melbourne, Australia), pMal (New England Biolabs, Beverly, Mass.) and pIT5 (Pharmacia, Piscataway, N.J.) which fuse glutathione S-transferase (GST), maltose E binding protein, or protein A, respectively, to the recombinant protein.

In aspects, the present specification provides for a vector comprising a transcription promoter, a nucleic acid sequence encoding a lysostaphin polypeptide sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A, and a transcriptional terminator. The present specification further provides for a recombinant nucleic acids and vectors thereof, for the expression of the lysostaphin variants of SEQ ID NOs:4, 5, and 9 to 108.

In an aspect, the vector is a pET vector (see U.S. Pat. No. 4,952,496). Other suitable expression systems are known in the art and commercially available.

Recombinant expression vectors can be introduced into host cells to produce a transformed host cell. The terms “transformed with”. “transfected with”, “transformation” and “transfection” are intended to encompass introduction of nucleic acid (e.g. a vector) into a cell by one of many possible techniques known in the art. Suitable methods for transforming and transfecting host cells are well known in the art.

Suitable host cells include eukaryotic host cells and prokaryotic cells. For example, host cell can be selected from a bacterium or yeast. In aspects, the host cell is selected from E. coli, B. subtilis, or a Lactobacillus species. Methods of gene expression are well known in the art. See Gene expression technology. Methods Enzymol. 1990:185:1-611. PMID: 2199770. In aspects, the proteins of the specification are expressed in prokaryotic cells, such as Escherichia coli (Zhang et al., Science 303(5656): 371-3 (2004)).

The present specification provides for, and includes, method of producing a lysostaphin variant comprising a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A, said method comprising inducing expression of the lysostaphin polypeptide from the recombinant nucleic acid vector in a cell. In another aspect, a method of producing a lysostaphin variant comprising a lysostaphin sequence of SEQ ID NO:4 and comprising at least one amino acid mutation selected from the group consisting of G25P, G25S, G25T, N27E, N27T, N27D, N27V, N27S, N27A, G29S, G29T, G56S, G62S, and T126N, said method comprising inducing expression of the lysostaphin polypeptide from the recombinant nucleic acid vector in a cell. In aspects, the method provides for the producing an lysostaphin variant having the amino acid sequence of any one of SEQ ID NOs:9 to 108.

### Methods of Treatment

The present specification provides for, and includes, methods of treatment using the lysostaphin variants disclosed herein, and their pharmaceutical compositions.

In an aspect, a method of treating an infection in an animal is provided. The method comprises the step of administering a pharmaceutical composition or a veterinary composition as described herein to an animal, wherein the pharmaceutical composition or the veterinary composition reduces one or more symptoms associated with the infection in the animal.

In an aspect, the methods comprise preparing a pharmaceutical composition for intravenous, intraarterial, intraperitoneal, intrathecal, intradermal, epidural, intracerebroventricular, intraurethral, intrasternal, intracranial, intratumoral, intramuscular or subcutaneous administration.

The methods further provide for, and include, treatment with one or more additional therapeutic agents. In some aspects, the lysostaphin variant compositions further comprise a one or more additional anti-microbial agents for use in combination methods. In other aspects, the one or more therapeutic agents can be provided as a separate composition. In aspects, the methods of treatment comprise providing a lysostaphin variant composition comprising a lysostaphin variant, a diluent, an excipient, a carrier, and combinations thereof. Also provided for, and included in the present specification, are methods comprising providing a lysostaphin variant composition comprising one or more additional anti-microbial agents as provided above for the treatment of microbial infections, contaminations, biofilms, and the like. Suitable antimicrobial agents for use in combination with the lysostaphin variants of the present specification include antibiotics, antimicrobial peptides (AMPs), biocides, lysins, and antiseptic agents. In aspects, the additional anti-microbial agents are selected to target non-lysostaphin sensitive microbes that are found in mixed populations.

The present specification provides for a method of treatment of a bacterial infection, contamination, or biofilm, comprising providing an effective amount of a lysostaphin variant in combination with a second agent selected from the group consisting of cefazolin, clarithromycin, doxycycline, levofloxacin, linezolid, rifampicin, teicoplanin, vancomycin, quinupristin, dalfopristin, bacitracin, polymyxin B, daptomycin, gentamycin, tetracycline, erythromycin, and combinations thereof. In aspects, the combination therapy comprises a combination therapy with vancomycin, methicillin, nafcillin, mupirocin, neomycin, or a combination thereof. As provided herein, the one or more additional therapeutic agents can be provided in a single combination composition or provided separately.

In other aspects, the methods provide for treatments and applications of lysostaphin variants of the present specification in combination therapies with antimicrobial peptides (AMPs). In an aspect the combination treatment comprises a lysostaphin variant composition combined with an AMP selected from the group consisting of Dermaseptin-S3, KR-20, Magainin-2, Nigrocin-OG13, Pexiganan, MUC7-12mer, Ranalexin, Gomesin, Melanotropin-a, hLF (1-11), Parasin-I, Odorranain-G1, Nisin, Colistin, Daptomycin, Polymyxin B and Bovine lactoferrin.

As provided herein, the methods for treating an infection in an animal includes treatment of nasal decolonization and for the treatment of wounds, keratitis, endophthalmitis, orthopedic, endocarditis and implant-associated biofilm infections. In aspects, the nasal cavity is colonized by S. aureus and treated using a petrolatum-based cream formulation or by a nasal spray formulation. Methods of preparing suitable nasal formulations are known in the art and as provided above.

The compositions and methods also provide for, and include, the treatment of soft tissue infections. In aspects, the soft tissue infection is an infection of a burn, ulcer, or surgery site wound. In aspects, the lysostaphin variants are prepared for topical application, for example as a cream. In other aspects, the lysostaphin variants can be incorporated into bandages and wound dressings. In aspects, the lysostaphin variants combined with one or more second agents as provided above.

In an aspect, the compositions and methods provide for the treatment of keratitis (inflammation of the cornea) and post-surgical endophthalmitis (inflammation of the intraocular fluids). The lysostaphin variants provided herein can be applied topically or by intravitreal injection.

In another aspect, the compositions and methods provide for the treatment of infections of orthopedic implants. In an aspect, the lysostaphin variants can be incorporated into control released, self-setting and injectable porous bone cement using hydroxyapatite/chitosan (HA/CS) composite scaffold. See Xue et al. The lysostaphin variant containing HA/CS compositions can be used to treat, for example, S. aureus-infected bone defects and infections Xue et al., “A novel controlled-release system for antibacterial enzyme lysostaphin delivery using hydroxyapatite/chitosan composite bone cement,” PLoS One 9(12):e113797 (2014). In other aspects, lysostaphin can be coated on medical devices suitable for implantation in an animal or patient in need thereof. Lysostaphins are known to be suitable for application on plastic, glass, or metal surfaces.

The present specification provides for methods of treatment of surfaces to prevent, reduce, or eliminate biofilms. See Suresh et al., “An update on recent developments in the prevention and treatment of Staphylococcus aureus biofilms,” Int J Med Microbiol. 309(1):1-12 (2019). Notably, biofilms are a significant concern and S. aureus biofilms are known to be resistant to antibiotics. In aspects, the lysostaphin variants of the present specification are suitable for the prevention, reduction, or elimination, of biofilms, for example on medical device surfaces. In an aspect, the present specification provides for the treatment of catheters. In aspects, the activity of the lysostaphin variants of the present specification is maintained for at least 4 days. In aspects, the lysostaphin variants are applied to the surface every day, every other day, or every three days. Importantly, as lysostaphin is known to be non-toxic, the lysostaphin variants can be used for the treatment of medical devices to reduce biofilms and used directly in the animal.

Infections by other gram-positive bacteria can be treated using the compositions, formulations, and methods, provided herein. More specifically, the lysostaphin variants provided herein can be used in methods of combination therapy with agents that target gram-positive bacteria. In other aspects, the methods provide for using lysostaphin variants to treat mixed populations of gram-positive and gram-negative bacteria. Not to be limited by theory, mixed bacterial populations containing gram-positive bacteria such as S. aureus can render non-gram-positive microbes to be refractory to treatment. By targeting the S. aureus with lysostaphin variants, second anti-microbial agents can then access the other microbes in the population and provide for effective treatment.

The present specification provides for, and includes methods of treating medical devices. Also included are treatment of surfaces in need of reductions in bacterial growth, and prevention of biofilms. In an aspect, the methods include a method of treating colonization of medical devices comprising applying a lysostaphin polypeptide variant to the surface of a medical device. In certain aspects, the surface is a plastic, glass, metal surface. In aspects provided herein, the treatment is the reduction of surface colonization by S. aureus or S. epidermidis.

In an aspect, the present specification provides for methods of treatment of Staphylococcus blood infection, said method comprising administering the lysostaphin variant or pharmaceutical composition thereof intraperitoneally or subcutaneously. In another aspect, the present compositions and methods provide for the treatment of Staphylococcus infected abscess lesions comprising contacting the infected abscess lesion with a lysostaphin variant containing composition. In an aspect, the abscess lesion is treated with a topical composition. In other aspect, the abscess lesion is treated by an injectable composition as described above.

In an aspect, the present specification provides for methods of treatment of infections, wherein the infection is Staphylococcus endocarditis and the method comprises administering the lysostaphin variant or pharmaceutical composition thereof intravenously. In other aspects, the endocarditis may be caused by other Gram-positive bacteria.

Included and provided for by the present specification are methods of treatment of bacterial infections or contaminations wherein the Staphylococcus bacterium is Staphylococcus aureus, Staphylococcus pseudintermedius, or Staphylococcus agnetis. In other aspects, the Staphylococcus bacterium is a Methicillin Resistant Staphylococcus aureus (MRSA) bacterium, a Vancomycin Intermediate Staphylococcus aureus (VISA) bacterium, or a Vancomycin Resistant Staphylococcus aureus (VRSA) bacterium.

Included and provided for by the present specification are methods of treatment of bacterial infections or contaminations wherein the infection is caused by a Streptococcus bacterium. In aspects, the Streptococcus bacterium is Streptococcus suis.

The present specification provides for methods of treatment of animals. In aspects, the animal is a human patient in need of treatment. In other aspects, the animal is selected from the group consisting of humans, ruminant animals, poultry, swine, canines, felines, Mustelidae family, and Leporidae family.

The specification further provides for the treatment of ruminant animals that are members of the Bovinae subfamily. In aspects, the member of the Bovinae subfamily is an animal of the genus Bubalus, Bos, or Bison. The specification further provides for the treatment of poultry. In aspects, the poultry is selected from the group of ducks, geese, chickens, chickens, laying hens, broilers, turkeys, quails, ostriches, and turkeys. In aspects, the animal for treatment using the present methods are swine. In an aspect, the swine is selected from the group consisting of pigs, boars, sows, gilts, and piglets.

The methods of treatment provide for, and include, providing an effective amount of the lysostaphin variants either alone or in combination with another therapeutic agent. As provided herein, the lysostaphin variant or pharmaceutical composition containing the lysostaphin variant, or combination of lysostaphin variant and a second agent is administered at an amount of between 0.001 to 1000 mg of the lysostaphin variant. In another aspect, the lysostaphin variant or pharmaceutical composition is administered at a dose of about 0.001 to about 1000 mg of the lysostaphin variant per kg of animal body weight.

The methods of the present specification provide for a variety of suitable dosage forms, the selection of which depends on the nature and type of infection, and the selection of the dosage form and composition are known to those skilled in the art. In aspects, the lysostaphin variant or pharmaceutical composition is an oral formulation selected from the group consisting of a tablet, a capsule, a suspension, an emulsion, a syrup, a colloidal dispersion, a dispersion, and an effervescent composition.

The present specification provides for, and includes, parental formulations. In aspects, a parenteral formulation is selected from the group consisting of intravenous, intraarterial, intraperitoneal, intrathecal, intradermal, epidural, intracerebroventricular, intraurethral, intrasternal, intracranial, intratumoral, intramuscular and subcutaneous formulations.

Also included are lysostaphin variant or compositions as provided as an animal feed or animal feed supplement.

In an aspect, the method of treating an infection in an animal comprises providing a lysostaphin variant or pharmaceutical composition comprises a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A. In an aspect, the lysostaphin variant comprises the amino acid mutations N27T, G29S, T126N, G143S, N157D, and S233A. In aspects, the lysostaphin variant comprises a polypeptide having the amino acid sequence of any one of SEQ ID NOs:9 to 108.

The specification provides for methods of treating animals the method comprising the steps of administering a lysostaphin variant, a composition comprising a lysostaphin variant, wherein the composition reduces the levels of Gram-positive bacterium in said animal. In aspects, the composition is a pharmaceutical composition or a veterinary composition. In an aspect, the Gram-positive bacterium is a Staphylococcus bacterium or a Streptococcus bacterium.

In aspects, the method provides for the treatment of animals selected from the group consisting of humans, ruminant animals, poultry, swine, canines, felines, Mustelidae family, and Leporidae family. In an aspect, the animal is a canine and the treatment is an oral treatment.

The specification further provides for methods of treating mastitis in an animal, the method comprising providing a lysostaphin variant or composition thereof, to an animal having mastitis wherein the lysostaphin variant is formulated for topical administration or injection.

### Embodiments

The following numbered embodiments are contemplated and are non-limiting:
1. A lysostaphin variant comprising a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A.
2. The lysostaphin variant of embodiment 1, wherein the lysostaphin variant has a lower amount of degradation compared to wild type lysostaphin.
3. The lysostaphin variant of embodiment 1 or 2, wherein the lysostaphin variant comprises at least 2 amino acid mutations.
4. The lysostaphin variant of embodiment 1 or 2, wherein the lysostaphin variant comprises at least 3 amino acid mutations.
5. The lysostaphin variant of embodiment 1 or 2, wherein the lysostaphin variant comprises at least 4 amino acid mutations.
6. The lysostaphin variant of embodiment 1 or 2, wherein the lysostaphin variant comprises at least 5 amino acid mutations.
7. The lysostaphin variant of embodiment 1 or 2, wherein the lysostaphin variant comprises at least 6 amino acid mutations.
8. The lysostaphin variant of any one of embodiments 1, 2, or 7, wherein the lysostaphin variant comprises the amino acid mutations N27T, G29S, T126N, G143S, N157D, and S233A.
9. The lysostaphin variant of any one of embodiments 1 to 8, further comprising a polypeptide having the amino acid sequence of SEQ ID NO:6 joined via a peptide bond between the carboxy terminus of SEQ ID NO:6 and the amino terminus of SEQ ID NO:5.
10. The lysostaphin variant of any one of embodiments 1 to 8, further comprising a polypeptide having the amino acid sequence of SEQ ID NO:7 joined via a peptide bond between the carboxy terminus of SEQ ID NO:7 and the amino terminus of SEQ ID NO:5.
11. The lysostaphin variant of any of embodiments 1 to 10, wherein said variant comprises a polypeptide having the amino acid sequence of any one of SEQ ID NOs:9 to 108.
12. The lysostaphin variant of any of embodiments 1 to 11, wherein said variant further comprises one or more mutations selected from the group consisting of N12G, N13H, I41E, K46H, L83M, Y93H, K95E, I99Q, R118T, N121G, and S124G.
13. A lysostaphin variant comprising a lysostaphin sequence of SEQ ID NO:4 and comprising at least one amino acid mutation selected from the group consisting of G25P, G25S, G25T, N27E, N27T, N27D, N27V, N27S, N27A, G29S, G29T, G56S, G62S, and T126N.
14. The lysostaphin variant of embodiment 13, wherein the lysostaphin variant has a lower amount of degradation compared to wild type lysostaphin.
15. The lysostaphin variant of embodiment 13 or 14, wherein said lysostaphin variant comprises a polypeptide having the amino acid sequence of any one of SEQ ID NOs:9 to 22.
16. The lysostaphin variant of embodiment 15, further comprising a serine at position 143.
17. The lysostaphin variant of embodiment 16, wherein said lysostaphin variant comprises a polypeptide having the amino acid sequence of any one of SEQ ID NOs:28 to 33.
18. The lysostaphin variant of any one of embodiments 13 to 16, further comprising a polypeptide having the amino acid sequence of SEQ ID NO:6 joined via a peptide bond between the carboxy terminus of SEQ ID NO:6 and the amino terminus of SEQ ID NO:4.
19. The lysostaphin variant of embodiment 18, wherein said lysostaphin variant comprises a polypeptide having the amino acid sequence of any one of SEQ ID NOs:59 to 72.
20. The lysostaphin variant of any one of embodiments 13 to 16, further comprising a polypeptide having the amino acid sequence of SEQ ID NO:8 joined via a peptide bond between the amino terminus of SEQ ID NO:8 and the carboxy terminus of SEQ ID NO:4.
21. The lysostaphin variant of any one of embodiments 13 to 16, further comprising a polypeptide having the amino acid sequence of SEQ ID NO:8 comprising one or more amino acid mutations selected from the group consisting of G1S, G2Q, N15D, K16Q, and S91A, joined via a peptide bond between the amino terminus of SEQ ID NO:8 and the carboxy terminus of SEQ ID NO:4.
22. The lysostaphin variant of embodiment 21, wherein said polypeptide having the amino acid sequence of SEQ ID NO:8 comprises a polypeptide having the amino acid sequence of any one of SEQ ID NOs:23 to 27.
23. The lysostaphin variant of embodiment 20, wherein said lysostaphin variant comprises a polypeptide having the amino acid sequence of any one of SEQ ID NOs:73 to 83.
24. The lysostaphin variant of any one of embodiments 13 to 16, further comprising a polypeptide having the amino acid sequence of SEQ ID NO:7 joined via a peptide bond between the carboxy terminus of SEQ ID NO:7 and the amino terminus of SEQ ID NO:4
25. The lysostaphin variant of embodiment 24, wherein said lysostaphin variant comprises a polypeptide having the amino acid sequence of any one of SEQ ID NOs:84 to 97.
26. The lysostaphin variant of embodiment 24, further comprising a polypeptide having the amino acid sequence of SEQ ID NO:8 comprising one or more amino acid mutations selected from the group consisting of G1S, G2Q, N15D, K16Q, and S91A.
27. The lysostaphin variant of any one of embodiments 13 to 16, further comprising SEQ ID NO:6 at the amino terminal end, and SEQ ID NO:8 at the carboxy terminal end and wherein said lysostaphin variant comprises a polypeptide having the amino acid sequence of any one of SEQ ID NOs:98 to 108.
28. The lysostaphin variant of any one of embodiments 1 to 27, wherein the degradation is self-degradation.
29. The lysostaphin variant of any one of embodiments 1 to 28, wherein the degradation is reduced during fermentation of the lysostaphin or the lysostaphin variant compared to wild type lysostaphin.
30. The lysostaphin variant of any one of embodiments 1 to 29, wherein the degradation is reduced at room temperature compared to wild type lysostaphin.
31. The lysostaphin variant of any one of embodiments 1 to 29, wherein the degradation is reduced at 20° C. compared to wild type lysostaphin.
32. The lysostaphin variant of any one of embodiments 1 to 29, wherein the degradation is reduced at 37° C. compared to wild type lysostaphin.
33. A pharmaceutical composition comprising the lysostaphin variant of any of embodiments 1-32.
34. The pharmaceutical composition of embodiment 33, wherein said pharmaceutical composition comprises one or more carriers selected from the group consisting of saline, glucose, alcohols, glycols, esters, amides, polymers, and a combination thereof.
35. The pharmaceutical composition of embodiment 33 or 34, wherein said pharmaceutical composition comprises one or more salts.
36. The pharmaceutical composition of any one of embodiments 33 to 35, wherein the composition comprises a liquid, a soft gel capsule, a capsule, a powder, a gel cap, a chewable tablet, a chewable treat, a suspension, a dispersion, a slurry, a lyophilized solid, or a spray.
37. The pharmaceutical composition of any one of embodiments 33 to 36, wherein the carrier comprises a surfactant.
38. The pharmaceutical composition of any one of embodiments 33 to 37, wherein the pharmaceutical composition comprises a further active pharmaceutical ingredient (API).
39. The pharmaceutical composition of any one of embodiments 33 to 38, wherein the pharmaceutical composition comprises a second therapeutic agent.
40. The pharmaceutical composition of any one of embodiments 33 to 39, wherein the pharmaceutical composition is a veterinary composition.
41. A pharmaceutical composition comprising a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A.
42. The pharmaceutical composition of embodiment 41, wherein said lysostaphin variant comprises a polypeptide having the amino acid sequence of any one of SEQ ID NOs:9 to 108.
43. The pharmaceutical composition of embodiment 33, wherein said lysostaphin variant comprises a lysostaphin sequence of SEQ ID NO:4 and comprising at least one amino acid mutation selected from the group consisting of G25P, G25S, G25T, N27E, N27T, N27D, N27V, N27S, N27A, G29S, G29T, G56S, G62S, and T126N.
44. The pharmaceutical composition of embodiment 43, wherein said lysostaphin variant comprises a polypeptide having the amino acid sequence of any one of SEQ ID NOs:9 to 22.
45. The pharmaceutical composition of any one of embodiments 41, wherein said pharmaceutical composition comprises one or more carriers selected from the group consisting of saline, glucose, alcohols, glycols, esters, amides, polymers, and a combination thereof.
46. The pharmaceutical composition of any one of embodiments 41, wherein said pharmaceutical composition comprises one or more salts.
47. The pharmaceutical composition of any one of embodiments 41, wherein the composition comprises a liquid, a soft gel capsule, a capsule, a powder, a gel cap, a chewable tablet, a chewable treat, a suspension, a dispersion, a slurry, a lyophilized solid, or a spray.
48. The pharmaceutical composition of any one of embodiments 41, wherein the carrier comprises a surfactant.
49. The pharmaceutical composition of any one of embodiments 41, wherein the pharmaceutical composition comprises a further active pharmaceutical ingredient (API).
50. The pharmaceutical composition of any one of embodiments 41, wherein the pharmaceutical composition comprises a second therapeutic agent.
51. The pharmaceutical composition of any one of embodiments 41, wherein the pharmaceutical composition is a veterinary composition.
52. A method of treating an infection in an animal, said method comprising the steps of administering the lysostaphin variant of any one of embodiments 1 to 32, a pharmaceutical composition of any of embodiments 33 to 51, wherein the pharmaceutical composition or the veterinary composition reduces one or more symptoms associated with the infection in the animal.
53. The method of embodiment 52, further comprising administering a second therapeutic agent selected from an antibiotic,
54. The method of embodiment 53, wherein said antibiotic is selected from vancomycin, methicillin, nafcillin, Mupirocin neomycin.
55. The method of embodiment 54, wherein said combination of the lysostaphin variant or pharmaceutical composition thereof and an antibiotic provides synergistic effect.
56. The method of any one of embodiments 52, wherein the infection is an oral cavity infection or a soft tissue infection.
57. The method of any one of embodiments 52 to 55, wherein said infection is an infection of the nasal cavity.
58. The method of embodiment 57, wherein the pharmaceutical composition is a petrolatum-based cream formulated for topical application.
59. The method of any one of embodiments 52, wherein the infection is a Staphylococcus blood infection, said method comprising administering the lysostaphin variant or pharmaceutical composition thereof intraperitoneally or subcutaneously.
60. The method of any one of embodiments 52, wherein the infection is a Staphylococcus infected abscess lesion.
61. The method of any one of embodiments 52, wherein the infection is Staphylococcus endocarditis and said method comprising administering the lysostaphin variant or pharmaceutical composition thereof intravenously.
62. The method of any one of embodiments 52, wherein the infection is caused by a Gram positive bacterium.
63. The method of any one of embodiments 52, wherein the infection is caused by a Staphylococcus bacterium.
64. The method of any one of embodiments 52, wherein the Staphylococcus bacterium is Staphylococcus aureus, Staphylococcus pseudintermedius, or Staphylococcus agnetis.
65. The method of any one of embodiments 52 to 64, wherein said Staphylococcus bacterium is a Methicillin Resistant Staphylococcus aureus (MRSA) bacterium, a Vancomycin Intermediate Staphylococcus aureus (VISA) bacterium, or a Vancomycin Resistant Staphylococcus aureus (VRSA) bacterium.
66. The method of any one of embodiments 52, wherein the infection is caused by a Streptococcus bacterium.
67. The method of embodiment 66, wherein the Streptococcus bacterium is Streptococcus suis.
68. The method of any one of embodiments 52, wherein said animal is selected from the group consisting of humans, ruminant animals, poultry, swine, canines, felines, Mustelidae family, and Leporidae family.
69. The method of embodiment 68, wherein the ruminant animal is a member of the Bovinae subfamily.
70. The method of embodiment 69, wherein the member of the Bovinae subfamily is an animal of the genus Bubalus, Bos, or Bison.
71. The method of embodiment 68, wherein the poultry is selected from the group of ducks, geese, chickens, chickens, laying hens, broilers, turkeys, quails, ostriches, and turkeys.
72. The method of embodiment 68, wherein the animal is a swine.
73. The method of embodiment 72, wherein the swine is selected from the group consisting of pigs, boars, sows, gilts, and piglets.
74. The method of any one of embodiments 52, wherein the lysostaphin variant or pharmaceutical composition is administered at an amount of between 0.001 to 1000 mg of the lysostaphin variant.
75. The method of any one of embodiments 52, wherein the lysostaphin variant or pharmaceutical composition is administered at a dose of about 0.001 to about 1000 mg of the lysostaphin variant per kg of animal body weight.
76. The method of any one of embodiments 52, wherein the lysostaphin variant or pharmaceutical composition is an oral formulation selected from the group consisting of a tablet, a capsule, a suspension, an emulsion, a syrup, a colloidal dispersion, a dispersion, and an effervescent composition.
77. The method of any one of embodiments 52, wherein the lysostaphin variant or pharmaceutical composition is a parenteral formulation selected from the group consisting of intravenous, intraarterial, intraperitoneal, intrathecal, intradermal, epidural, intracerebroventricular, intraurethral, intrasternal, intracranial, intratumoral, intramuscular and subcutaneous.
78. The method of any one of embodiments 52, wherein the lysostaphin variant or pharmaceutical composition as an animal feed or animal feed supplement.
79. The method of any one of embodiments 52, wherein the lysostaphin variant or pharmaceutical composition is performed once daily, twice daily, or three times daily.
80. The method of any one of embodiments 52, wherein the lysostaphin variant or pharmaceutical composition comprises a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A.
81. The method of embodiment 80, wherein the lysostaphin variant comprises the amino acid mutations N27T, G29S, T126N, G143S, N157D, and S233A.
82. The method of embodiment 80, wherein said variant comprises a polypeptide having the amino acid sequence of any one of SEQ ID NOs:9 to 108.
83. A method of treating an animal, said method comprising the steps of administering the lysostaphin variant of any one of embodiments 1 to 32, a pharmaceutical composition of any of embodiments 33 to 51, wherein the pharmaceutical composition or the veterinary composition reduces the levels of Gram positive bacterium in said animal.
84. The method of embodiment 83, wherein the Gram positive bacterium is a Staphylococcus bacterium or a Streptococcus bacterium.
85. The method of embodiment 83, wherein said animal is selected from the group consisting of humans, ruminant animals, poultry, swine, canines, felines, Mustelidae family, and Leporidae family.
86. The method of any one of embodiments 83, wherein said animal is a canine and the treatment is an oral treatment.
87. The method of any one of embodiments 83, wherein said treatment is an oral treatment.
88. The method of any one of embodiments 83, wherein the treatment is for mastitis and the lysostaphin variant or pharmaceutical composition is formulated for topical administration or injection.
89. The method of any one of embodiments 83, wherein the lysostaphin variant or pharmaceutical composition is administered at an amount of between 0.001 to 1000 mg of the lysostaphin variant.
90. The method of any one of embodiments 83, wherein the lysostaphin variant or pharmaceutical composition is an oral formulation selected from the group consisting of a tablet, a capsule, a suspension, an emulsion, a syrup, a colloidal dispersion, a dispersion, and an effervescent composition.
91. The method of any one of embodiments 83, wherein the lysostaphin variant or pharmaceutical composition comprises a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A.
92. The method of embodiment 91, wherein the lysostaphin variant comprises the amino acid mutations N27T, G29S, T126N, G143S, N157D, and S233A.
93. The method of any one of embodiments 83 to 91, wherein said variant comprises a polypeptide having the amino acid sequence of any one of SEQ ID NOs:9 to 108.
94. A recombinant nucleic acid vector comprising a transcription promoter, a nucleic acid sequence encoding a lysostaphin polypeptide sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A, and a transcriptional terminator.
95. The recombinant nucleic acid vector of embodiment 94, wherein said variant comprises a polypeptide having the amino acid sequence of any one of SEQ ID NOs:9 to 108.
96. A method of producing a lysostaphin variant comprising a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A, said method comprising inducing expression of the lysostaphin polypeptide from the recombinant nucleic acid vector of embodiment 94 in a cell.
97. The method of embodiment 96, wherein said variant comprises a polypeptide having the amino acid sequence of any one of SEQ ID NOs:9 to 108.
98. The method of embodiment 96, wherein said cell is selected from a bacterium or yeast.
99. The method of embodiment 98, wherein said bacterium is E. coli or B. subtilis.
100. A method of treating colonization of medical devices comprising applying a lysostaphin polypeptide variant to the surface of a medical device.
101. The method of embodiment 100, wherein said surface is a plastic, glass, metal surface.
102. The method of embodiment 100, wherein said treatment is the reduction of surface colonization by S. aureus or S. epidermidis.
103. The method of embodiment 102, wherein said surface colonization comprises a biofilm.
104. A lysostaphin variant comprising a lysostaphin sequence and one or more amino acid mutations.
105. The lysostaphin variant of embodiment 104, any other suitable embodiment, or any combination of suitable embodiments, wherein the lysostaphin sequence comprises SEQ ID NO:1.
106. The lysostaphin variant of embodiment 104, any other suitable embodiment, or any combination of suitable embodiments, wherein the lysostaphin sequence consists essentially of SEQ ID NO:1.
107. The lysostaphin variant of embodiment 104, any other suitable embodiment, or any combination of suitable embodiments, wherein the lysostaphin sequence consists of SEQ ID NO:1.
108. The lysostaphin variant of embodiment 104, any other suitable embodiment, or any combination of suitable embodiments, wherein the lysostaphin sequence comprises a sequence having at least 60% homology to SEQ ID NO:1.
109. The lysostaphin variant of embodiment 104, any other suitable embodiment, or any combination of suitable embodiments, wherein the lysostaphin sequence comprises a sequence having at least 70% homology to SEQ ID NO:1.
110. The lysostaphin variant of embodiment 104, any other suitable embodiment, or any combination of suitable embodiments, wherein the lysostaphin sequence comprises a sequence having at least 80% homology to SEQ ID NO:1.
111. The lysostaphin variant of embodiment 104, any other suitable embodiment, or any combination of suitable embodiments, wherein the lysostaphin sequence comprises a sequence having at least 90% homology to SEQ ID NO:1.
112. The lysostaphin variant of embodiment 104, any other suitable embodiment, or any combination of suitable embodiments, wherein the lysostaphin sequence comprises a sequence having at least 95% homology to SEQ ID NO:1.
113. The lysostaphin variant of embodiment 104, any other suitable embodiment, or any combination of suitable embodiments, wherein the lysostaphin sequence comprises a sequence having at least 99% homology to SEQ ID NO:1.
114. The lysostaphin variant of embodiment 104, any other suitable embodiment, or any combination of suitable embodiments, wherein the lysostaphin sequence comprises SEQ ID NO:2.
115. The lysostaphin variant of embodiment 104, any other suitable embodiment, or any combination of suitable embodiments, wherein the lysostaphin sequence consists essentially of SEQ ID NO:2.
116. The lysostaphin variant of embodiment 104, any other suitable embodiment, or any combination of suitable embodiments, wherein the lysostaphin sequence consists of SEQ ID NO:2.
117. The lysostaphin variant of embodiment 104, any other suitable embodiment, or any combination of suitable embodiments, wherein the lysostaphin sequence comprises a sequence having at least 60% homology to SEQ ID NO:2.
118. The lysostaphin variant of embodiment 104, any other suitable embodiment, or any combination of suitable embodiments, wherein the lysostaphin sequence comprises a sequence having at least 70% homology to SEQ ID NO:2.
119. The lysostaphin variant of embodiment 104, any other suitable embodiment, or any combination of suitable embodiments, wherein the lysostaphin sequence comprises a sequence having at least 80% homology to SEQ ID NO:2.
120. The lysostaphin variant of embodiment 104, any other suitable embodiment, or any combination of suitable embodiments, wherein the lysostaphin sequence comprises a sequence having at least 90% homology to SEQ ID NO:2.
121. The lysostaphin variant of embodiment 104, any other suitable embodiment, or any combination of suitable embodiments, wherein the lysostaphin sequence comprises a sequence having at least 95% homology to SEQ ID NO:2.
122. The lysostaphin variant of embodiment 104, any other suitable embodiment, or any combination of suitable embodiments, wherein the lysostaphin sequence comprises a sequence having at least 99% homology to SEQ ID NO:2.
123. The lysostaphin variant of embodiment 104, any other suitable embodiment, or any combination of suitable embodiments, wherein the lysostaphin sequence comprises SEQ ID NO:3.
124. The lysostaphin variant of embodiment 104, any other suitable embodiment, or any combination of suitable embodiments, wherein the lysostaphin sequence consists essentially of SEQ ID NO:3.
125. The lysostaphin variant of embodiment 104, any other suitable embodiment, or any combination of suitable embodiments, wherein the lysostaphin sequence consists of SEQ ID NO:3.
126. The lysostaphin variant of embodiment 104, any other suitable embodiment, or any combination of suitable embodiments, wherein the lysostaphin sequence comprises a sequence having at least 60% homology to SEQ ID NO:3.
127. The lysostaphin variant of embodiment 104, any other suitable embodiment, or any combination of suitable embodiments, wherein the lysostaphin sequence comprises a sequence having at least 70% homology to SEQ ID NO:3.
128. The lysostaphin variant of embodiment 104, any other suitable embodiment, or any combination of suitable embodiments, wherein the lysostaphin sequence comprises a sequence having at least 80% homology to SEQ ID NO:3.
129. The lysostaphin variant of embodiment 104, any other suitable embodiment, or any combination of suitable embodiments, wherein the lysostaphin sequence comprises a sequence having at least 90% homology to SEQ ID NO:3.
130. The lysostaphin variant of embodiment 104, any other suitable embodiment, or any combination of suitable embodiments, wherein the lysostaphin sequence comprises a sequence having at least 95% homology to SEQ ID NO:3.
131. The lysostaphin variant of embodiment 104, any other suitable embodiment, or any combination of suitable embodiments, wherein the lysostaphin sequence comprises a sequence having at least 99% homology to SEQ ID NO:3.
132. The lysostaphin variant of embodiment 104, any other suitable embodiment, or any combination of suitable embodiments, wherein the one or more amino acid mutation is selected from the group consisting of the mutations in Table 3, and any combination thereof:
133. The lysostaphin variant of embodiment 104, any other suitable embodiment, or any combination of suitable embodiments, wherein the amino acid mutation is G25P.
134. The lysostaphin variant of embodiment 104, any other suitable embodiment, or any combination of suitable embodiments, wherein the amino acid mutation is G25S.
135. The lysostaphin variant of embodiment 104, any other suitable embodiment, or any combination of suitable embodiments, wherein the amino acid mutation is G25T.
136. The lysostaphin variant of embodiment 104, any other suitable embodiment, or any combination of suitable embodiments, wherein the amino acid mutation is N27E.
137. The lysostaphin variant of embodiment 104, any other suitable embodiment, or any combination of suitable embodiments, wherein the amino acid mutation is N27T.
138. The lysostaphin variant of embodiment 104, any other suitable embodiment, or any combination of suitable embodiments, wherein the amino acid mutation is N27D.
139. The lysostaphin variant of embodiment 104, any other suitable embodiment, or any combination of suitable embodiments, wherein the amino acid mutation is N27V.
140. The lysostaphin variant of embodiment 104, any other suitable embodiment, or any combination of suitable embodiments, wherein the amino acid mutation is N27S.
141. The lysostaphin variant of embodiment 104, any other suitable embodiment, or any combination of suitable embodiments, wherein the amino acid mutation is N27A.
142. The lysostaphin variant of embodiment 104, any other suitable embodiment, or any combination of suitable embodiments, wherein the amino acid mutation is G29S.
143. The lysostaphin variant of embodiment 104, any other suitable embodiment, or any combination of suitable embodiments, wherein the amino acid mutation is G29T.
144. The lysostaphin variant of embodiment 104, any other suitable embodiment, or any combination of suitable embodiments, wherein the amino acid mutation is G56S.
145. The lysostaphin variant of embodiment 104, any other suitable embodiment, or any combination of suitable embodiments, wherein the amino acid mutation is G62S.
146. The lysostaphin variant of embodiment 104, any other suitable embodiment, or any combination of suitable embodiments, wherein the amino acid mutation is T126N.
147. The lysostaphin variant of embodiment 104, any other suitable embodiment, or any combination of suitable embodiments, wherein the amino acid mutation is G143S.
148. The lysostaphin variant of embodiment 104, any other suitable embodiment, or any combination of suitable embodiments, wherein the amino acid mutation is G144Q.
149. The lysostaphin variant of embodiment 104, any other suitable embodiment, or any combination of suitable embodiments, wherein the amino acid mutation is N157D.
150. The lysostaphin variant of embodiment 104, any other suitable embodiment, or any combination of suitable embodiments, wherein the amino acid mutation is K158Q.
151. The lysostaphin variant of embodiment 104, any other suitable embodiment, or any combination of suitable embodiments, wherein the amino acid mutation is S233A.
152. The lysostaphin variant of embodiment 104, any other suitable embodiment, or any combination of suitable embodiments, wherein the amino acid mutation is N27T/G29S/T126N/G143S/N157D/S233A.
153. The lysostaphin variant of embodiment 104, any other suitable embodiment, or any combination of suitable embodiments, wherein the amino acid mutation is G25T/G29S/T126N/G143S/N157D/S233A.
154. The lysostaphin variant of embodiment 104, any other suitable embodiment, or any combination of suitable embodiments, wherein the amino acid mutation is G25T/N27S/G29S/T126N/G143S/N157D/S233A.
155. The lysostaphin variant of embodiment 104, any other suitable embodiment, or any combination of suitable embodiments, wherein the amino acid mutation is G25T/G29S/T126N/G143S/S233A.
156. The lysostaphin variant of embodiment 104, any other suitable embodiment, or any combination of suitable embodiments, wherein the amino acid mutation is G25T/G29S/G56S/T126N/G143S/S233A.
157. The lysostaphin variant of embodiment 104, any other suitable embodiment, or any combination of suitable embodiments, wherein the amino acid mutation is G25T/G28S/G56S/G62S/T126N/G143S/S233A.
158. The lysostaphin variant of embodiment 104, any other suitable embodiment, or any combination of suitable embodiments, wherein the lysostaphin variant is SEQ ID NO: 4.
159. The lysostaphin variant of embodiment 104, any other suitable embodiment, or any combination of suitable embodiments, wherein the lysostaphin variant comprises SEQ ID NO: 4.
160. The lysostaphin variant of embodiment 104, any other suitable embodiment, or any combination of suitable embodiments, wherein the lysostaphin variant consists essentially of SEQ ID NO: 4.
161. The lysostaphin variant of embodiment 104, any other suitable embodiment, or any combination of suitable embodiments, wherein the lysostaphin variant consists of SEQ ID NO: 4.
162. The lysostaphin variant of embodiment 104, any other suitable embodiment, or any combination of suitable embodiments, wherein the lysostaphin variant is has a lower amount of degradation compared to wild type lysostaphin.
163. The lysostaphin variant of embodiment 162, any other suitable embodiment, or any combination of suitable embodiments, wherein the degradation is self-degradation.
164. The lysostaphin variant of embodiment 162, any other suitable embodiment, or any combination of suitable embodiments, wherein the degradation occurs during fermentation of the lysostaphin or the lysostaphin variant.
165. The lysostaphin variant of embodiment 162, any other suitable embodiment, or any combination of suitable embodiments, wherein the degradation occurs at room temperature.
166. The lysostaphin variant of embodiment 162, any other suitable embodiment, or any combination of suitable embodiments, wherein the degradation occurs at 20° C.
167. The lysostaphin variant of embodiment 162, any other suitable embodiment, or any combination of suitable embodiments, wherein the degradation occurs at 37° C.
168. The lysostaphin variant of embodiment 162, any other suitable embodiment, or any combination of suitable embodiments, wherein the degradation occurs between 20° C. and 37° C.
169. A pharmaceutical composition comprising the lysostaphin variant of any of embodiments 104-168.
170. The pharmaceutical composition of embodiment 168, any other suitable embodiment, or any combination of suitable embodiments, wherein the pharmaceutical composition comprises one or more carriers.
171. The pharmaceutical composition of embodiment 170, any other suitable embodiment, or any combination of suitable embodiments, wherein the carrier is selected from the group consisting of saline, glucose, alcohols, glycols, esters, amides, and a combination thereof.
172. The pharmaceutical composition of embodiment 170, any other suitable embodiment, or any combination of suitable embodiments, wherein the carrier comprises one or more salts.
173. The pharmaceutical composition of embodiment 170, any other suitable embodiment, or any combination of suitable embodiments, wherein the carrier does not comprise salt.
174. The pharmaceutical composition of embodiment 170, any other suitable embodiment, or any combination of suitable embodiments, wherein the carrier comprises water.
175. The pharmaceutical composition of embodiment 170, any other suitable embodiment, or any combination of suitable embodiments, wherein the carrier is water.
176. The pharmaceutical composition of embodiment 170, any other suitable embodiment, or any combination of suitable embodiments, wherein the carrier comprises liquid.
177. The pharmaceutical composition of embodiment 170, any other suitable embodiment, or any combination of suitable embodiments, wherein the carrier is a liquid.
178. The pharmaceutical composition of embodiment 170, any other suitable embodiment, or any combination of suitable embodiments, wherein the carrier comprises a solution.
179. The pharmaceutical composition of embodiment 170, any other suitable embodiment, or any combination of suitable embodiments, wherein the carrier is a solution.
180. The pharmaceutical composition of embodiment 170, any other suitable embodiment, or any combination of suitable embodiments, wherein the carrier comprises saline.
181. The pharmaceutical composition of embodiment 170, any other suitable embodiment, or any combination of suitable embodiments, wherein the carrier is saline.
182. The pharmaceutical composition of embodiment 170, any other suitable embodiment, or any combination of suitable embodiments, wherein the carrier comprises a soft gel capsule.
183. The pharmaceutical composition of embodiment 170, any other suitable embodiment, or any combination of suitable embodiments, wherein the carrier is a soft gel capsule.
184. The pharmaceutical composition of embodiment 170, any other suitable embodiment, or any combination of suitable embodiments, wherein the carrier comprises powder.
185. The pharmaceutical composition of embodiment 170, any other suitable embodiment, or any combination of suitable embodiments, wherein the carrier is a powder.
186. The pharmaceutical composition of embodiment 185, any other suitable embodiment, or any combination of suitable embodiments, wherein the powder is contained in a capsule.
187. The pharmaceutical composition of embodiment 185, any other suitable embodiment, or any combination of suitable embodiments, wherein the powder is contained in a gel cap.
188. The pharmaceutical composition of embodiment 170, any other suitable embodiment, or any combination of suitable embodiments, wherein the carrier comprises a solid.
189. The pharmaceutical composition of embodiment 170, any other suitable embodiment, or any combination of suitable embodiments, wherein the carrier is a solid.
190. The pharmaceutical composition of embodiment 189, any other suitable embodiment, or any combination of suitable embodiments, wherein the solid is a chewable tablet.
191. The pharmaceutical composition of embodiment 190, any other suitable embodiment, or any combination of suitable embodiments, wherein the chewable tablet is flavored.
192. The pharmaceutical composition of embodiment 190, any other suitable embodiment, or any combination of suitable embodiments, wherein the chewable tablet is not flavored.
193. The pharmaceutical composition of embodiment 189, any other suitable embodiment, or any combination of suitable embodiments, wherein the solid is a chewy treat.
194. The pharmaceutical composition of embodiment 170, any other suitable embodiment, or any combination of suitable embodiments, wherein the carrier comprises a mixture of a solid and a liquid.
195. The pharmaceutical composition of embodiment 170, any other suitable embodiment, or any combination of suitable embodiments, wherein the carrier is a mixture of a solid and a liquid.
196. The pharmaceutical composition of embodiment 170, any other suitable embodiment, or any combination of suitable embodiments, wherein the carrier comprises gas.
197. The pharmaceutical composition of embodiment 170, any other suitable embodiment, or any combination of suitable embodiments, wherein the carrier is a gas.
198. The pharmaceutical composition of embodiment 170, any other suitable embodiment, or any combination of suitable embodiments, wherein the carrier comprises a dispersion.
199. The pharmaceutical composition of embodiment 170, any other suitable embodiment, or any combination of suitable embodiments, wherein the carrier is a dispersion.
200. The pharmaceutical composition of embodiment 170, any other suitable embodiment, or any combination of suitable embodiments, wherein the carrier comprises a suspension.
201. The pharmaceutical composition of embodiment 170, any other suitable embodiment, or any combination of suitable embodiments, wherein the carrier is a suspension.
202. The pharmaceutical composition of embodiment 170, any other suitable embodiment, or any combination of suitable embodiments, wherein the carrier comprises a solution suspension.
203. The pharmaceutical composition of embodiment 170, any other suitable embodiment, or any combination of suitable embodiments, wherein the carrier is a solution suspension.
204. The pharmaceutical composition of embodiment 170, any other suitable embodiment, or any combination of suitable embodiments, wherein the carrier comprises a crystalline suspension.
205. The pharmaceutical composition of embodiment 170, any other suitable embodiment, or any combination of suitable embodiments, wherein the carrier is a crystalline suspension.
206. The pharmaceutical composition of embodiment 170, any other suitable embodiment, or any combination of suitable embodiments, wherein the carrier comprises a slurry.
207. The pharmaceutical composition of embodiment 170, any other suitable embodiment, or any combination of suitable embodiments, wherein the carrier is a slurry.
208. The pharmaceutical composition of embodiment 170, any other suitable embodiment, or any combination of suitable embodiments, wherein the carrier comprises a spray.
209. The pharmaceutical composition of embodiment 170, any other suitable embodiment, or any combination of suitable embodiments, wherein the carrier is a spray.
210. The pharmaceutical composition of embodiment 170, any other suitable embodiment, or any combination of suitable embodiments, wherein the carrier is wet.
211. The pharmaceutical composition of embodiment 170, any other suitable embodiment, or any combination of suitable embodiments, wherein the carrier is dry.
212. The pharmaceutical composition of embodiment 170, any other suitable embodiment, or any combination of suitable embodiments, wherein the carrier comprises a surfactant.
213. The pharmaceutical composition of embodiment 168, any other suitable embodiment, or any combination of suitable embodiments, wherein the pharmaceutical composition comprises an encapsulation composition for the lysostaphin variant.
214. The pharmaceutical composition of embodiment 168, any other suitable embodiment, or any combination of suitable embodiments, wherein the pharmaceutical composition comprises a further active pharmaceutical ingredient (API).
215. The pharmaceutical composition of embodiment 168, any other suitable embodiment, or any combination of suitable embodiments, wherein the pharmaceutical composition comprises a second therapeutic agent.
216. The pharmaceutical composition of embodiment 168, any other suitable embodiment, or any combination of suitable embodiments, wherein the pharmaceutical composition is lyophilized.
217. The pharmaceutical composition of embodiment 168, any other suitable embodiment, or any combination of suitable embodiments, wherein the lysostaphin variant is encapsulated.
218. The pharmaceutical composition of embodiment 168, any other suitable embodiment, or any combination of suitable embodiments, wherein the lysostaphin variant is encapsulated in one or more polymer compositions.
219. A veterinary composition comprising the lysostaphin variant of any one of embodiments 104-168.
220. The veterinary composition of embodiment 219, any other suitable embodiment, or any combination of suitable embodiments, wherein the veterinary composition comprises one or more carriers.
221. The veterinary composition of embodiment 219, any other suitable embodiment, or any combination of suitable embodiments, wherein the carrier is selected from the group consisting of saline, glucose, alcohols, glycols, esters, amides, and a combination thereof.
222. The veterinary composition of embodiment 219, any other suitable embodiment, or any combination of suitable embodiments, wherein the carrier comprises one or more salts.
223. The veterinary composition of embodiment 219, any other suitable embodiment, or any combination of suitable embodiments, wherein the carrier does not comprise salt.
224. The veterinary composition of embodiment 219, any other suitable embodiment, or any combination of suitable embodiments, wherein the carrier comprises water.
225. The veterinary composition of embodiment 219, any other suitable embodiment, or any combination of suitable embodiments, wherein the carrier is water.
226. The veterinary composition of embodiment 219, any other suitable embodiment, or any combination of suitable embodiments, wherein the carrier comprises liquid.
227. The veterinary composition of embodiment 219, any other suitable embodiment, or any combination of suitable embodiments, wherein the carrier is a liquid.
228. The veterinary composition of embodiment 219, any other suitable embodiment, or any combination of suitable embodiments, wherein the carrier comprises a solution.
229. The veterinary composition of embodiment 219, any other suitable embodiment, or any combination of suitable embodiments, wherein the carrier is a solution.
230. The veterinary composition of embodiment 219, any other suitable embodiment, or any combination of suitable embodiments, wherein the carrier comprises saline.
231. The veterinary composition of embodiment 219, any other suitable embodiment, or any combination of suitable embodiments, wherein the carrier is saline.
232. The veterinary composition of embodiment 219, any other suitable embodiment, or any combination of suitable embodiments, wherein the carrier comprises a soft gel capsule.
233. The veterinary composition of embodiment 219, any other suitable embodiment, or any combination of suitable embodiments, wherein the carrier is a soft gel capsule.
234. The veterinary composition of embodiment 219, any other suitable embodiment, or any combination of suitable embodiments, wherein the carrier comprises a powder.
235. The veterinary composition of embodiment 219, any other suitable embodiment, or any combination of suitable embodiments, wherein the carrier is a powder.
236. The veterinary composition of embodiment 219, any other suitable embodiment, or any combination of suitable embodiments, wherein the powder is contained in a capsule.
237. The veterinary composition of embodiment 219, any other suitable embodiment, or any combination of suitable embodiments, wherein the powder is contained in a gel cap.
238. The veterinary composition of embodiment 219, any other suitable embodiment, or any combination of suitable embodiments, wherein the carrier comprises a solid.
239. The veterinary composition of embodiment 219, any other suitable embodiment, or any combination of suitable embodiments, wherein the carrier is a solid.
240. The veterinary composition of embodiment 239, any other suitable embodiment, or any combination of suitable embodiments, wherein the solid is a chewable tablet.
241. The veterinary composition of embodiment 240, any other suitable embodiment, or any combination of suitable embodiments, wherein the chewable tablet is flavored.
242. The veterinary composition of embodiment 240, any other suitable embodiment, or any combination of suitable embodiments, wherein the chewable tablet is not flavored.
243. The veterinary composition of embodiment 239, any other suitable embodiment, or any combination of suitable embodiments, wherein the solid is a chewy treat.
244. The veterinary composition of embodiment 219, any other suitable embodiment, or any combination of suitable embodiments, wherein the carrier comprises a mixture of a solid and a liquid.
245. The veterinary composition of embodiment 219, any other suitable embodiment, or any combination of suitable embodiments, wherein the carrier is a mixture of a solid and a liquid.
246. The veterinary composition of embodiment 219, any other suitable embodiment, or any combination of suitable embodiments, wherein the carrier comprises gas.
247. The veterinary composition of embodiment 219, any other suitable embodiment, or any combination of suitable embodiments, wherein the carrier is a gas.
248. The veterinary composition of embodiment 219, any other suitable embodiment, or any combination of suitable embodiments, wherein the carrier comprises a dispersion.
249. The veterinary composition of embodiment 219, any other suitable embodiment, or any combination of suitable embodiments, wherein the carrier is a dispersion.
250. The veterinary composition of embodiment 219, any other suitable embodiment, or any combination of suitable embodiments, wherein the carrier comprises a suspension.
251. The veterinary composition of embodiment 219, any other suitable embodiment, or any combination of suitable embodiments, wherein the carrier is a suspension.
252. The veterinary composition of embodiment 219, any other suitable embodiment, or any combination of suitable embodiments, wherein the carrier comprises a solution suspension.
253. The veterinary composition of embodiment 219, any other suitable embodiment, or any combination of suitable embodiments, wherein the carrier is a solution suspension.
254. The veterinary composition of embodiment 219, any other suitable embodiment, or any combination of suitable embodiments, wherein the carrier comprises a crystalline suspension.
255. The veterinary composition of embodiment 219, any other suitable embodiment, or any combination of suitable embodiments, wherein the carrier is a crystalline suspension.
256. The veterinary composition of embodiment 219, any other suitable embodiment, or any combination of suitable embodiments, wherein the carrier comprises a slurry.
257. The veterinary composition of embodiment 219, any other suitable embodiment, or any combination of suitable embodiments, wherein the carrier is a slurry.
258. The veterinary composition of embodiment 219, any other suitable embodiment, or any combination of suitable embodiments, wherein the carrier comprises a spray.
259. The veterinary composition of embodiment 219, any other suitable embodiment, or any combination of suitable embodiments, wherein the carrier is a spray.
260. The veterinary composition of embodiment 219, any other suitable embodiment, or any combination of suitable embodiments, wherein the carrier is wet.
261. The veterinary composition of embodiment 219, any other suitable embodiment, or any combination of suitable embodiments, wherein the carrier is dry.
262. The veterinary composition of embodiment 219, any other suitable embodiment, or any combination of suitable embodiments, wherein the carrier comprises a surfactant.
263. The veterinary composition of embodiment 219, any other suitable embodiment, or any combination of suitable embodiments, wherein the veterinary composition comprises an encapsulation composition for the lysostaphin variant.
264. The veterinary composition of embodiment 219, any other suitable embodiment, or any combination of suitable embodiments, wherein the veterinary composition comprises a further active pharmaceutical ingredient (API).
265. The veterinary composition of embodiment 219, any other suitable embodiment, or any combination of suitable embodiments, wherein the veterinary composition comprises a second therapeutic agent.
266. The veterinary composition of embodiment 219, any other suitable embodiment, or any combination of suitable embodiments, wherein the veterinary composition is lyophilized.
267. The veterinary composition of embodiment 219, any other suitable embodiment, or any combination of suitable embodiments, wherein the lysostaphin variant is encapsulated.
268. The veterinary composition of embodiment 219, any other suitable embodiment, or any combination of suitable embodiments, wherein the lysostaphin variant is encapsulated in one or more polymer compositions.
269. A method of treating an infection in an animal, said method comprising the step of administering the pharmaceutical composition of any of embodiments 169-218 or the veterinary composition of any of embodiments 219-268 to an animal, wherein the pharmaceutical composition or the veterinary composition reduces one or more symptoms associated with the infection in the animal.
270. The method of embodiment 269, any other suitable embodiment, or any combination of suitable embodiments, wherein the infection is caused by a Gram positive bacterium.
271. The method of embodiment 269, any other suitable embodiment, or any combination of suitable embodiments, wherein the infection is caused by a Staphylococcus bacterium.
272. The method of embodiment 271, any other suitable embodiment, or any combination of suitable embodiments, wherein the Staphylococcus bacterium is Staphylococcus aureus.
273. The method of embodiment 271, any other suitable embodiment, or any combination of suitable embodiments, wherein the Staphylococcus bacterium is Staphylococcus pseudintermedius.
274. The method of embodiment 271, any other suitable embodiment, or any combination of suitable embodiments, wherein the Staphylococcus bacterium is Staphylococcus agnetis.
275. The method of embodiment 269, any other suitable embodiment, or any combination of suitable embodiments, wherein the infection is caused by a Streptococcus bacterium.
276. The method of embodiment 275, any other suitable embodiment, or any combination of suitable embodiments, wherein the Streptococcus bacterium is Streptococcus suis.
277. The method of embodiment 269, any other suitable embodiment, or any combination of suitable embodiments, wherein the infection is a Methicillin Resistant Staphylococcus aureus (MRSA) infection.
278. The method of embodiment 269, any other suitable embodiment, or any combination of suitable embodiments, wherein the infection is a Vancomycin Intermediate Staphylococcus aureus (VISA) infection.
279. The method of embodiment 269, any other suitable embodiment, or any combination of suitable embodiments, wherein the infection is a Vancomycin Resistant Staphylococcus aureus (VRSA) infection.
280. The method of embodiment 269, any other suitable embodiment, or any combination of suitable embodiments, wherein the animal is a human.
281. The method of embodiment 269, any other suitable embodiment, or any combination of suitable embodiments, wherein the animal is a ruminant.
282. The method of embodiment 281, any other suitable embodiment, or any combination of suitable embodiments, wherein the ruminant is selected from the group consisting of cattle, goats, sheep, giraffes, American Bison, European bison, yaks, water buffalo, deer, camels, alpacas, llamas, wildebeest, antelope, pronghorn, and nilgai.
283. The method of embodiment 281, any other suitable embodiment, or any combination of suitable embodiments, wherein the ruminant animal is a zoo animal.
284. The method of embodiment 281, any other suitable embodiment, or any combination of suitable embodiments, wherein the ruminant animal is a domestic animal.
285. The method of embodiment 281, any other suitable embodiment, or any combination of suitable embodiments, wherein the ruminant animal is a wild animal.
286. The method of embodiment 281, any other suitable embodiment, or any combination of suitable embodiments.
287. The method of embodiment 281, any other suitable embodiment, or any combination of suitable embodiments, wherein the ruminant animal is a livestock animal.
288. The method of embodiment 281, any other suitable embodiment, or any combination of suitable embodiments, wherein the ruminant animal is a bovine animal.
289. The method of embodiment 288, any other suitable embodiment, or any combination of suitable embodiments, wherein the bovine animal is a cattle.
290. The method of embodiment 288, any other suitable embodiment, or any combination of suitable embodiments, wherein the cattle is a meat-producing cattle.
291. The method of embodiment 288, any other suitable embodiment, or any combination of suitable embodiments, wherein the cattle is a milk-producing cattle.
292. The method of embodiment 288, any other suitable embodiment, or any combination of suitable embodiments, wherein the milk-producing cattle is a dairy cattle.
293. The method of embodiment 269, any other suitable embodiment, or any combination of suitable embodiments, wherein the animal is a poultry.
294. The method of embodiment 293, any other suitable embodiment, or any combination of suitable embodiments, wherein the poultry is selected from the group of ducks, geese, chickens, chickens, laying hens, broilers, turkeys, quails, ostriches, and turkeys.
295. The method of embodiment 269, any other suitable embodiment, or any combination of suitable embodiments, wherein the animal is a swine.
296. The method of embodiment 295, any other suitable embodiment, or any combination of suitable embodiments, wherein the swine is selected from the group consisting of pigs, boars, sows, gilts, and piglets.
297. The method of embodiment 269, any other suitable embodiment, or any combination of suitable embodiments, wherein the animal is a companion animal.
298. The method of embodiment 297, any other suitable embodiment, or any combination of suitable embodiments, wherein the companion animal is selected from the group consisting of canine, feline, rabbit, and ferret.
299. The method of embodiment 269, any other suitable embodiment, or any combination of suitable embodiments, wherein the pharmaceutical composition or the veterinary composition is administered at an amount of between 0.001 to 1000 mg of the lysostaphin variant.
300. The method of embodiment 269, any other suitable embodiment, or any combination of suitable embodiments, wherein the pharmaceutical composition or the veterinary composition is administered at a dose of about 0.001 to about 1000 mg of the lysostaphin variant per kg of animal body weight.
301. The method of embodiment 269, any other suitable embodiment, or any combination of suitable embodiments, wherein the pharmaceutical composition or the veterinary composition is an oral formulation.
302. The method of embodiment 301, any other suitable embodiment, or any combination of suitable embodiments, wherein the oral formulation is selected from the group consisting of a tablet, a capsule, a suspension, an emulsion, a syrup, a colloidal dispersion, a dispersion, and an effervescent composition.
303. The method of embodiment 301, any other suitable embodiment, or any combination of suitable embodiments, wherein the oral formulation is a suspension.
304. The method of embodiment 301, any other suitable embodiment, or any combination of suitable embodiments, wherein the oral formulation is a reconstitutable suspension.
305. The method of embodiment 269, any other suitable embodiment, or any combination of suitable embodiments, wherein the pharmaceutical composition or the veterinary composition is a parenteral formulation.
306. The method of embodiment 305, any other suitable embodiment, or any combination of suitable embodiments, wherein the parenteral formulation is selected from the group consisting of intravenous, intraarterial, intraperitoneal, intrathecal, intradermal, epidural, intracerebroventricular, intraurethral, intra-sternal, intracranial, intra-tumoral, intramuscular and subcutaneous.
307. The method of embodiment 269, any other suitable embodiment, or any combination of suitable embodiments, wherein the veterinary composition is administered as an animal feed.
308. The method of embodiment 269, any other suitable embodiment, or any combination of suitable embodiments, wherein the veterinary composition is administered as an animal feed supplement.
309. The method of embodiment 269, any other suitable embodiment, or any combination of suitable embodiments, wherein the step of administering the veterinary formulation to the animal is performed in conjunction with feeding the animal.
310. The method of embodiment 269, any other suitable embodiment, or any combination of suitable embodiments, wherein the step of administering the veterinary formulation to the animal is performed while feeding the animal.
311. The method of embodiment 269, any other suitable embodiment, or any combination of suitable embodiments, wherein the step of administering the veterinary formulation to the animal is performed concurrently with feeding the animal.
312. The method of embodiment 269, any other suitable embodiment, or any combination of suitable embodiments, wherein the step of administering the veterinary formulation to the animal is performed simultaneously with feeding the animal.
313. The method of embodiment 269, any other suitable embodiment, or any combination of suitable embodiments, wherein the step of administering the veterinary formulation to the animal is performed independently of feeding the animal.
314. The method of embodiment 269, any other suitable embodiment, or any combination of suitable embodiments, wherein the step of administering the veterinary formulation to the animal is performed after feeding the animal.
315. The method of embodiment 269, any other suitable embodiment, or any combination of suitable embodiments, wherein the step of administering the veterinary formulation to the animal is performed daily.
316. The method of embodiment 269, any other suitable embodiment, or any combination of suitable embodiments, wherein the step of administering the veterinary formulation to the animal is performed once per day.
317. The method of embodiment 269, any other suitable embodiment, or any combination of suitable embodiments, wherein the step of administering the veterinary formulation to the animal is performed twice per day.
318. The method of embodiment 269, any other suitable embodiment, or any combination of suitable embodiments, wherein the step of administering the veterinary formulation to the animal is performed up to three times per day.

### EXAMPLES

### Example 1: Degradation of Wild Type Lysostaphin

The instant example describes the degradation observed during fermentation of wild type lysostaphin. For instance, self-degradation of lysostaphin was observed after incubation at room temperature or at 37° C.

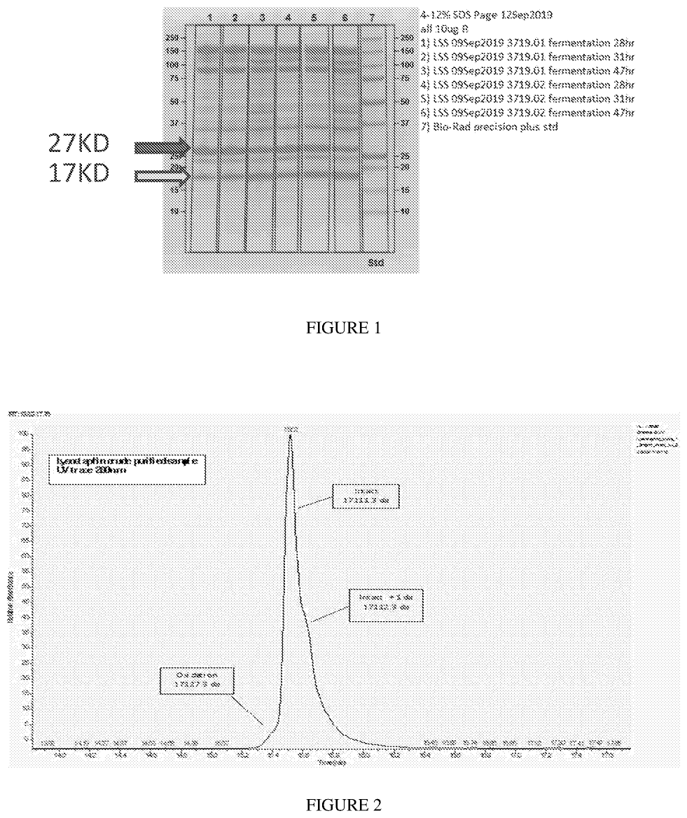

FIG. 1 identifies various bands of lysostaphin following fermentation at various time points. Two bands (i.e., a band at 27 kD and a band at 17 kD) were analyzed for the presence of lysostaphin at fermentation time points of 28, 31, 47, 50, and 53 hours and the results presented in Table 4:

TABLE 4
Densitometry Analysis of Lysostaphin at various fermentation times

| | | 27K band | 27K band | 17K band | 17K band |
|---|---|---|---|---|---|
| Tank | Time | volume | Relative % | volume | Relative % |
| 1 | 28 | 1,291,956 | 62.0 | 792,051 | 38.0 |
| 1 | 31 | 1,282,890 | 57.3 | 956,760 | 42.7 |
| 1 | 47 | 803,855 | 42.6 | 1,083,745 | 57.4 |
| 1 | 50 | 704,832 | 39.6 | 1,077,120 | 60.4 |
| 1 | 53 | 631,176 | 40.1 | 941,876 | 59.9 |
| 2 | 28 | 1,075,590 | 55.3 | 869,421 | 44.7 |
| 2 | 31 | 1,148,091 | 49.7 | 1,162,788 | 50.3 |
| 2 | 47 | 671,391 | 35.9 | 1,197,819 | 64.1 |
| 2 | 50 | 552,552 | 34.4 | 1,052,220 | 65.6 |
| 2 | 53 | 570,128 | 34.5 | 1,080,040 | 65.5 |

The sequence that was identified by mass spec in the lower band was

(SEQ ID NO: 4)
AATHEHSAQWLNNYKKGYGYGPYPLGINGGMHYGVDFFMNIGTPVKAIS
SGKIVEAGWSNYGGGNQIGLIENDGVHRQWYMHLSKYNVKVGDYVKAGQ
IIGWSGSTGYSTAPHLHFQRMVNSFSNSTAQDPMPFLKSAGYGKAGGTV
TPTPNTGWKTN.

FIG. 2 shows intact mass analysis of the 17 kDa fragment. The intact mass matched the expected analysis based on the N-terminal and C-terminal cleavage sites determined from the in-gel digest evaluation. Further, some oxidation and a deamidation (+1 Da) were found, as well as a very small amount of the C-terminal fragment that was missing (the amount was so small that it was not detectable in the UV signal and was only present in the MS signal).

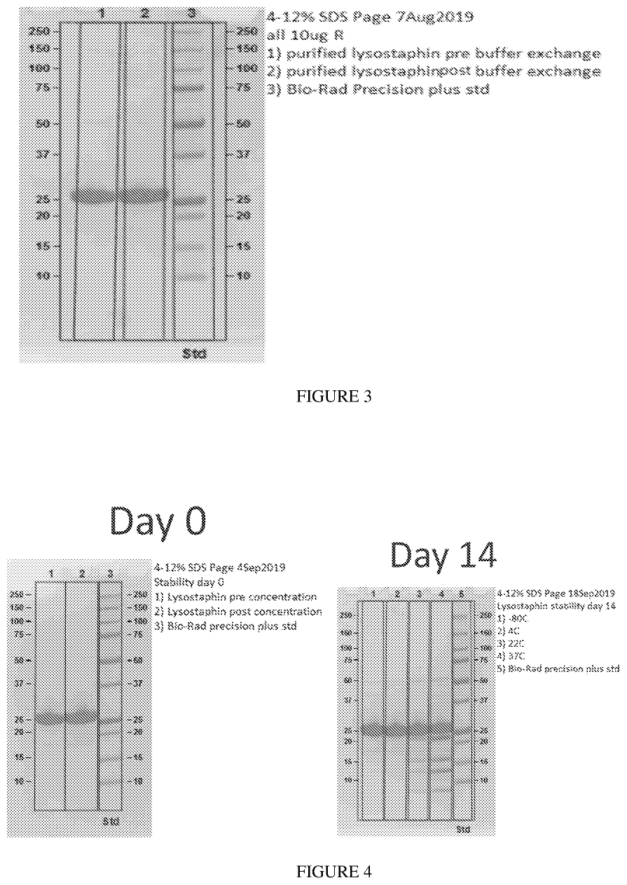

Furthermore, self-degradation of lysostaphin was observed after incubation at room temperature or at 37° C. As shown in FIG. 3, lysostaphin was expressed in P. lentus and purified in 50 mM Tris-HCl, 0.15 M NaCl pH 7.5 (Volume 700 mL; Concentration by BCA 2.9 mg/mL; Total Protein 2.03 g; Endotoxin <0.05 EU/mL).

As shown in FIG. 4, the purification was aliquoted into 200 uL/vial 10 vials and placed in each of the following temperatures: −80° C., 4° C., RT, 37° C. Then, 1 vial from each temperature was removed each workday for 2 weeks, an SDS PAGE gel was run and densitometry was performed then the vials were frozen at −80° C. for assaying.

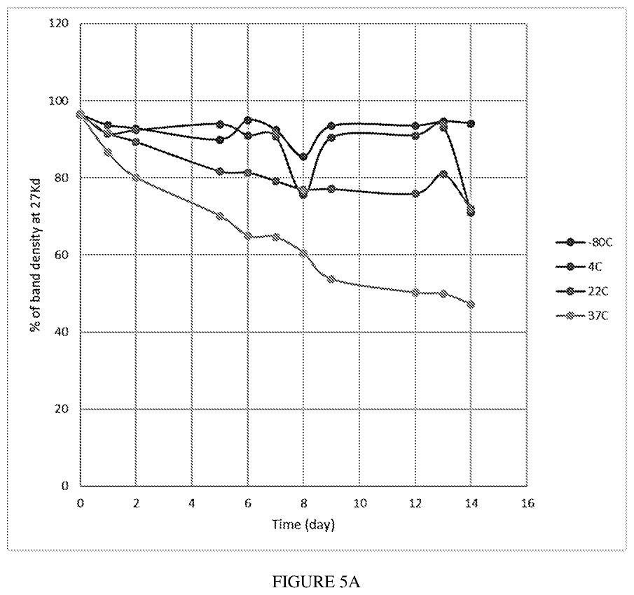

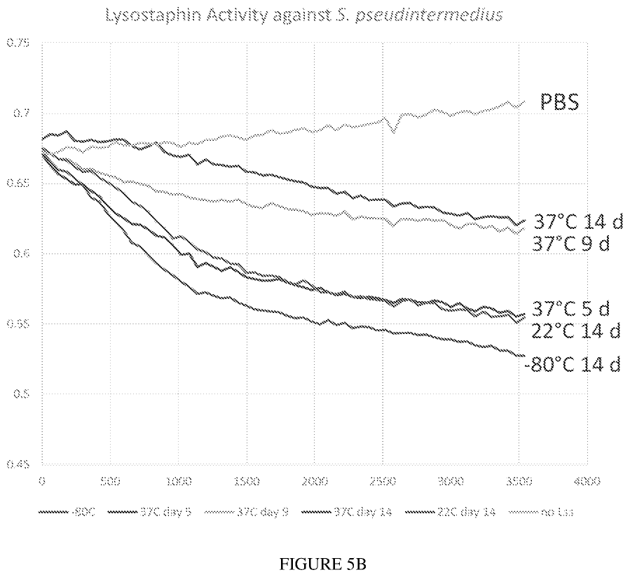

Protein integrity and lytic activity of lysostaphin against S. pseudintermedius are shown in FIGS. 5A-5B.

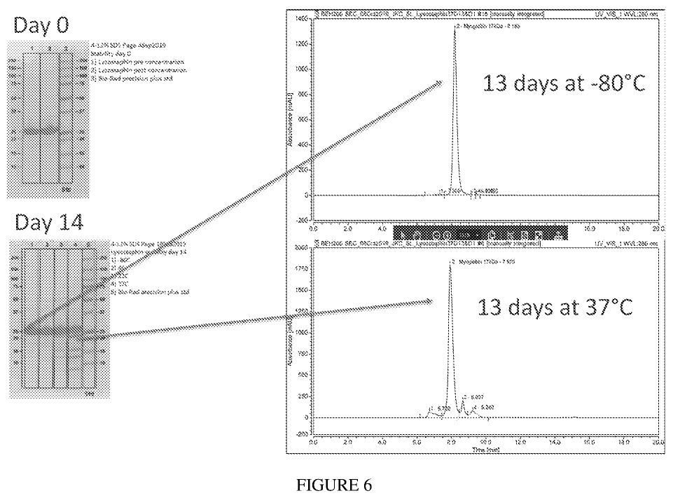

Furthermore, the SEC-HPLC of incubated lysostaphin samples is shown in FIG. 6.

According to the analysis in the instant example, the 17 kD band of lysostaphin observed in fermenters is not formed at any of the temperatures tested. Therefore, it is unlikely a self-cleavage event. Moreover, the incubation at 37° C. causes a clear decrease in lysostaphin activity, most likely due to deamination at N27 in addition to degradation.

### Example 2: Mass Spectrometry Analysis of Wild Type Lysostaphin Degradation

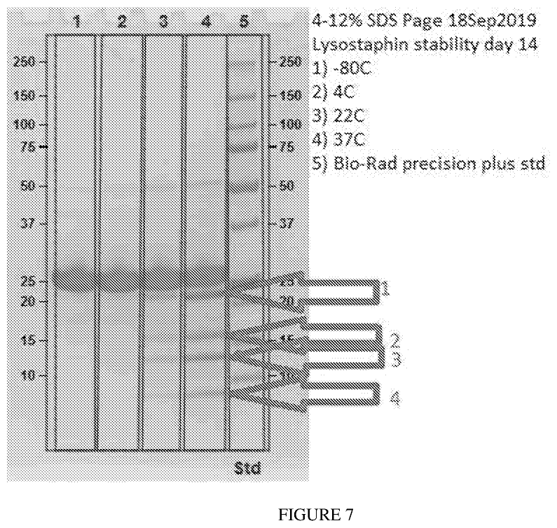

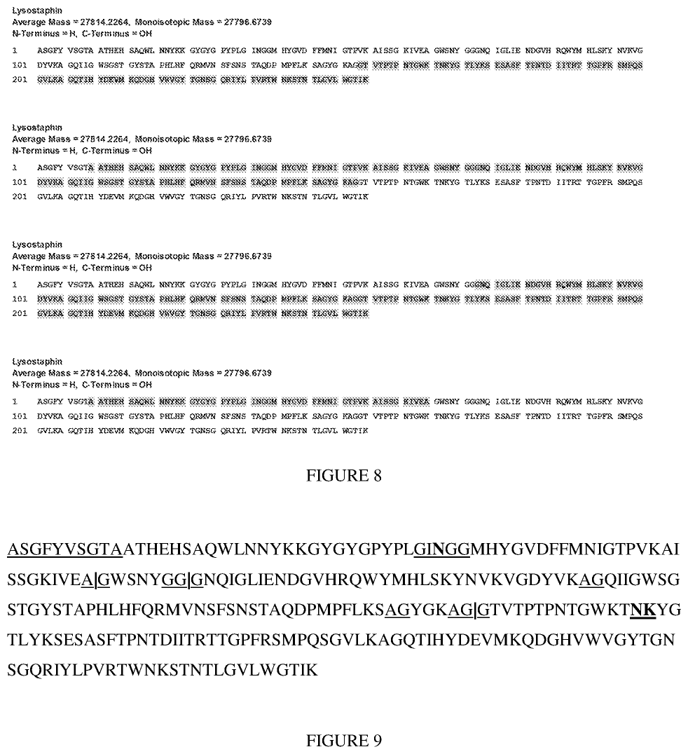

In the instant example, four bands of lower molecular weights from the SDS-PAGE lysostaphin analysis (see FIG. 7) were excised and subjected to further mass spec analysis. LC-ESIMS results were analyzed. Analysis of the band is shown in FIG. 8.

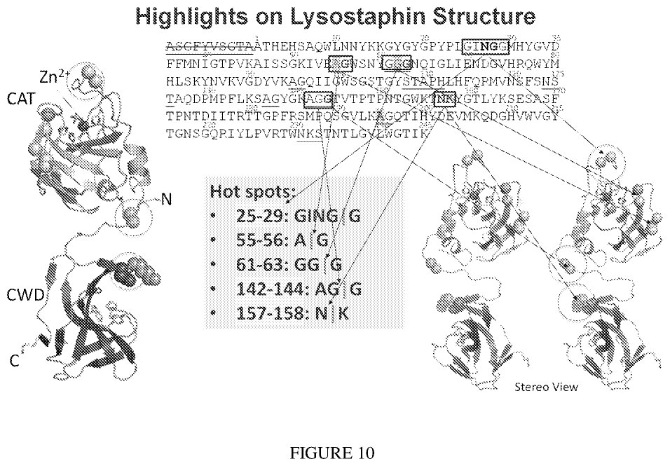

Following mass spec analysis, major cleavage points of lysostaphin (14 days at 37° C.) were identified. A summary of the analysis is found at FIG. 9 and i) identifies major cleavage points (shown with “I”), ii) demonstrated a missing sequence (ASGFYVSGT), iii) identified a deamidation site at N27, and iv) identified a cleavage site in fermenters at N157-K158. Furthermore, FIG. 10 shows various highlights on the lysostaphin structure.

According to the analysis in the instant example, two separate degradation events were identified. In fermenters, a major fragment (˜17 kD) is formed at the catalytic domain ending at N157. Purified lysostaphin at 15 mg/ml undergoes a self-degradation process at RT or 37° C. 3-4 Sites (at G|G, or A|G) identified by MS.

Thus, small residues as Gly or Ala at shorter lengths can be targets for its own proteolytic activity at lower rates. It was determined to generate lysostaphin variants to provide for a more stable version of lysostaphin.

### Example 3: Generation and Analysis of Lysostaphin Variants

The instant example concerns generation of lysostaphin variants using point designed to eliminate possible self-cleavage sites. Copies of the genes were ordered and proteins purified at GenScript. Lytic activity was tested against Staph pseudintermedius.

Table 5 shows various point mutations of the lysostaphin sequence and the rationale for such mutations.

TABLE 5
Lysostaphin Point mutations and Rationale

| Sequence | Reasons |
|---|---|
| ²⁵GINGG²⁹ | Cleavage, Deamination |
| (SEQ ID | |
| NO: 109) | |
| ¹²⁴N S T¹²⁶ | Glycosylation |
| ¹⁴³G G¹⁴⁴ | Cleavage |
| ¹⁵⁷N K¹⁵⁸ | Cleavage |
| ²³¹N K S²³³ | Glycosylation |

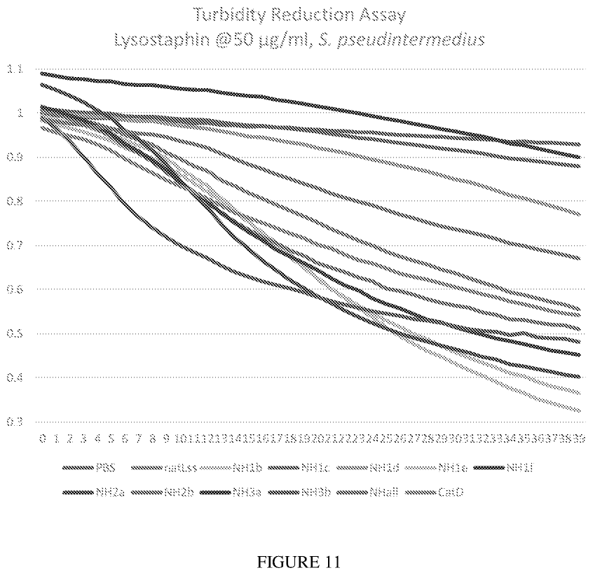

FIG. 11 shows a turbidity reduction assay of lysostaphin at 50 μg/ml in S. pseudintermedius.

TABLE 6
Lysostaphin variants and activity ranks

| Short | | Activity |
|---|---|---|
| Name | Variant | Rank |
| PBS | | |
| natLss | | 8 |
| NH1b | G25T | 1 |
| NH1c | N27E | 11 |
| NH1d | N27T | 10 |
| NH1e | G29S | 2 |
| NH1f | N27D | 12 |
| NH2a | G143S | 3 |
| NH2b | G144Q | 6 |
| NH3a | K158Q | 4 |
| NH3b | N157D | 5 |
| NHall | N27T/G29S/ | 9 |
| | T126N/G143S/ | |
| | N157D/S233A | |
| CatD | 1▭142 | 7 |

The identification of lysostaphin variants and their associated activity ranks are shown in Table 6.

Table 7 shows the identification of lysostaphin variants and their associated activity ranks in a turbidity assay in S. pseudintermedius. The in silico stability determination is also shown in Table 7.

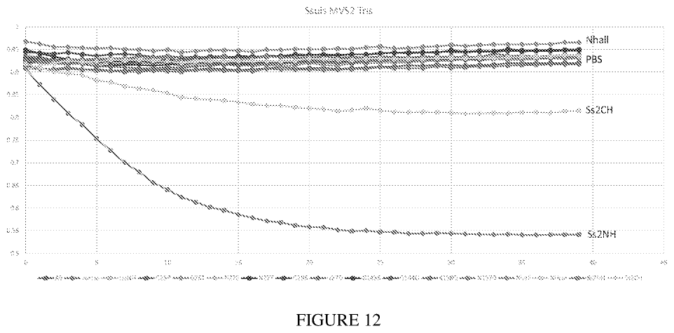

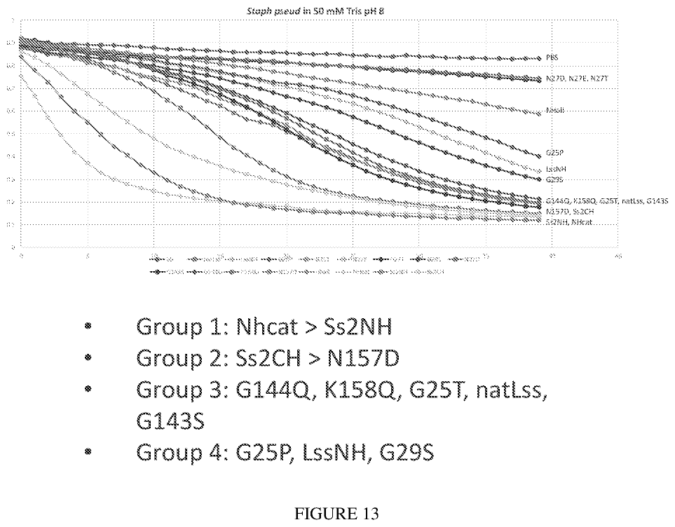

FIG. 12 shows a turbidity assay in S. suis (Tris) for various lysostaphin variants in Tris. For purposes of comparison, the lytic enzyme PlySs2 (from S. suis) was utilized as a positive control in both N-terminal histidine tagged (i.e., PDySs2NH) and C-terminal histidine tagged (PGySs2CH) forms. As shown in FIG. 13, PDySs2NH and PlySs2Ch were active against S. suis MVS2 in 50 mM Tris-HCl pH8.0.

FIG. 13 shows a turbidity assay in S. pseudintermedius (50 mM Tris, pH 8) for various lysostaphin variants. The results were broken into 4 groups as shown in FIG. 13. For purposes of comparison, the lytic enzyme PlySs2 (from S. suis) was utilized as a positive

| | | Short | | Activity | In Silico |
|---|---|---|---|---|---|
| SEQ | Reason | Name | Variant | Rank | Stability |
| | | natLss | Wild Type | 8 | |
| G²⁵INGG²⁹ | Deamidation | NH1b | G25T | 1 | |
| G²⁵INGG²⁹ | Deamidation | NH1c | N27E | 11 | |
| G²⁵INGG²⁹ | Deamidation | NH1d | N27T | 10 | Destabilize |
| G²⁵INGG²⁹ | Cleavage | NH1e | G29S | 2 | Destabilize |
| G²⁵INGG²⁹ | Deamidation | NH1f | N27D | 12 | |
| AG¹⁴³G¹⁴⁴ | Cleavage | NH2a | G143S | 3 | Stabilize |
| AG¹⁴³G¹⁴⁴ | Cleavage | NH2b | G144Q | 6 | |
| N¹⁵⁷ K¹⁵⁸ | Cleavage | NH3a | K158Q | 4 | |
| N¹⁵⁷ K¹⁵⁸ | Cleavage | NH3b | N157D | 5 | Stabilize |
| G²⁵INGG²⁹, | Glycosylation, | NHall | N27T/ | 9 | N27T: |
| NST¹²⁶, | Cleavage, | | G29S/ | | destabilize |
| AG¹⁴³G¹⁴⁴, | Deamidation | | T126N/ | | T126N, |
| N¹⁵⁷ K¹⁵⁸, | | | G143S/ | | G143S, |
| N K S²³³ | | | N157D/ | | N157D: |
| | | | S233A | | stabilize |
| | Single | CatD | Catalytic | 7 | |
| | domain | | Domain | | |

control in both N-terminal histidine tagged (i.e., PlySs2NH) and C-terminal histidine tagged (PlySs2CH) forms. Further, native lysostaphin (natLss) and N-terminal histidine tagged lysostaphin (LssNH) were also included. PlySs2 is a Streptococcus suis phage lytic enzyme capable of killing Streptococcus bacteria including S. suis in vitro and in vivo in animal models. See Gilmer et al., “Novel bacteriophage lysin with broad lytic activity protects against mixed infection by Streptococcus pyogenes and methicillin-resistant Staphylococcus aureus,” Antimicrob Agents Chemother. 57(6):2743-50 (2013) and Gilmer et al., “The Phage Lysin PlySs2 Decolonizes Streptococcus suis from Murine Intranasal Mucosa,” PLoS One 12(1):e0169180 (2017). PlySs2 has high killing activity against multiple serotypes and strains of S, suis, and demonstrates broad killing activity against Streptococcus and Staphylococcus, including Streptococcus pneumonia and Staphylococcus aureus (WO 2012/145630, published Oct. 26, 2012; U.S. Pat. No. 9,034,322),

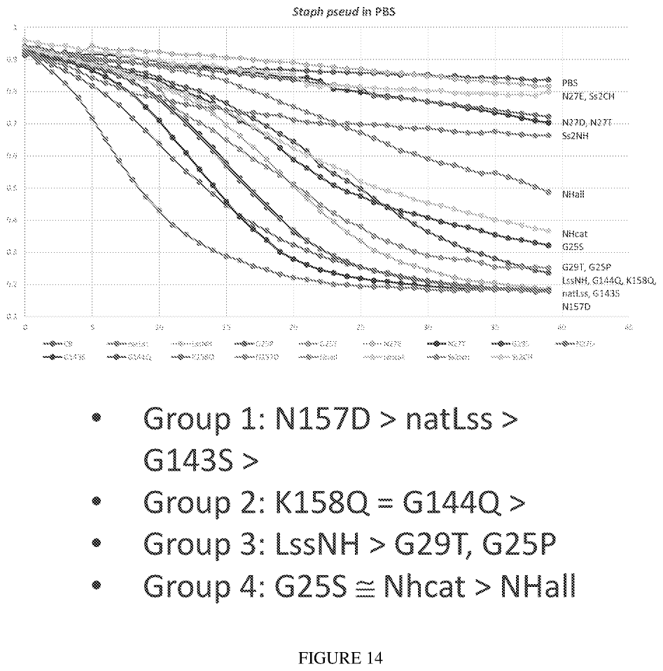

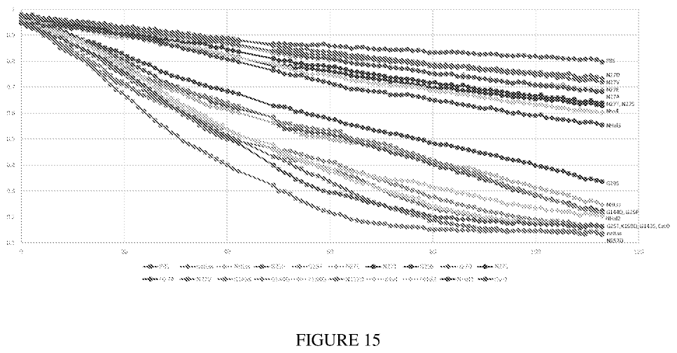

FIG. 14 shows a turbidity assay in S. pseudintermedius (PBS) for various lysostaphin variants. The results were broken into 4 groups as shown in FIG. 15. For purposes of comparison, native lysostaphin (natLss) and N-terminal histidine tagged lysostaphin (LssNH) were also included.

FIG. 15 shows a turbidity assay in S. aureus (50 mM, Tris, pH 8.0) for various lysostaphin variants. For purposes of comparison, native lysostaphin (natLss) and N-terminal histidine tagged lysostaphin (LssNH) were also included.

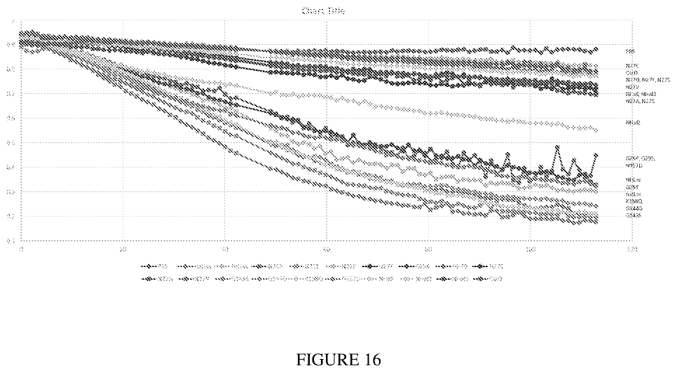

FIG. 16 shows a turbidity assay in S. aureus (PBS) for various lysostaphin variants. For purposes of comparison, native lysostaphin (natLss) and N-terminal histidine tagged lysostaphin (LssNH) were also included.

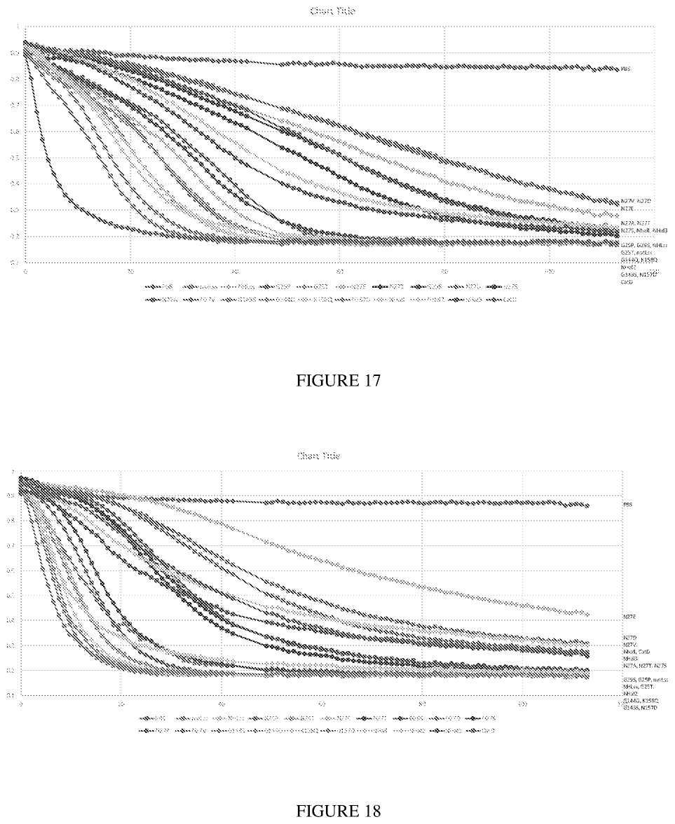

FIG. 17 shows a turbidity assay in S. pseudintermedius (50 mM, Tris, pH 8.0) for various lysostaphin variants. For purposes of comparison, native lysostaphin (natLss) and N-terminal histidine tagged lysostaphin (LssNH) were also included.

FIG. 18 shows a turbidity assay in S. pseudintermedius (PBS) for various lysostaphin variants. For purposes of comparison, native lysostaphin (natLss) and N-terminal histidine tagged lysostaphin (LssNH) were also included.

### Example 4: Stability Analysis of Lysostaphin Variants after 17 Days

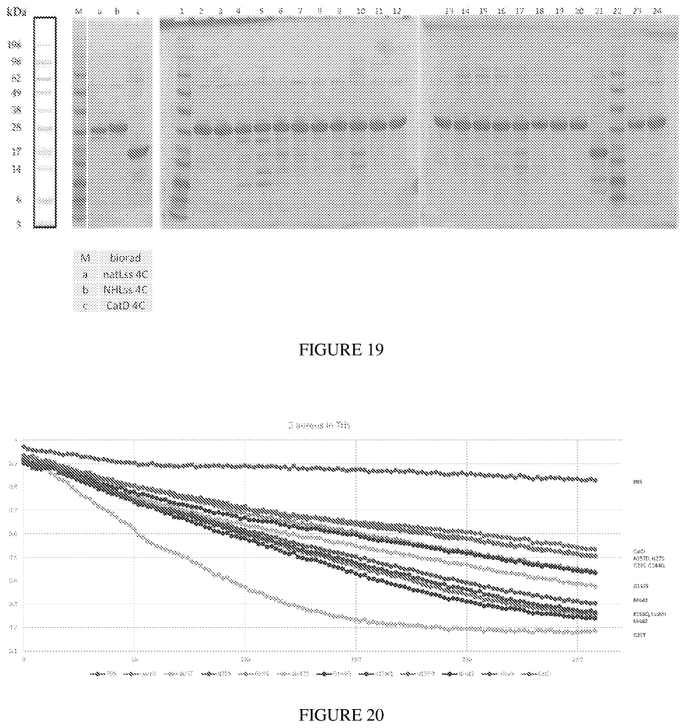

The instant example evaluates the stability of various lysostaphin variants. Aliquots of 18 samples stored at −80° C., 4° C., RT (22-25° C.), 37° C. Each sample was diluted to 1 mg/ml with PBS. An SDS-PAGE analysis was performed (10 μg each loaded into each well). The results of the SDS-PAGE analysis are shown in FIG. 19.

Further, several turbidity assays in S. aureus and in S. pseudintermedius (in Tris and PBS) were performed as follows.

FIG. 20 shows activity at day 17 at 37° C. in S. aureus (Tris). For purposes of comparison, N-terminal histidine tagged lysostaphin (LssNH) was also included.

FIG. 21 shows activity at day 17 at 37° C. in S. aureus (PBS). For purposes of comparison, N-terminal histidine tagged lysostaphin (LssNH) was also included.

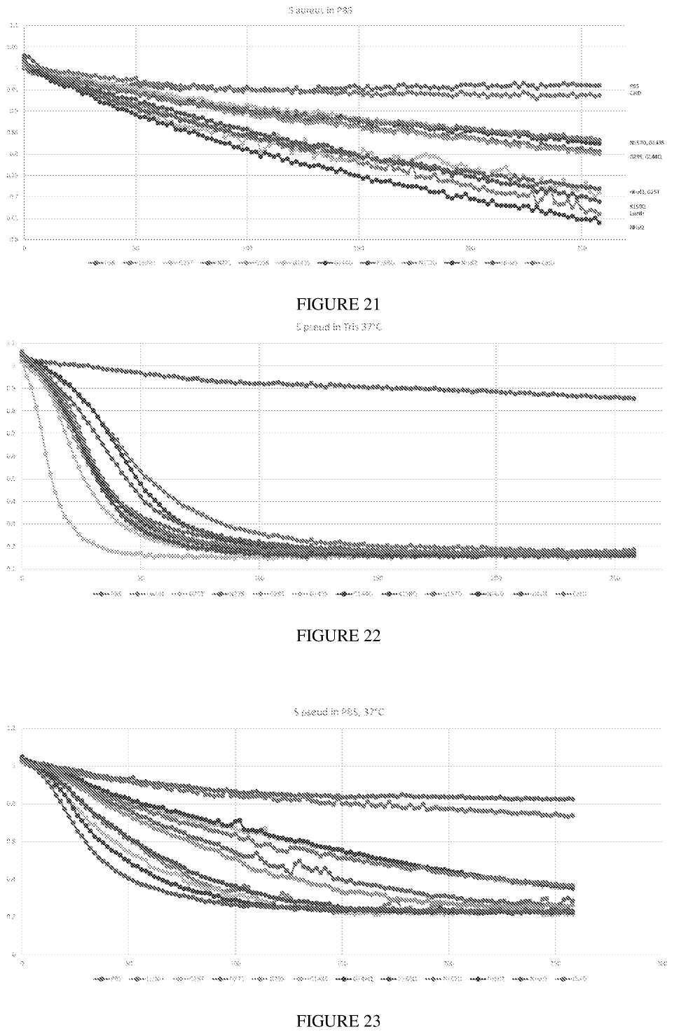

FIG. 22 shows activity at day 17 at 37° C. in S. pseudintermedius (Tris). For purposes of comparison, N-terminal histidine tagged lysostaphin (LssNH) was also included.

FIG. 23 shows activity at day 17 at 37° C. in S. pseudintermedius (PBS). For purposes of comparison, N-terminal histidine tagged lysostaphin (LssNH) was also included.

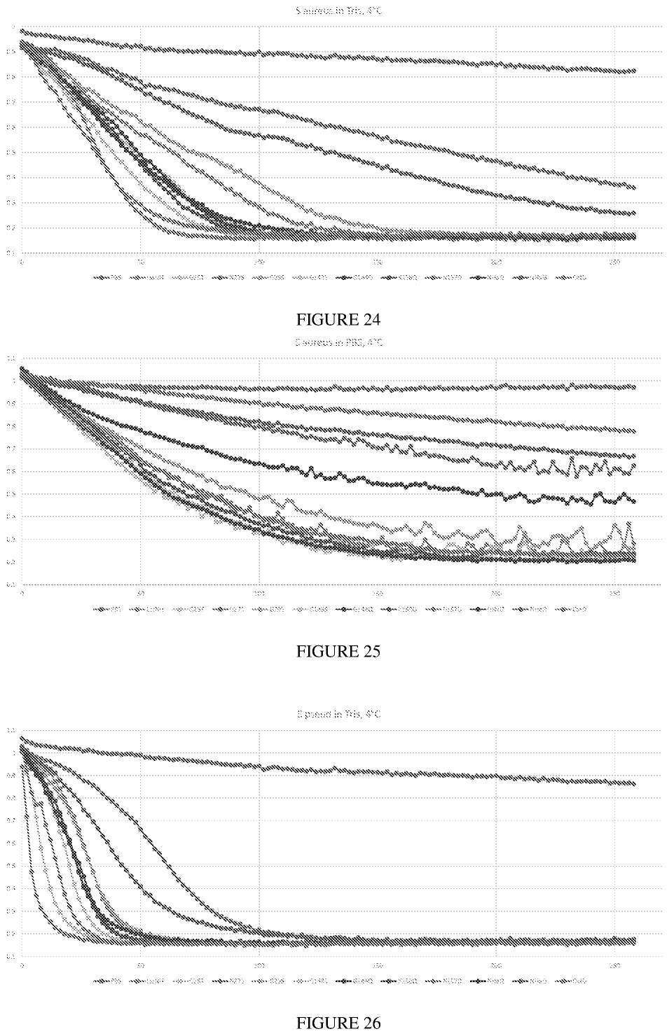

FIG. 24 shows activity at 4° C. in S. aureus (Tris). For purposes of comparison, N-terminal histidine tagged lysostaphin (LssNH) was also included.

FIG. 25 shows activity at 4° C. in S. aureus (PBS). For purposes of comparison, N-terminal histidine tagged lysostaphin (LssNH) was also included.

FIG. 26 shows activity at 4° C. in S. pseudintermedius (Tris). For purposes of comparison, N-terminal histidine tagged lysostaphin (LssNH) was also included.

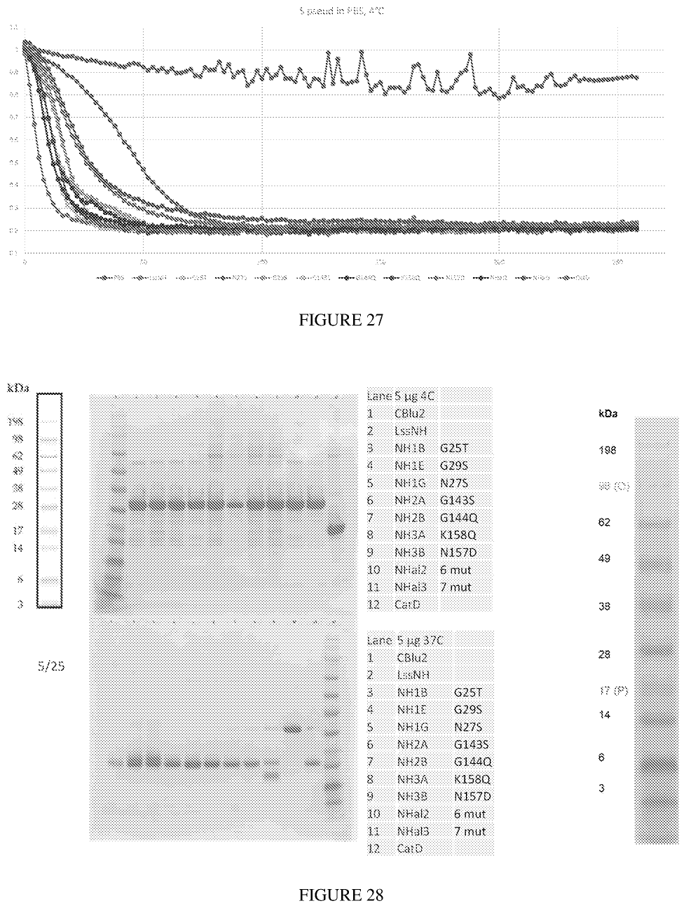

FIG. 27 shows activity at 4° C. in S. pseudintermedius (PBS). For purposes of comparison, N-terminal histidine tagged lysostaphin (LssNH) was also included.

### Example 5: Stability Analysis of Lysostaphin Variants after 32 Days

The instant example evaluates the stability of various lysostaphin variants after 32 days. An SDS-PAGE analysis was performed and the results of the SDS-PAGE analysis are shown in FIG. 28.

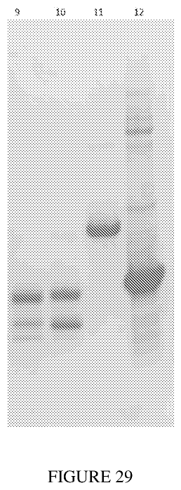

FIG. 29 shows an SDS-PAGE analysis performed on samples stored at room temperature after 32 days. In lane 9, N-terminal histidine tagged lysostaphin (LssNH) is shown. In lane 10, Lss-NHal2 is shown. In lane 11, Lss-NHal3 is shown. In lane 12, Lss-NHCatD is shown. As demonstrated in lane 11, Lss-NHal3 did not demonstrate degradation.

Further, several turbidity assays in S. aureus and in S. pseudintermedius (in Tris and PBS) were performed as follows.

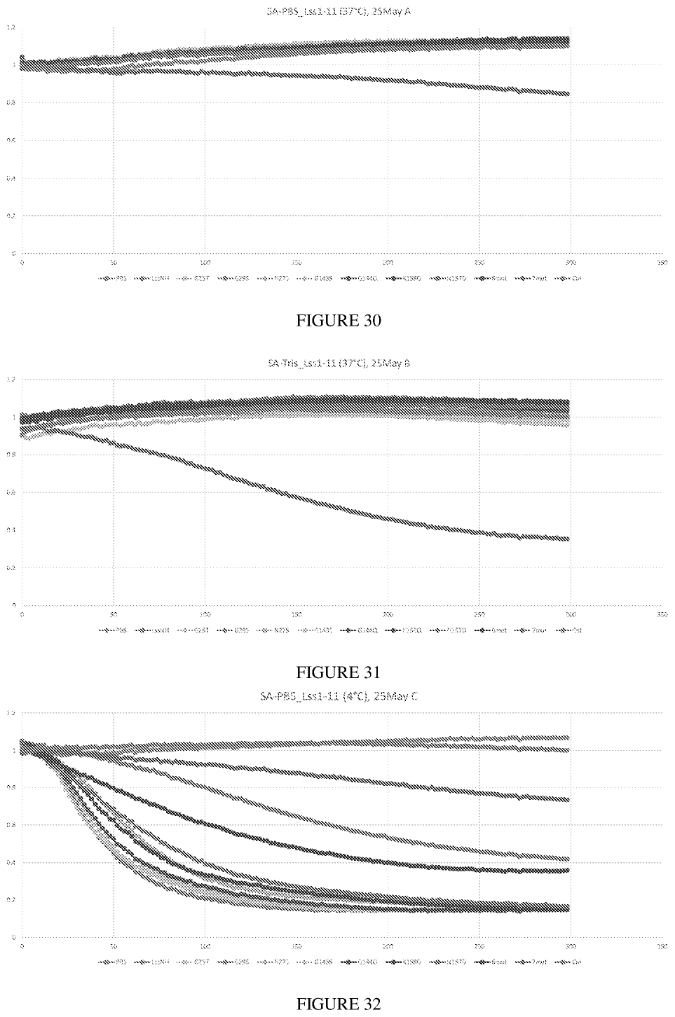

FIG. 30 shows activity at 37° C. in S. aureus (PBS). For purposes of comparison, N-terminal histidine tagged lysostaphin (LssNH) was also included.

FIG. 31 shows activity at 37° C. in S. aureus (Tris). For purposes of comparison, N-terminal histidine tagged lysostaphin (LssNH) was also included.

FIG. 32 shows activity at 4° C. in S. aureus (PBS). For purposes of comparison, N-terminal histidine tagged lysostaphin (LssNH) was also included.

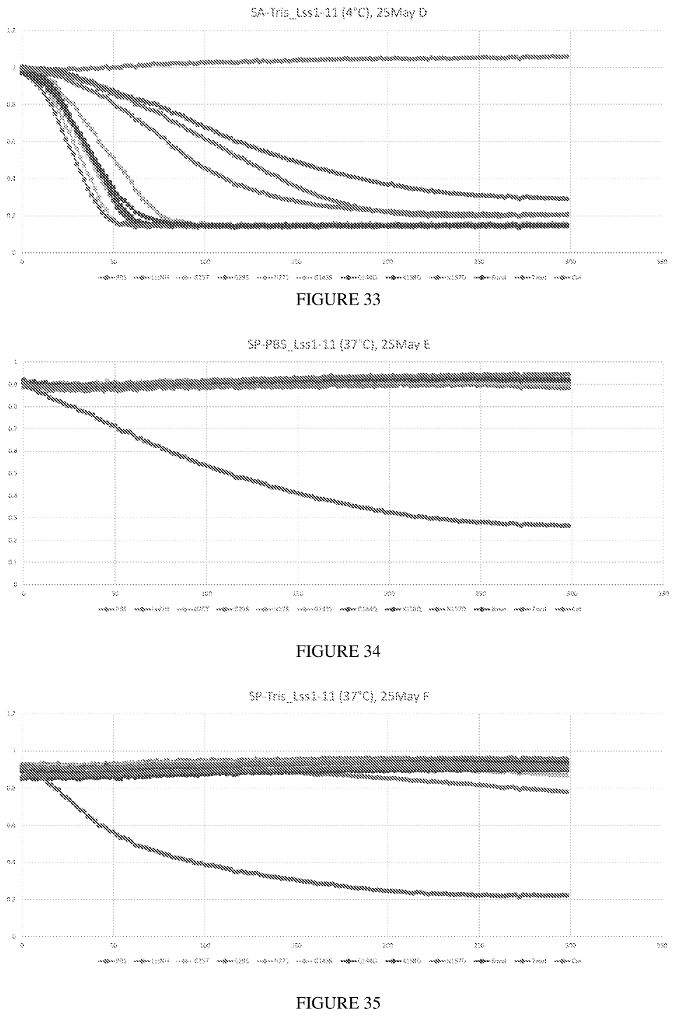

FIG. 33 shows activity at 4° C. in S. aureus (Tris). For purposes of comparison, N-terminal histidine tagged lysostaphin (LssNH) was also included.

FIG. 34 shows activity at 37° C. in S. pseudintermedius (PBS). For purposes of comparison, N-terminal histidine tagged lysostaphin (LssNH) was also included.

FIG. 35 shows activity at 37° C. in S. pseudintermedius (Tris). For purposes of comparison, N-terminal histidine tagged lysostaphin (LssNH) was also included.

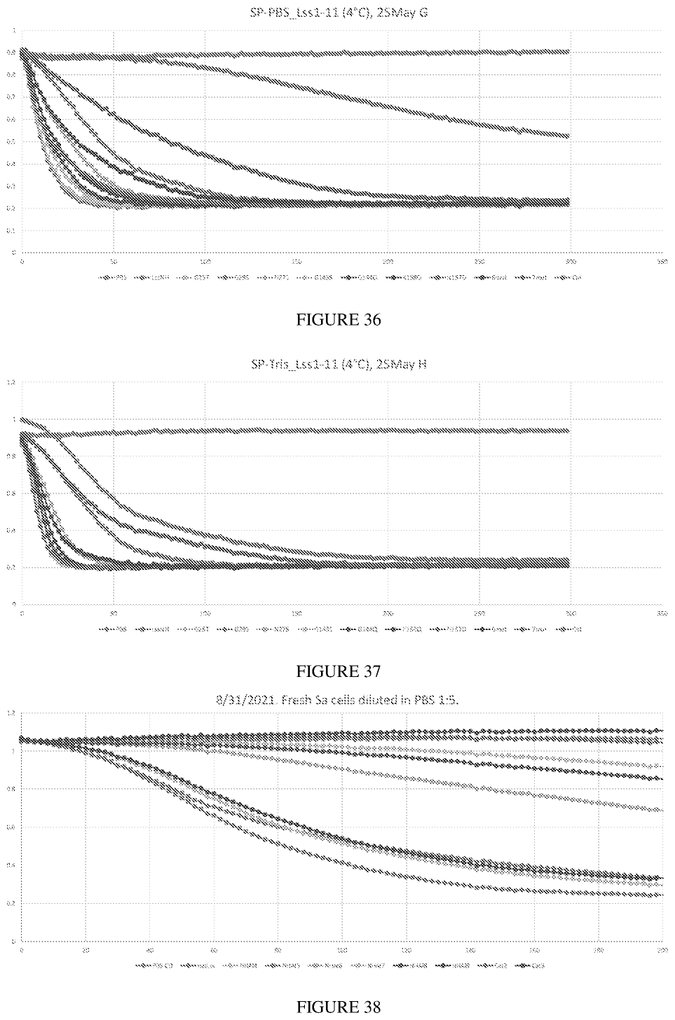

FIG. 36 shows activity at 4° C. in S. pseudintermedius (PBS). For purposes of comparison, N-terminal histidine tagged lysostaphin (LssNH) was also included.

FIG. 37 shows activity at 4° C. in S. pseudintermedius (Tris). For purposes of comparison, N-terminal histidine tagged lysostaphin (LssNH) was also included.

FIG. 38 shows a turbidity assay performed using fresh S. aureus cells diluted 1:5 with PBS. All were diluted with PBS to 50 μg/ml (based on A280).

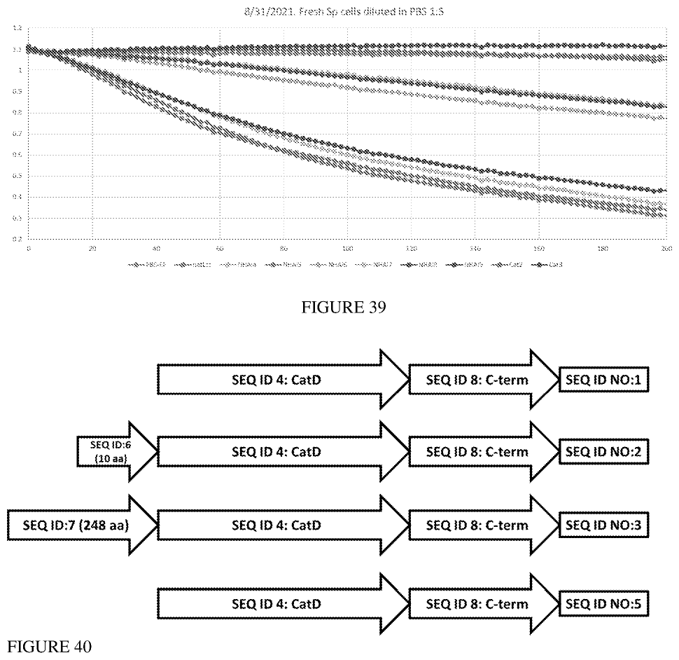

FIG. 39 shows a turbidity assay performed using fresh S. pseudintermedius cells diluted 1:5 with PBS. All were diluted with PBS to 50 μg/ml (based on A280).

What is claimed is:

1. A lysostaphin variant comprising a lysostaphin sequence of SEQ ID NO:5 and comprising at least one amino acid mutation selected from the group consisting of G25P, G25S, G25T, N27E, N27T, N27D, N27V, N27S, N27A, G29S, G29T, G56S, G62S, T126N, G143S, and S233A.

2. The lysostaphin variant of claim 1, wherein the lysostaphin variant has a lower amount of degradation compared to wild type lysostaphin.

3. The lysostaphin variant of claim 1, wherein said variant comprises a polypeptide having the amino acid sequence of any one of SEQ ID NOs:9 to 108.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A pharmaceutical composition comprising the lysostaphin sequence of SEQ ID NO:5 and comprising at least one amino acid mutation selected from the group consisting of G25 P, G25S, G25T, N27E, N27T, N27D, N27V, N27S, N27A, G29S, G29T, G56S, G62S, T126N, G143S, and S233A.

17. The pharmaceutical composition of claim 16, wherein said pharmaceutical composition comprises one or more carriers selected from the group consisting of saline, glucose, alcohols, glycols, esters, amides, polymers, and a combination thereof.

18. The pharmaceutical composition of claim 16, wherein the pharmaceutical composition comprises a second therapeutic agent.

19. A method of treating an infection in an animal, said method comprising the steps of administering a lysostaphin variant or a pharmaceutical composition thereof comprising a lysostaphin sequence of SEQ ID NO:5 and comprising at least one amino acid mutation selected from the group consisting of G25P, G255, G25T, N27E N27T N27D, N27V, N27S, N27A G29S, G29T, G565 G62S T126N G143S and S233A, wherein the lysostaphin variant or pharmaceutical composition thereof reduces one or more symptoms associated with the infection in the animal.

20. The method of claim 19, further comprising administering a second therapeutic agent selected from an antibiotic, an antimicrobial peptide (AMPs) a biocide, a lysin, or antiseptic agent.

21. The method of claim 19, wherein the infection is caused by a Gram-positive bacterium.

22. The method of claim 21, wherein said bacterium is a Methicillin Resistant Staphylococcus aureus (MRSA) bacterium, a Vancomycin Intermediate Staphylococcus aureus (VISA) bacterium, or a Vancomycin Resistant Staphylococcus aureus (VRSA) bacterium.

23. The method of claim 19, wherein said animal is selected from the group consisting of humans, ruminant animals, poultry, swine, canines, felines, Mustelidae family, and Leporidae family.

24. The method of claim 19, wherein the lysostaphin variant or pharmaceutical composition is administered at an amount of between 0.001 to 1000 mg of the lysostaphin variant or is administered at a dose of about 0.001 to about 1000 mg of the lysostaphin variant per kg of animal body weight and is provided once daily, twice daily, or three times daily.

25. (canceled)

26. A recombinant nucleic acid vector comprising a transcription promoter, a nucleic acid sequence encoding a lysostaphin polypeptide sequence of SEQ ID NO: 5 and comprising at least one amino acid mutation selected from the group consisting of G25P, G25S, G25T, N27E, N27T, N27D, N27V, N27S, N27A, G29S, G29T, G56S, G62S, T126N, G143S, and S233A, and a transcriptional terminator.

27. A method of producing a lysostaphin variant comprising a lysostaphin sequence of SEQ LD NO:4 and comprising at least one amino acid mutation selected from the group consisting of G25P, G25S, G25T, N27E, N27T, N27D, N27V, N27S, N27A, G129S, G29T, G56S, G62S, and T126N, said method comprising inducing expression of the lysostaphin polypeptide from the recombinant nucleic acid vector a transcription promoter, a nucleic acid sequence encoding a lysostaphin polypeptide sequence of SE ID NO:5 and comprising at least one amino acid mutation selected from the group consisting of G25P, G25S, G25T N27E N27T, N27D N27V, N27S, N27A, G29S, G29T, G56S, G62S, T126N, G143S, and S233A, and a transcriptional terminator in a cell.

28. (canceled)

29. The method of claim 19, wherein said lysostaphin variant comprises a polypeptide having the amino acid sequence of any one of SEQ ID NOs:9 to 108.

30. The method of claim 19, wherein said lysostaphin variant comprises a polypeptide having the amino acid sequence of any one of SEQ ID NOs:11, 14, 23; 25, and 26.

31. The lysostaphin variant of claim 3, wherein said lysostaphin variant comprises a polypeptide having the amino acid sequence of any one of SEQ ID NOs:11, 14, 23; 25, and 26.

32. The pharmaceutical composition of claim 16, wherein said lysostaphin variant comprises a polypeptide having the amino acid sequence of any one of SEQ ID NOs:11, 14, 23; 25, and 26.

33. The pharmaceutical composition of claim 16, wherein said pharmaceutical composition if formulated for oral, topical, or parenteral administration.

34. The pharmaceutical composition of claim 18, wherein said second therapeutic agent comprises selected from an antibiotic, an antimicrobial peptide (AMP), a biocide, a lysin, or antiseptic agent.

35. The recombinant nucleic acid vector of claim 26, wherein said nucleic acid sequence encoding a lysostaphin variant comprising a polypeptide having the amino acid sequence of any one of SEQ ID NOs:9 to 108.


SEQUENCE LISTING (127 sequences)

SEQ ID NO: 1 - protein, 245 aa, Staphylococcus simulans
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 2 - protein, 255 aa, Staphylococcus simulans
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 3 - protein, 493 aa, Staphylococcus simulans
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 4 - protein, 142 aa, Staphylococcus simulans
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 5 - protein, 245 aa, synthetic construct
  source 1..245: note = Recombinant engineered Lysostaphin from Staphylococcus simulans
  VARIANT 25: note = X can be Glycine, Proline, Serine, or Threonine
  VARIANT 29: note = X can be Glycine, Serine, Threonine
  VARIANT 56: note = X can be G or Serine
  VARIANT 61: note = X can be G or Serine
  VARIANT 142: note = X can be A or S
  VARIANT 143: note = X can be G or S
  VARIANT 144: note = X can be G or Q
  VARIANT 157: note = X can be N or D
  VARIANT 233: note = X can be S or A
  VARIANT 126: note = X can be T or N
  VARIANT 27: note = X can be Asparagine, Glutamic Acid, Phenylalanine, Aspartic Acid, valine, serine, or alanine
ATHEHSAQWLNNYKKGYGYGPYPLXIXGXMHYGVDFFMNIGTPVKAISSGKIVEAXWSNYXGGNQIGLIENDGVHRQWYMHLSKYNVKVGDYVKAGQIIGWSGSTGYSTAPHLHFQRMVNSFSNSXAQDPMPFLKSAGYGKXXXTVTPTPNTGWKTXKYGTLYKSESASFTPNTDIITRTTGPFRSMPQSGVLKAGQTIHYDEVMKQDGHVWVGYTGNSGQRIYLPVRTWNKXTNTLGVLWGTIK

SEQ ID NO: 6 - protein, 10 aa, Staphylococcus simulans
ASGFYVSGTA

SEQ ID NO: 7 - protein, 248 aa, Staphylococcus simulans
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRA

SEQ ID NO: 8 - protein, 103 aa, Staphylococcus simulans
  VARIANT 1: note = X can be G or S
  VARIANT 2: note = X can be G or Q
  VARIANT 15: note = X can be N or D
  VARIANT 91: note = X can be S or A
XXTVTPTPNTGWKTXKYGTLYKSESASFTPNTDIITRTTGPFRSMPQSGVLKAGQTIHYDEVMKQDGHVWVGYTGNSGQRIYLPVRTWNKXTNTLGVLWGTIK

SEQ ID NO: 9 - protein, 142 aa, synthetic construct
ATHEHSAQWLNNYKKGYGYGPYPLPINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 10 - protein, 142 aa, synthetic construct
ATHEHSAQWLNNYKKGYGYGPYPLSINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 11 - protein, 142 aa, synthetic construct
ATHEHSAQWLNNYKKGYGYGPYPLTINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 12 - protein, 142 aa, synthetic construct
ATHEHSAQWLNNYKKGYGYGPYPLGIEGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 13 - protein, 142 aa, synthetic construct
ATHEHSAQWLNNYKKGYGYGPYPLGITGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 14 - protein, 142 aa, synthetic construct
ATHEHSAQWLNNYKKGYGYGPYPLGIDGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 15 - protein, 142 aa, synthetic construct
ATHEHSAQWLNNYKKGYGYGPYPLGIVGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 16 - protein, 142 aa, synthetic construct
ATHEHSAQWLNNYKKGYGYGPYPLGISGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 17 - protein, 142 aa, synthetic construct
ATHEHSAQWLNNYKKGYGYGPYPLGIAGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 18 - protein, 142 aa, synthetic construct
ATHEHSAQWLNNYKKGYGYGPYPLGINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 19 - protein, 142 aa, synthetic construct
ATHEHSAQWLNNYKKGYGYGPYPLGINGTMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 20 - protein, 142 aa, synthetic construct
ATHEHSAQWLNNYKKGYGYGPYPLGINGGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 21 - protein, 142 aa, synthetic construct
ATHEHSAQWLNNYKKGYGYGPYPLGINGGMHYGVDFFMNIGTPVKAISSGKIVEAGWSNYSGGNQIGLIENDGVHRQWYMHLSKYNVKVGDYVKAGQIIGWSGSTGYSTAPHLHFQRMVNSFSNSTAQDPMPFLKSAGYGKA

SEQ ID NO: 22 - protein, 142 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKA

SEQ ID NO: 23 - protein, 103 aa, synthetic construct
SGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 24 - protein, 103 aa, synthetic construct
GQTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 25 - protein, 103 aa, synthetic construct
GGTVTPTPNTGWKTDKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 26 - protein, 103 aa, synthetic construct
GGTVTPTPNTGWKTNQYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 27 - protein, 103 aa, synthetic construct
GGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 28 - protein, 143 aa, synthetic construct
ATHEHSAQWLNNYKKGYGYGPYPLGITGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKAS

SEQ ID NO: 29 - protein, 143 aa, synthetic construct
ATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKAS

SEQ ID NO: 30 - protein, 143 aa, synthetic construct
ATHEHSAQWLNNYKKGYGYGPYPLTISGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKAS

SEQ ID NO: 31 - protein, 143 aa, synthetic construct; identical to SEQ ID NO: 29

SEQ ID NO: 32 - protein, 143 aa, synthetic construct
ATHEHSAQWLNNYKKGYGYGPYPLTINSGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSNAQDPMPFLKSAGYGKAS

SEQ ID NO: 33 - protein, 143 aa, synthetic construct
ATHEHSAQWLNNYKKGYGYGPYPLTINSGMHYGVDFFMNIGTPVKAISSGKIVEASWSNYGSGNQIGLIENDGVHRQWYMHLSKYNVKVGDYVKAGQIIGWSGSTGYSTAPHLHFQRMVNSFSNSNAQDPMPFLKSAGYGKAS

SEQ ID NO: 34 - protein, 245 aa, synthetic construct
  VARIANT 25: note = G25P
ATHEHSAQWLNNYKKGYGYGPYPLPINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 35 - protein, 245 aa, synthetic construct
ATHEHSAQWLNNYKKGYGYGPYPLSINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 36 - protein, 245 aa, synthetic construct
ATHEHSAQWLNNYKKGYGYGPYPLTINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 37 - protein, 245 aa, synthetic construct
ATHEHSAQWLNNYKKGYGYGPYPLGIEGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 38 - protein, 245 aa, synthetic construct
ATHEHSAQWLNNYKKGYGYGPYPLGITGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 39 - protein, 245 aa, synthetic construct
ATHEHSAQWLNNYKKGYGYGPYPLGIDGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 40 - protein, 245 aa, synthetic construct
ATHEHSAQWLNNYKKGYGYGPYPLGIVGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 41 - protein, 245 aa, synthetic construct
ATHEHSAQWLNNYKKGYGYGPYPLGISGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 42 - protein, 245 aa, synthetic construct
ATHEHSAQWLNNYKKGYGYGPYPLGIAGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 43 - protein, 245 aa, synthetic construct
ATHEHSAQWLNNYKKGYGYGPYPLGINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 44 - protein, 245 aa, synthetic construct
ATHEHSAQWLNNYKKGYGYGPYPLGINGTMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 45 - protein, 245 aa, synthetic construct
ATHEHSAQWLNNYKKGYGYGPYPLGINGGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 46 - protein, 245 aa, synthetic construct
ATHEHSAQWLNNYKKGYGYGPYPLGINGGMHYGVDFFMNIGTPVKAISSGKIVEAGWSNYSGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 47 - protein, 245 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 48 - protein, 245 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 49 - protein, 245 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGQTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 50 - protein, 245 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTDKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 51 - protein, 245 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNQYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 52 - protein, 245 aa, synthetic construct
ATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 53 - protein, 245 aa, synthetic construct
ATHEHSAQWLNNYKKGYGYGPYPLGITGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 54 - protein, 245 aa, synthetic construct
ATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 55 - protein, 245 aa, synthetic construct
ATHEHSAQWLNNYKKGYGYGPYPLTISGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 56 - protein, 245 aa, synthetic construct
ATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 57 - protein, 245 aa, synthetic construct
ATHEHSAQWLNNYKKGYGYGPYPLTINSGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 58 - protein, 245 aa, synthetic construct
ATHEHSAQWLNNYKKGYGYGPYPLTINSGMHYGVDFFMNIGTPVKAISSGKIVEASWSNYGSGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 59 - protein, 255 aa, synthetic construct
ASGFYVSGTAATHEHSAQWLNNYKKGYGYGPYPLPINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 60 - protein, 255 aa, synthetic construct
ASGFYVSGTAATHEHSAQWLNNYKKGYGYGPYPLSINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 61 - protein, 255 aa, synthetic construct
ASGFYVSGTAATHEHSAQWLNNYKKGYGYGPYPLTINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 62 - protein, 255 aa, synthetic construct
ASGFYVSGTAATHEHSAQWLNNYKKGYGYGPYPLGIEGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 63 - protein, 255 aa, synthetic construct
ASGFYVSGTAATHEHSAQWLNNYKKGYGYGPYPLGITGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 64 - protein, 255 aa, synthetic construct
ASGFYVSGTAATHEHSAQWLNNYKKGYGYGPYPLGIDGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 65 - protein, 255 aa, synthetic construct
ASGFYVSGTAATHEHSAQWLNNYKKGYGYGPYPLGIVGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 66 - protein, 255 aa, synthetic construct
ASGFYVSGTAATHEHSAQWLNNYKKGYGYGPYPLGISGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 67 - protein, 255 aa, synthetic construct
ASGFYVSGTAATHEHSAQWLNNYKKGYGYGPYPLGIAGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 68 - protein, 255 aa, synthetic construct
ASGFYVSGTAATHEHSAQWLNNYKKGYGYGPYPLGINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 69 - protein, 255 aa, synthetic construct
ASGFYVSGTAATHEHSAQWLNNYKKGYGYGPYPLGINGTMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 70 - protein, 255 aa, synthetic construct
ASGFYVSGTAATHEHSAQWLNNYKKGYGYGPYPLGINGGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 71 - protein, 255 aa, synthetic construct
ASGFYVSGTAATHEHSAQWLNNYKKGYGYGPYPLGINGGMHYGVDFFMNIGTPVKAISSGKIVEAGWSNYSGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 72 - protein, 255 aa, synthetic construct
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 73 - protein, 255 aa, synthetic construct
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 74 - protein, 255 aa, synthetic construct
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGQTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 75 - protein, 255 aa, synthetic construct
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTDKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 76 - protein, 255 aa, synthetic construct
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNQYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 77 - protein, 255 aa, synthetic construct
ASGFYVSGTAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 78 - protein, 255 aa, synthetic construct
ASGFYVSGTAATHEHSAQWLNNYKKGYGYGPYPLGITGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 79 - protein, 255 aa, synthetic construct
ASGFYVSGTAATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 80 - protein, 255 aa, synthetic construct
ASGFYVSGTAATHEHSAQWLNNYKKGYGYGPYPLTISGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 81 - protein, 255 aa, synthetic construct
ASGFYVSGTAATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 82 - protein, 255 aa, synthetic construct
ASGFYVSGTAATHEHSAQWLNNYKKGYGYGPYPLTINSGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 83 - protein, 255 aa, synthetic construct
ASGFYVSGTAATHEHSAQWLNNYKKGYGYGPYPLTINSGMHYGVDFFMNIGTPVKAISSGKIVEASWSNYGSGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 84 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLPINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 85 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLSINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 86 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLTINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 87 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGIEGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 88 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGITGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 89 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGIDGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 90 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGIVGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 91 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGISGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 92 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGIAGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 93 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 94 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGTMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 95 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 96 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYSGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 97 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 98 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 99 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGQTVTPTPNTGWKTNKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 100 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTDKYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 101 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNQYGTLYKSESASFTPNTDIITRTTGPFRSMPQSGVLKAGQTIHYDEVMKQDGHVWVGYTGNSGQRIYLPVRTWNKSTNTLGVLWGTIK

SEQ ID NO: 102 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGINGGMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSTAQDPMPFLKSAGYGKAGGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 103 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLGITGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 104 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 105 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLTISGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTDKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 106 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLTINGSMHYGVDFFMNIGTPVKAISSGKIVEAGWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 107 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLTINSGMHYGVDFFMNIGTPVKAISSGKIVEASWSNYGGGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 108 - protein, 493 aa, synthetic construct
MKKTKNNYYTRPLAIGLSTFALASIVYGGIQNETHASEKSNMDVSKKVAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKAPVENTAEVETSKALVQNRTALRAATHEHSAQWLNNYKKGYGYGPYPLTINSGMHYGVDFFMNIGTPVKAISSGKIVEASWSNYGSGNQIGLIENDGVHRQWYMHLSKYNVKVGDYVKAGQIIGWSGSTGYSTAPHLHFQRMVNSFSNSNAQDPMPFLKSAGYGKASGTVTPTPNTGWKTNKYGTLYKSESASFTPNTDIITRTTGPFRSMPQSGVLKAGQTIHYDEVMKQDGHVWVGYTGNSGQRIYLPVRTWNKATNTLGVLWGTIK

SEQ ID NO: 109 - protein, 5 aa, Staphylococcus simulans
  MOD_RES 3: note = Deamidation site
GINGG

SEQ ID NO: 110 - DNA, 735 bp, Staphylococcus simulans
  source 1..735: mol_type = other DNA
gctacccacgaacacagcgcgcagtggctgaataactacaagaaaggttacggttatggcccttaccctctcggtatcaacggtggcatgcattatggcgtagacttctttatgaatattggcaccccggttaaagcaatcagctccggtaaaatcgtcgaagcgggttggtccaactacggcggtggcaaccagatcggtctcatcgaaaacgatggtgttcatcgtcaatggtacatgcacctgtccaaatacaacgtgaaagtgggcgactatgtaaaagcaggtcagattatcggttggagcggttctaccggctacagcactgcaccacacctgcatttccagcgtatggttaactccttttctaactctaccgctcaggatccgatgccttttctgaaaagcgccggttacggtaaagcaggtggcactgtaaccccgaccccgaacaccggctggaaaactaacaaatacggcaccctctacaaaagcgaatccgctagcttcaccccaaacactgatattatcacccgtaccacgggtccattccgtagcatgcctcagtccggtgttctgaaagcgggtcagactattcactacgatgaagtcatgaaacaggatggtcatgtttgggtcggctatactggcaactctggccagcgcatttacctcccggtccgtacctggaataaaagcactaacactctgggtgtactgtggggcaccattaaa

SEQ ID NO: 111 - DNA, 735 bp, synthetic construct
  source 1..735: mol_type = other DNA
gcaacccacgaacacagcgcgcagtggctgaataactacaagaaaggttacggttacggtccgtacccgctgccgattaacggcggtatgcactacggtgtagacttctttatgaacatcggtaccccggttaaggcaatcagctctggcaaaatcgtggaagctggctggtctaattacggtggcggtaatcagatcggtctgatcgaaaacgacggcgttcaccgccagtggtacatgcacctgtctaaatacaatgtcaaagtgggcgactacgttaaagctggtcagattatcggctggtctggtagcaccggctactctaccgcaccgcatctccacttccagcgtatggttaactctttctccaattccaccgcccaggacccgatgccgttcctgaagagcgctggctacggcaaagccggcggtactgtcacgccgaccccgaacaccggttggaaaacgaacaaatacggtactctctataaatccgaatctgcgagcttcactcctaacaccgatattatcacccgcaccactggtccgttccgctccatgccgcagtctggtgttctgaaggcgggtcagacgattcactacgatgaagttatgaaacaagacggtcacgtgtgggtgggctatacgggtaactctggtcaacgtatttacctgccggtacgtacttggaacaagagcaccaataccctgggtgtactgtggggcactatcaaa

SEQ ID NO: 112 - DNA, 735 bp, synthetic construct
  source 1..735: mol_type = other DNA
gcgacccacgaacactccgctcagtggctgaacaattacaagaaaggttacggttacggtccttacccgctgaccatcaacggtggcatgcactatggtgttgatttctttatgaacattggtaccccggttaaagcgatttcctctggcaaaattgttgaagccggttggtccaactatggcggtggcaaccagattggcctgatcgagaacgacggcgttcaccgtcagtggtacatgcacctgtccaaatacaacgttaaagttggtgactacgttaaggctggccagattatcggttggagcggctccaccggttactccactgccccacatctgcacttccagcgtatggtgaactccttctccaactctactgcacaggatccgatgccgtttctgaaatctgcgggttacggtaaagctggcggtaccgttacgccgactccgaacactggttggaaaaccaacaaatacggtaccctgtacaaatccgaatccgcttctttcaccccgaacactgacatcattactcgcaccacgggcccgttccgttccatgccacagtctggcgtgctgaaggctggccaaactatccattacgacgaagtcatgaaacaggacggtcatgtctgggtgggttacactggcaacagcggtcaacgtatctacctgccggtgcgtacctggaataaatctaccaacaccctgggcgttctgtggggtactatcaag

SEQ ID NO: 113 - DNA, 735 bp, synthetic construct
  source 1..735: mol_type = other DNA
gctactcatgagcactccgctcagtggctgaataactacaaaaagggctacggctacggtccgtacccgctgggtatcgaaggcggtatgcactacggtgttgactttttcatgaatatcggcaccccggttaaagcaatttcttccggtaaaatcgtcgaagcaggttggagcaactacggcggtggcaaccagatcggcctgatcgaaaacgatggtgttcatcgtcagtggtacatgcatctgagcaaatacaacgttaaagtaggtgactacgtaaaagcgggtcagattatcggttggtctggctctacgggctattccaccgcgccacacctgcacttccagcgcatggtaaattcctttagcaattccactgctcaggacccgatgccattcctgaagagcgcaggctacggtaaagcaggcggtaccgtgaccccgactccgaacaccggctggaaaactaacaaatacggtactctgtataagtctgaaagcgcgtctttcaccccaaacaccgatatcattactcgtaccactggcccgttccgttctatgccgcaatctggtgtgctgaaggcaggtcagactatccactacgacgaagttatgaaacaagatggtcacgtttgggtgggttacaccggtaactccggccagcgtatctacctcccggtacgcacttggaataaatccactaacaccctgggtgtgctgtggggtaccatcaaa

SEQ ID NO: 114 - DNA, 735 bp, synthetic construct
  source 1..735: mol_type = other DNA
gctacccacgaacatagcgcgcagtggctgaataactataagaaaggttacggctacggcccataccctctgggtattaccggcggtatgcattacggcgtggattttttcatgaacattggtaccccggtgaaagctattagctccggtaaaatcgtagaggcgggttggagcaactacggcggtggcaaccagattggcctgattgaaaacgacggcgtgcaccgccagtggtatatgcacctgagcaaatacaacgttaaggttggcgattatgtgaaagctggtcagattatcggctggtccggctctactggttattctacggcgccgcatctgcacttccagcgtatggtgaactctttctctaacagcactgctcaggatccaatgccgttcctgaaatccgcaggctacggcaaggctggtggcaccgtaaccccgaccccgaacaccggttggaaaaccaacaaatacggtactctgtacaaatctgaatccgcaagcttcactcctaacactgacattatcacgcgtactaccggcccgtttcgttctatgccgcagagcggtgttctgaaagctggtcagacgatccactatgacgaagtgatgaaacaggacggtcacgtttgggtcggctacactggtaattccggccagcgcatctatctgccggttcgtacgtggaacaagagcaccaacaccctgggtgttctgtggggcaccatcaaa

SEQ ID NO: 115 - DNA, 735 bp, synthetic construct
  source 1..735: mol_type = other DNA
gcgacccatgaacacagcgcacagtggctgaataactataagaaaggctacggttacggcccgtacccactgggtatcaacggtagcatgcactatggtgtggatttctttatgaacatcggcactcctgtaaaagcaattagctctggcaagattgtcgaagcgggctggtctaactatggtggcggtaaccagatcggtctgatcgaaaatgatggcgttcaccgccaatggtatatgcacctgagcaaatacaacgttaaagtgggcgactacgtgaaagctggccagatcattggctggtctggctctaccggttacagcactgcgccgcacctccacttccagcgcatggtgaacagcttctctaactctaccgctcaggatccgatgccgttcctgaaaagcgccggctacggcaaggctggcggtactgtcacccctaccccaaatacgggctggaaaacgaacaaatacggtacgctgtacaaatccgaaagcgcatccttcaccccaaacaccgacattatcacccgtacgaccggtccgtttcgttctatgccgcagagcggtgttctcaaagcgggtcagactatccactacgatgaagtaatgaagcaggacggccatgtttgggttggttacacgggcaactccggccagcgcatttacctgccggtccgtacttggaataaatccactaacaccctgggtgtgctgtggggcacgattaaa

SEQ ID NO: 116 - DNA, 735 bp, synthetic construct
  source 1..735: mol_type = other DNA
gcgactcacgagcactctgcgcagtggctgaataactataagaaaggttacggttacggcccgtacccgctgggcatcaatggcggtatgcactatggcgtagattttttcatgaacattggtactccggttaaggcaatctcctctggcaaaatcgttgaagctggctggtccaactatggtggcggtaatcagatcggtctgatcgagaacgatggtgtgcatcgccagtggtacatgcatctgagcaaatataacgtgaaagtaggtgattacgtaaaagcaggccagatcattggctggtccggtagcaccggttatagcactgcaccacatctgcacttccagcgtatggttaattctttttctaattctaccgcacaggatcctatgccgttcctgaaaagcgcaggttacggtaaagcctctggcactgtgactccgactccgaacactggttggaaaactaacaagtatggtacgctgtataaatccgaaagcgcttctttcaccccgaacaccgacattatcacccgtaccactggcccgttccgttctatgccgcagtccggtgtcctgaaagccggccagaccatccactacgacgaggtgatgaaacaggacggccatgtttgggtgggttacaccggcaactctggccagcgcatttacctgccggtacgcacctggaacaaaagcactaacaccctcggtgttctgtggggcaccatcaaa

SEQ ID NO: 117 - DNA, 735 bp, synthetic construct
  source 1..735: mol_type = other DNA
gcgactcacgaacactctgcccagtggctgaataactataagaaaggctatggttacggtccgtacccgctgggtattaacggcggtatgcattatggtgtggactttttcatgaacattggtactccggttaaagccatttcttccggcaagatcgtggaagcaggttggtccaattatggtggcggtaaccagattggcctgatcgaaaacgatggtgttcaccgccaatggtatatgcacctgtctaaatacaacgtgaaagtgggcgattacgtcaaggctggtcagattatcggctggtctggtagcacgggctactccaccgcgccgcacctgcacttccaacgtatggttaactctttctctaactccaccgcccaggatccgatgccgttcctgaaatctgcaggttacggtaaggcgggtcagaccgtgaccccgactccgaacaccggctggaagacgaacaaatacggtaccctgtataaaagcgaaagcgctagcttcaccccgaacaccgatattatcacgcgtacgaccggtccgtttcgctctatgccgcaatctggtgttctgaaagctggccagactattcactacgacgaagtgatgaaacaggatggtcacgtgtgggtgggctacaccggcaactctggccaacgcatctacctcccagttcgtacttggaacaaaagcactaacaccctgggcgttctgtggggtaccatcaaa

SEQ ID NO: 118 - DNA, 735 bp, synthetic construct
  source 1..735: mol_type = other DNA
gcgacgcacgagcactctgcccaatggctgaataactacaagaaaggttacggttatggtccgtatccgctgggcatcaacggtggcatgcactacggcgtggatttctttatgaacatcggcaccccggtgaaagcgatcagctctggcaaaatcgttgaagcgggttggtctaactacggtggcggtaaccagatcggcctgattgaaaacgatggcgtgcaccgtcagtggtatatgcacctgtccaaatacaacgtaaaagtaggcgactatgttaaagccggccagattatcggctggtctggctccactggctactccactgctccgcatctgcatttccagcgtatggtcaactccttttccaactctactgcgcaagatccgatgccgtttctgaagtctgcgggctacggtaaagcgggcggtactgtgactccgacgccaaacaccggttggaaaaccaaccaatatggtacgctgtacaaaagcgagagcgcgtccttcaccccgaataccgatattatcactcgcaccacgggcccgtttcgctccatgccgcagagcggtgtgctgaaagctggtcagaccatccactacgacgaagtaatgaaacaggatggtcatgtttgggtcggttataccggtaacagcggccagcgtatctatctgccggttcgcacctggaacaagtctactaataccctgggtgtgctgtggggtaccatcaag

SEQ ID NO: 119 - DNA, 735 bp, synthetic construct
  source 1..735: mol_type = other DNA
gccacccacgaacatagcgcacagtggctgaataactacaagaaaggttatggttacggtccgtaccctctgggcatcaacggtggcatgcattacggcgtagattttttcatgaacatcggtaccccggttaaagcgatttcttccggtaagatcgtcgaagcgggctggagcaactacggtggcggtaaccagatcggcctgatcgaaaacgacggcgttcatcgtcagtggtacatgcatctgtctaaatacaacgttaaagtcggcgactacgtcaaagccggtcagatcattggctggtctggctctaccggctactctacggctccgcatctgcacttccagcgtatggttaactccttcagcaactccaccgctcaggatccgatgccgtttctgaaatccgcaggttacggcaaagccggcggtaccgttacccctactccgaacaccggttggaaaactgacaaatacggtaccctgtataaatccgaaagcgctagctttacgcctaacaccgacattatcacccgcacgaccggcccattccgcagcatgccgcagtccggcgtgctgaaagcgggtcagactattcactacgacgaagtgatgaagcaggatggtcacgtttgggtcggttacactggtaattctggccagcgtatctatctgccggtacgtacctggaacaaatctaccaacacgctgggcgtcctgtggggcaccatcaaa

SEQ ID NO: 120 - DNA, 735 bp, synthetic construct
  source 1..735: mol_type = other DNA
gcgacccacgaacactctgctcaatggctgaacaattataagaaaggctacggttacggcccgtacccgctgggtattaccggttctatgcattacggtgttgatttctttatgaacatcggtactccggttaaagcgatttctagcggtaagatcgtggaggcgggctggagcaactacggtggcggtaaccaaattggtctgattgaaaatgacggtgttcaccgtcagtggtatatgcacctgtctaagtataacgttaaagttggtgactatgttaaagccggtcagattatcggctggagcggcagcaccggctactctactgccccgcacctgcacttccagcgtatggtcaactctttctccaactctaatgcgcaggacccgatgccgttcctgaaatccgctggctatggcaaagctagcggcaccgtaactccaactccgaacaccggttggaaaactgataaatatggtaccctgtacaaatctgaatccgcttccttcacgccgaacaccgacatcattactcgcacgactggtcctttccgttccatgccgcagtccggcgtactgaaagcaggccagactatccactacgatgaagttatgaaacaggacggtcacgtttgggttggttataccggcaactccggccagcgtatttatctgccggtgcgtacttggaacaaagcgaccaacactctgggtgtactgtggggcactattaaa

SEQ ID NO: 121 - DNA, 735 bp, synthetic construct
  source 1..735: mol_type = other DNA
gctactcatgagcactccgcacagtggctgaataactacaagaaaggctacggctacggcccatacccgctgaccattaacggctctatgcactacggtgtggacttctttatgaacatcggtactccggtaaaagcaatctcctctggcaaaattgtcgaggcaggctggagcaactacggtggcggtaaccagattggcctgattgaaaacgatggcgttcaccgtcaatggtacatgcacctgtccaagtacaacgtcaaagtaggtgattacgttaaagcgggccagattatcggttggagcggctctaccggttattccactgcgccgcatctgcactttcagcgtatggttaactccttctccaactctaacgcgcaggacccgatgccgttcctgaaatctgccggctacggtaaagcaagcggtactgtgaccccgacccctaatactggttggaagaccaacaaatacggtactctgtataaaagcgagtctgcctccttcaccccgaacaccgatattatcacccgtacgaccggtccgtttcgtagcatgccacagtctggcgtactgaaagctggtcaaaccatccattacgatgaagtgatgaaacaggatggtcacgtttgggtgggttataccggcaactccggccaacgtatctatctgccggtacgcacctggaacaaagcgaccaacaccctgggtgtgctgtggggcaccatcaaa

SEQ ID NO: 122 - DNA, 735 bp, synthetic construct
  source 1..735: mol_type = other DNA
gcaacccatgaacatagcgcccagtggctgaataactacaagaaaggttatggctatggtccatacccgctgactatcaacggttctatgcactatggtgttgactttttcatgaatatcggcaccccagtcaaggctatcagctctggtaaaattgttgaggcaagctggtccaactacggcggtggcaaccagatcggcctgatcgaaaacgatggcgttcaccgtcagtggtacatgcacctgagcaagtacaacgttaaagtcggcgactatgttaaagcaggtcagattatcggttggtccggcagcaccggttactctacggcgccgcacctgcacttccagcgtatggttaactctttctctaacagcaacgcacaggacccgatgccgttcctgaaatctgcgggttacggcaaagcatccggcaccgttaccccgaccccaaacactggctggaaaactaacaagtacggtactctgtacaaatctgaaagcgcaagcttcaccccgaacacggatattatcactcgtactaccggtccgttccgctccatgccgcagagcggtgttctgaaagcgggtcagactatccactacgatgaggttatgaaacaggatggccatgtgtgggtaggttacaccggtaactctggccagcgtatctacctgccggttcgtacttggaacaaagcaaccaataccctcggcgtactgtggggcaccatcaag

SEQ ID NO: 123 - DNA, 735 bp, synthetic construct
  source 1..735: mol_type = other DNA
gcgactcacgaacactccgcccagtggctgaataactacaagaaaggttacggctacggtccttacccgctgaccatcaatggcagcatgcattacggcgtggattttttcatgaacatcggcacgccagtgaaagcaatctcctctggcaaaatcgttgaagcttcttggtctaactacggttccggcaaccagatcggtctgatcgaaaacgatggcgtgcaccgccaatggtatatgcacctcagcaaatacaacgttaaagtaggcgactatgtcaaagccggtcaaattatcggttggagcggttctacgggttacagcactgcaccgcacctgcacttccagcgtatggtgaactctttctccaacagcaacgctcaggacccgatgccgttcctgaaatccgcaggttatggtaaagctagcggtacggtgaccccgacgccgaacaccggttggaaaaccaacaaatacggcaccctctacaaatccgaatccgcgtccttcaccccgaacaccgatattatcacccgtactaccggtccgttccgttctatgccgcagtctggcgtgctgaaagcgggccaaaccattcattatgatgaagtgatgaaacaggatggtcacgtgtgggtgggttatactggtaactccggtcagcgcatctacctgccggtgcgtacctggaacaaagcgaccaacaccctgggcgtcctgtggggtaccattaaa

SEQ ID NO: 124 - DNA, 735 bp, synthetic construct
  source 1..735: mol_type = other DNA
gccactcacgagcactctgctcagtggctgaataactacaagaaaggttatggctacggtccgtacccactgacgattcagggttctatgcattacggcgtggactttttcatgaacatcggcacgccggtaaaggcgatcagctctggcaagatcgtagaggccggttggtctaactacggcggtggcaaccagatcggcctcattgaaaacgacggtgtgcaccgtcaatggtatatgcacctgtccaaatataacgttaaggtgggtgattatgtgaaagctggccagatcattggttggtctggctccaccggctattctactgcgccgcatctgcactttcagcgtatggttaactctttcagcaactctaatgctcaggatccaatgccatttctgaaatccgcgggttacggcaaagcgtctggtaccgtaaccccgacccctaacacgggctggaagaccaacaaatatggtacgctgtataaatccgagtctgcgtccttcactccgaatactgacattatcacccgtactaccggtcctttccgtagcatgccgcagtccggcgtgctgaaagctggccaaaccatccattacgatgaagttatgaaacaggatggtcatgtttgggtcggctacaccggtaacagcggtcagcgtatctacctgcctgtccgtacctggaataaagccaccaacactctgggcgtactgtggggtactatcaaa

SEQ ID NO: 125 - DNA, 735 bp, synthetic construct
  source 1..735: mol_type = other DNA
gctacccacgaacacagcgcacagtggctgaataactacaaaaagggttacggttatggtccatatccgctgacgattcatggttccatgcattatggcgttgacttctttatgaacatcggtaccccggtgaaggcgatctctagcggtaaaatcgtagaagctggttggagcaactatggcggtggcaatcaaatcggtctgatcgaaaatgacggtgtacaccgccagtggtatatgcacctgtccaaatacaatgtgaaagtcggcgattacgttaaggcgggtcagattatcggttggagcggctctactggctactccaccgcaccgcacctgcactttcaacgtatggtaaatagcttttctaactctaacgctcaggacccgatgccgttcctgaaatctgccggttacggcaaagcatctggcacggttactccgactccgaacactggttggaagaccaacaagtacggtacgctgtacaagagcgaaagcgccagcttcaccccgaacactgacattatcacccgcactaccggtccgtttcgtagcatgccgcaatctggcgtgctgaaagcgggccaaaccattcactacgacgaggtgatgaaacaggacggtcatgtctgggtaggttacaccggcaactctggccagcgtatctacctcccggtccgcacttggaacaaagcaaccaacaccctgggtgtcctgtggggcactattaag

SEQ ID NO: 126 - DNA, 426 bp, synthetic construct
  source 1..426: mol_type = other DNA
gcaactcatgagcactctgcgcagtggctgaataactataagaaaggttacggttacggcccgtacccgctgactattaacggcagcatgcactatggcgttgatttctttatgaacatcggtaccccggttaaagcgatttcctctggcaaaattgttgaagcgtcttggtccaattatggtggcggtaaccagatcggcctgatcgaaaacgacggtgtacatcgccagtggtacatgcacctgagcaaatacaacgtaaaagttggtgactacgttaaagcaggtcagattatcggctggtctggttccaccggctactctacggcgcctcacctgcactttcagcgtatggttaatagcttctctaactctaacgcccaggacccgatgccgttcctgaagtccgccggctatggtaaagcg

SEQ ID NO: 127 - DNA, 426 bp, synthetic construct
  source 1..426: mol_type = other DNA
gcaacccacgaacactccgctcagtggctgaataactacaagaaaggctacggttacggcccttatccgctgaccatcaacggctccatgcactatggtgtggactttttcatgaacatcggcacgccggtgaaagcgatcagctccggtaaaattgtagaagccagctggagcaactacggctctggcaaccaaatcggcctgattgaaaacgatggcgttcaccgtcagtggtatatgcacctgtctaagtacaacgtcaaggtgggtgactatgttaaagcaggtcagattatcggttggtccggcagcactggctattccactgcaccacacctgcattttcagcgcatggtgaactctttcagcaactccaacgcacaggacccaatgccattcctgaagtccgctggttatggcaaggca